(19) United States
(12) Patent Application Publication
(10) Pub. No.: US 2023/0363911 A1
(43) Pub. Date: Nov. 16, 2023

(54) HEART VALVE SEALING DEVICES AND DELIVERY DEVICES THEREFOR

(71) Applicant: EDWARDS LIFESCIENCES CORPORATION, Irvine, CA (US)

(72) Inventors: Florian Georg Deuschl, Laguna Beach, CA (US); Chris J. Okos, Huntington Beach, CA (US); Waina Michelle Chu, Tustin, CA (US); Jian Lin Phan, Fountain Valley, CA (US); Lauren R. Freschauf, Mission Viejo, CA (US); Wen Yan Chen, Garden Grove, CA (US)

(21) Appl. No.: 18/352,860

(22) Filed: Jul. 14, 2023

Related U.S. Application Data
(63) Continuation of application No. PCT/US2022/012266, filed on Jan. 13, 2022.
(60) Provisional application No. 63/138,309, filed on Jan. 15, 2021.

(51) Int. Cl. A61F 2/24

(52) U.S. Cl. CPC A61F 2/2463; A61F 2/2436; A61F 2220/0075

(57) ABSTRACT

An implantable device is configured to be positioned within a native heart valve to allow the native heart valve to form a more effective seal. The implantable device includes a cover. The cover can be attached to one or more sleeves, can have a portion that has a lower coefficient of friction, and/or can have separate pieces that cover different portions of the implantable device.

### CROSS-REFERENCE TO RELATED APPLICATIONS

This application is a continuation of Patent Cooperation Treaty Application No. PCT/US2022/012266, filed on Jan. 13, 2022, which claims the benefit of U.S. Provisional Application No. 63/138,309, filed on Jan. 15, 2021, the contents of which are incorporated by reference in their entireties.

### BACKGROUND OF THE INVENTION

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant prosthetic devices in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a prosthetic device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

An implantable device or implant (e.g., implantable prosthetic device, etc.) is configured to be positioned within a native heart valve to allow the native heart valve to form a more effective seal. The device includes a first cover portion and a second cover portion. The second cover portion has a lower coefficient of friction than the first cover portion.

In some implementations, the implantable device or implant has at least one anchor. The at least one anchor is configured to attach the device to at least one leaflet of a native heart valve. The device includes a first cover portion and a second cover portion. The second cover portion has a lower coefficient of friction than the first cover portion.

In some implementations, an implantable device or implant includes a plurality of paddles, a first cover portion, and a second cover portion. The first and second cover portions are attached to the plurality paddles. The second cover portion has a lower coefficient of friction than the first cover portion.

In some implementations, an implantable device or implant includes a coaptation portion, an anchor portion, and first and second cover portions. The anchor portion comprises a plurality of paddles moveably connected to the coaptation portion. The first and second cover portions cover one or more of the coaptation portion and the anchor portion. The second cover portion has a lower coefficient of friction than the first cover portion.

An example implantable device or implant has a coaptation element and at least one anchor. The coaptation element is configured to be positioned within the native heart valve orifice to help fill a space where the native valve is regurgitant and form a more effective seal. The coaptation element can have a structure that is impervious to blood and that allows the native leaflets to close around the coaptation element during ventricular systole to block blood from flowing from the left or right ventricle back into the left or right atrium, respectively. The coaptation element can be connected to leaflets of the native valve by the anchor. The implantable device or implant also includes first and second cover portions. The second cover portion has a lower coefficient of friction than the first cover portion.

In some implementations, an implantable device or implant includes an anchor portion and one or more sleeves. The anchor portion is configured to attach to one or more leaflets of a native heart valve and includes one or more anchors. Each anchor has a paddle frame. The one or more sleeves are attached to the paddle frame, and each sleeve is lubricious to facilitate movement of the device through native structures of a patient's heart.

In some implementations, an implantable device or implant includes an anchor portion, one or more sleeves, and a cover. The anchor portion is configured to attach to one or more leaflets of a native heart valve and includes one or more anchors. Each anchor has a paddle frame. The one or more sleeves are attached to the paddle frame, and the cover is attached to the one or more sleeves and covers at least a portion of the paddle frame.

An example implantable device or implant includes a coaptation portion, an anchor portion, and a cover assembly. The coaptation portion has a coaptation element. The anchor portion is configured to attach to one or more leaflets of a native heart valve and includes first and second anchors. Each of the first and second anchors has a paddle frame, an inner paddle, and outer paddle, and a clasp. The cover assembly includes a first cover for covering at least a portion of the paddle frame of both of the first and second anchors, a pair of second covers in which one second cover covers at least a portion of the inner paddle of the first anchor and the other second cover covers at least a portion of the inner paddle of the second anchor, and a third cover for covering at least a portion of the coaptation element and the clasps of the first and second anchors.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. It is appreciated that these drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 is another cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a front view of the implantable device or implant of FIG. 22;

FIG. 24 shows a side view of the implantable device or implant of FIG. 22;

FIG. 25 shows a front view of the implantable device or implant of FIG. 22 with a cover covering the paddles and a coaptation element or spacer;

FIG. 26 shows a top perspective view of the implantable device or implant of FIG. 22 in an open position;

FIG. 27 shows a bottom perspective view of the implantable device or implant of FIG. 22 in an open position;

FIG. 28 shows a clasp for use in an implantable device or implant;

FIG. 29 shows a portion of native valve tissue grasped by a clasp;

FIG. 30 shows a side view of an example implantable device or implant in a partially-open position with clasps in a closed position;

FIG. 31 shows a side view of an example implantable device or implant in a partially-open position with clasps in an open position;

FIG. 32 shows a side view of an example implantable device or implant in a half-open position with clasps in a closed position;

FIG. 33 shows a side view of an example implantable device or implant in a half-open position with clasps in an open position;

FIG. 34 shows a side view of an example implantable device or implant in a three-quarters-open position with clasps in a closed position;

FIG. 35 shows a side view of an example implantable device or implant in a three-quarters-open position with clasps in an open position;

FIG. 36 shows a side view of an example implantable device in a fully open or full bailout position with clasps in a closed position;

FIG. 37 shows a side view of an example implantable device in a fully open or full bailout position with clasps in an open position;

FIGS. 38-49 show the example implantable device or implant of FIGS. 30-38, including a cover, being delivered and implanted within a native valve;

FIG. 50 is a schematic view illustrating a path of native valve leaflets along each side of a coaptation element or spacer of an example valve repair device or implant;

FIG. 51 is a top schematic view illustrating a path of native valve leaflets around a coaptation element or spacer of an example valve repair device or implant;

FIG. 52 illustrates a coaptation element or spacer in a gap of a native valve as viewed from an atrial side of the native valve;

FIG. 53 illustrates a valve repair device or implant attached to native valve leaflets with the coaptation element or spacer in the gap of the native valve as viewed from a ventricular side of the native valve;

FIG. 54 is a perspective view of a valve repair device or implant attached to native valve leaflets with the coaptation element or spacer in the gap of the native valve shown from a ventricular side of the native valve;

FIG. 55 shows a perspective view of an example implantable device or implant in a closed position;

FIG. 56 shows a perspective view of an example clasp of an example implantable device or implant in a closed position;

FIG. 57 shows a front view of an example implantable device or implant in a closed condition including a cover shown in broken lines;

FIG. 58 shows a front view of the example implantable device or implant of FIG. 57 with the cover illustrated in solid lines;

FIG. 59 shows a side view of the example implantable device or implant of FIG. 58;

FIG. 60 shows a top view of the example implantable device or implant of FIG. 58;

FIG. 61 shows a bottom view of the example implantable device or implant of FIG. 58;

FIG. 62 shows a front view of the example implantable device or implant of FIG. 58 in an open condition;

FIG. 63 shows a side view of the example implantable device or implant of FIG. 62;

FIG. 64 shows a top view of the example implantable device or implant of FIG. 62;

FIG. 65 shows a bottom view of the example implantable device or implant of FIG. 62;

FIG. 66 shows a top view of an example implantable device or implant in an open condition;

FIG. 67 shows a bottom view of the example implantable device or implant of FIG. 66;

FIG. 68 shows a top view of an example implantable device or implant in an open condition;

FIG. 69 shows a bottom view of the example implantable device or implant of FIG. 68;

FIG. 70 shows a top view of an example implantable device or implant in an open condition;

FIG. 71 shows a bottom view of the example implantable device or implant of FIG. 70;

FIG. 72 shows a front view of an example implantable device or implant in a closed condition including a cover shown in broken lines;

FIG. 73 shows a front view of the example implantable device or implant of FIG. 72 with the cover illustrated in solid lines;

FIG. 74 shows a side view of the example implantable device or implant of FIG. 73;

FIG. 75 shows a top view of the example implantable device or implant of FIG. 73;

FIG. 76 shows a bottom view of the example implantable device or implant of FIG. 73;

FIG. 77 shows a front view of the example implantable device or implant of FIG. 73 in an open condition;

FIG. 78 shows a side view of the example implantable device or implant of FIG. 77;

FIG. 79 shows a top view of the example implantable device or implant of FIG. 77;

FIG. 80 shows a bottom view of the example implantable device or implant of FIG. 77;

FIG. 81 shows a top view of an example implantable device or implant in an open condition;

FIG. 82 shows a bottom view of the example implantable device or implant of FIG. 81;

FIG. 83 shows a top view of an example implantable device or implant in an open condition;

FIG. 84 shows a bottom view of the example implantable device or implant of FIG. 83;

FIG. 85 shows a top view of an example implantable device or implant in an open condition;

FIG. 86 shows a bottom view of the example implantable device or implant of FIG. 85;

FIG. 87 shows a side view of an example cover for an implantable device or implant;

FIG. 88 is a cross-sectional view taken along the plane indicated by lines 88-88 in FIG. 87;

FIG. 89 is a cross-sectional view taken along the plane indicated by lines 89-89 in FIG. 87;

FIG. 90 shows a side view of an example cover for an implantable device or implant;

FIG. 91 is a cross-sectional view taken along the plane indicated by lines 91-91 in FIG. 90;

FIG. 92 is a cross-sectional view taken along the plane indicated by lines 91-91 in FIG. 90 with the cover inside out;

FIG. 93 shows a first side of a first knitted material for covering an example implantable device or implant;

FIG. 94 shows a second side of the first knitted material of FIG. 93;

FIG. 95 shows a first side of a second knitted material for covering an example implantable device or implant;

FIG. 96 shows a second side of the second knitted material of FIG. 95;

FIG. 97 shows a chart comparing the forces experienced by a probe covered with the coverings shown in FIGS. 93-96 with the first sides of the knitted materials arranged on the exterior;

FIG. 98 shows a chart comparing the forces experienced by a probe covered with the coverings shown in FIGS. 93-96 with the second sides of the knitted materials arranged on the exterior;

FIG. 99 shows a first side of a first woven material for covering an example implantable device or implant, of which a second side would be similar in appearance;

FIG. 100 shows a first side of a second woven material for covering an example implantable device or implant, of which a second side would be similar in appearance;

FIG. 101 shows a chart comparing the forces experienced by a probe covered with the coverings shown in FIGS. 99-100 with the first sides of the woven materials arranged on the exterior;

FIG. 102 shows a chart comparing the forces experienced by a probe covered with the coverings shown in FIGS. 99-100 with the second sides of the woven materials arranged on the exterior;

FIG. 103 shows a perspective view of an example implantable device having paddles with adjustable widths;

FIG. 104 is a cross-section of the implantable device of FIG. 103 in which the implantable device is bisected;

FIG. 105 is another cross-section of the implantable device of FIG. 103 in which the implantable device is bisected along a plane perpendicular to the plane shown in FIG. 104;

FIG. 106 is a schematic illustration of an example implant catheter assembly coupled to the implantable device of FIG. 103, in which an actuation element, such as a tube is coupled to a paddle actuation control and to a driver head of the implantable device;

FIG. 107 is an illustration of the assembly of FIG. 106 with the implantable device rotated 90 degrees to show the paddle width adjustment element coupled to a movable member of the implantable device and coupled to a paddle width control;

FIG. 108 shows a perspective view of an example sleeve for attaching to paddle frames of an implantable device;

FIG. 109 shows a perspective view of an example implantable device that includes a plurality of the example sleeves of FIG. 108 and an example covering;

FIG. 110 shows another perspective view of the example implantable device of FIG. 109;

FIG. 111 shows a front view of the example implantable device of FIG. 109;

FIG. 112 shows a side view of the example implantable device of FIG. 109;

FIG. 113 shows an example inner paddle cover of the example covering of FIG. 109;

FIG. 114 shows an example coaptation element cover of the example covering of FIG. 109;

FIG. 115 shows an example paddle frame cover of the example covering of FIG. 109;

FIG. 116 shows the example implantable device of FIG. 109, where the covering includes a clasp cover;

FIG. 117 shows a partial view of a clasp and the clasp cover of FIG. 116;

FIG. 117A shows an example clasp cover for covering the clasp of FIG. 109;

FIG. 118 shows an example connection between a tether and paddle frames of a pair of paddles for the implantable device of FIG. 109; and

FIG. 119 shows a schematic view of the example connection between the tether and paddle frames of FIG. 116 shown in area A of FIG. 118.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of implantable devices, valve repair devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques and methods herein can be performed on a living animal or on a simulation, such as on a cadaver, cadaver heart, simulator (e.g., with the body parts, heart, tissue, etc. being simulated), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

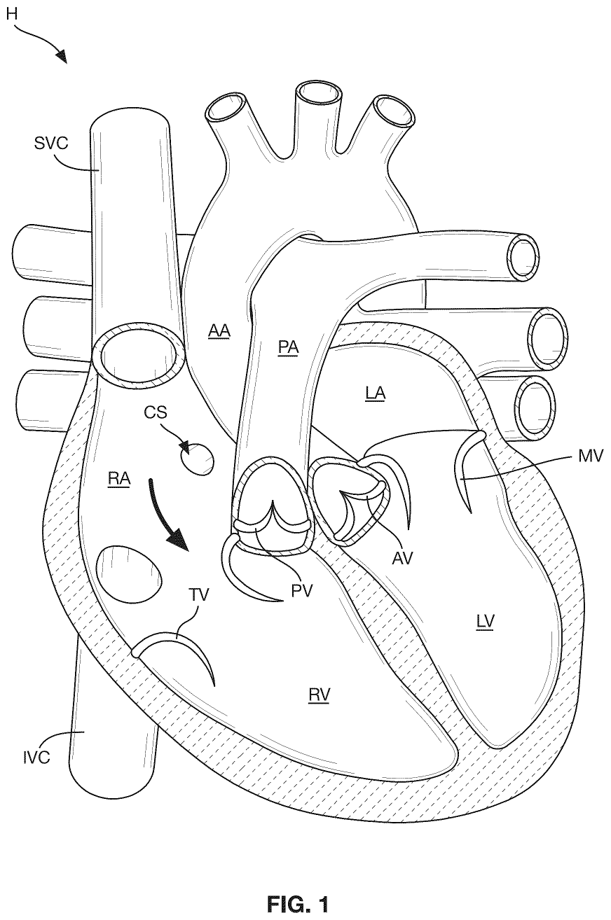

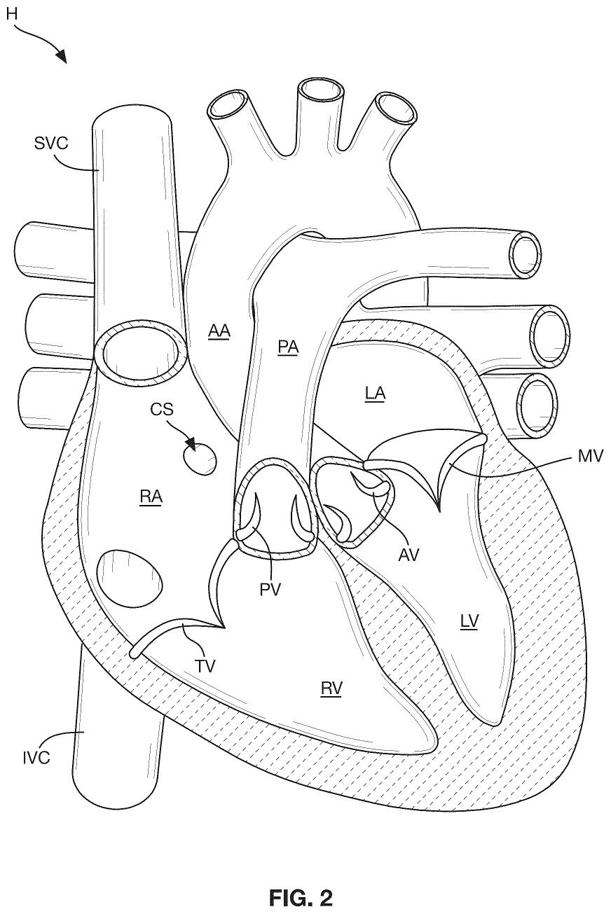

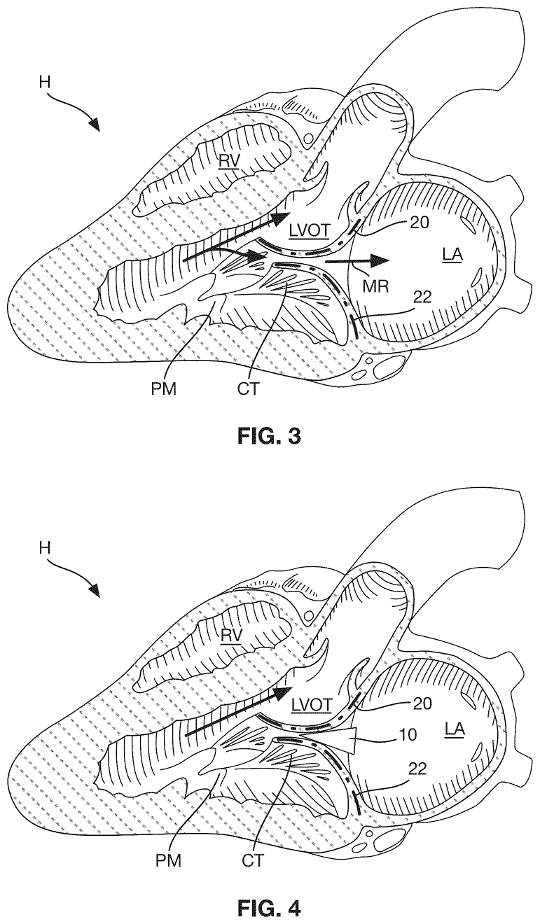

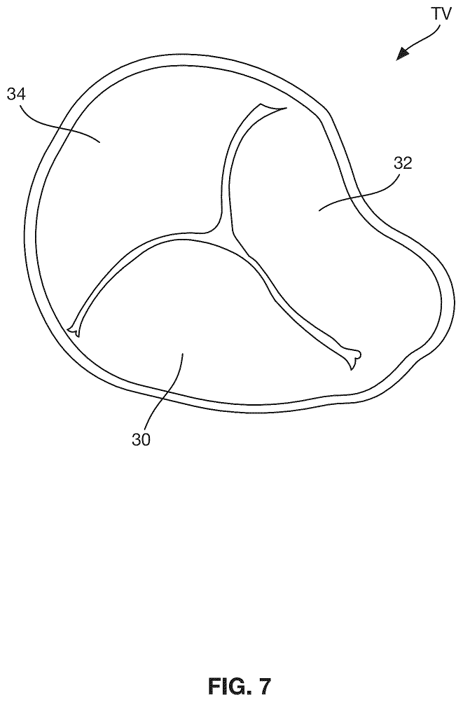

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) can distort a native valve's geometry, which can cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant or incompetent-which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

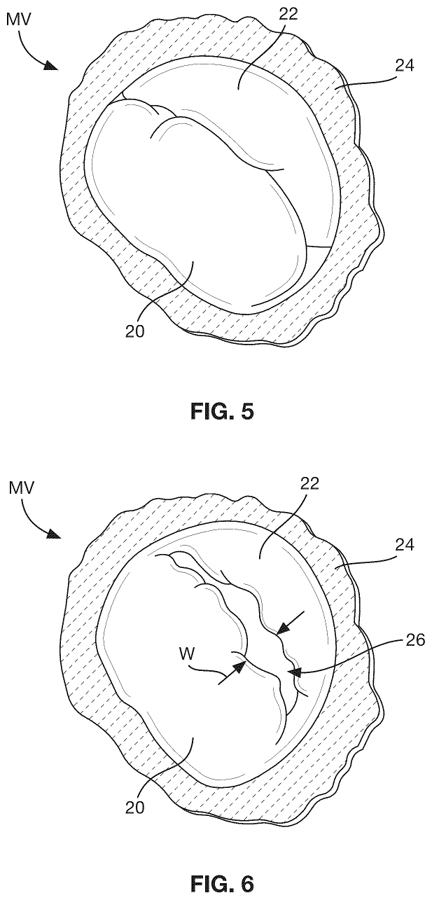

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent regurgitation of blood through the mitral valve MV. As can be seen in FIG. 4, an abstract representation of an implantable device, valve repair device, or implant 10 is shown implanted between the leaflets 20, 22 such that regurgitation does not occur during systole (compare FIG. 3 with FIG. 4). In some implementations, the coaptation element (e.g., spacer, coaption element, coaptation member, gap filler, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).).

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves may either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, prevents the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device (e.g., implantable prosthetic device, etc.) or implant can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element (e.g., coaption element, spacer, etc.) is configured to be positioned within the native heart valve orifice to help fill the space between the leaflets and form a more effective seal, thereby reducing or preventing regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device may be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaption element, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surface that extends between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element, such as a shaft or actuation wire, to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element, e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still-compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for various implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

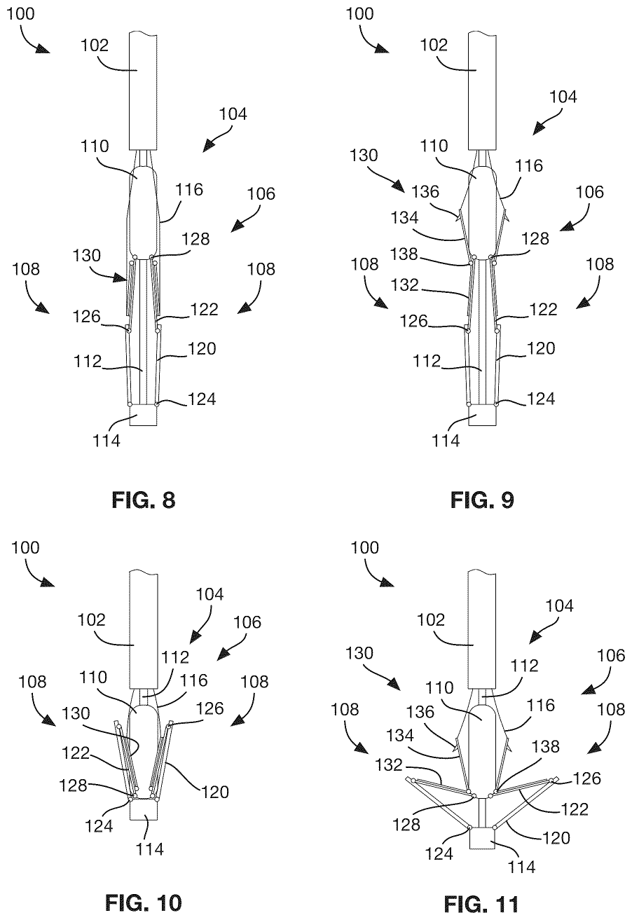

Referring now to FIGS. 8-15, a schematically illustrated implantable device or implant 100 (e.g., a prosthetic spacer device, valve repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety for all purposes. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system or other means for delivery 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 112 (e.g., actuation wire, actuation shaft, actuation tube, etc.). The anchor portion 106 includes one or more anchors 108 that are actuatable between open and closed conditions and can take a wide variety of forms, such as, for example, paddles, gripping elements, or the like. Actuation of the means for actuating or actuation element 112 opens and closes the anchor portion 106 of the device 100 to grasp the native valve leaflets during implantation. The means for actuating or actuation element 112 (as well as other means for actuating and actuation elements herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and the means for coapting or coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and means for actuating or actuation element 112 (e.g., actuation wire, actuation shaft, etc.). These can be configured to extend through a guide catheter/sheath (e.g., a transseptal sheath, etc.). In some implementations, the means for actuating or actuation element 112 extends through a delivery catheter and the means for coapting or coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element removably attaches the coaptation element 110 to the delivery system 102, either directly or indirectly, so that the means for actuating or actuation element 112 slides through the collar or other attachment element and, in some implementations, through a means for coapting or coaptation element 110 during actuation to open and close the paddles 120, 122 of the anchor portion 106 and/or anchors 108.

In some implementation, the anchor portion 106 and/or anchors 108 can include attachment portions or gripping members. The illustrated gripping members can comprise clasps 130 that include a base or fixed arm 132, a moveable arm 134, optional barbs, friction-enhancing elements, or other means for securing 136 (e.g., protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate means for coapting or coaptation element 110. In some implementations, the clasps (e.g., barbed clasps, etc.) have flat surfaces and do not fit in a recess of the inner paddle. Rather, the flat portions of the clasps are disposed against the surface of the inner paddle 122. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the barbs, friction-enhancing elements, or means for securing 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. This pinching force remains constant regardless of the position of the inner paddles 122. Optional barbs, friction-enhancing elements, or other means for securing 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a means for coapting or coaptation element 110. The clasps 130 can be used to grasp and/or further secure the native leaflets by engaging the leaflets with barbs, friction-enhancing elements, or means for securing 136 and pinching the leaflets between the moveable and fixed arms 134, 132. The barbs, friction-enhancing elements, or other means for securing 136 (e.g., barbs, protrusions, ridges, grooves, textured surfaces, adhesive, etc.) of the clasps or barbed clasps 130 increase friction with the leaflets or may partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of a catheter 102 in the fully open position, because the fully open position takes up the least space and allows the smallest catheter to be used (or the largest device 100 to be used for a given catheter size). In the elongated condition the cap 114 is spaced apart from the means for coapting or coaptation element 110 such that the paddles 120, 122 are fully extended. In some implementations, an angle formed between the interior of the outer and inner paddles 120, 122 is approximately 180 degrees. The clasps 130 are kept in a closed condition during deployment through the delivery system 102 so that the barbs, friction-enhancing elements, or other means for securing 136 (FIG. 9) do not catch or damage the delivery system 102 or tissue in the patient's heart. The actuation lines 116 can extend through the coupler 117, around the collar 115, and attach to the moveable arms 134.

Referring now to FIG. 9, the device 100 is shown in an elongated detangling condition, similar to FIG. 8, but with the clasps 130 in a fully open position, ranging from about 140 degrees to about 200 degrees, from about 170 degrees to about 190 degrees, or about 180 degrees between fixed and moveable portions 132, 134 of the clasps 130. Fully opening the paddles 120, 122 and the clasps 130 has been found to improve ease of detanglement or detachment from anatomy of the patient, such as the chordae tendineae CT, during implantation of the device 100.

Referring now to FIG. 10, the device 100 is shown in a shortened or fully closed condition. The compact size of the device 100 in the shortened condition allows for easier maneuvering and placement within the heart. To move the device 100 from the elongated condition to the shortened condition, the means for actuating or actuation element 112 is retracted to pull the cap 114 towards the means for coapting or coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the means for coapting or coaptation element 110 cause the paddles or gripping elements to move radially outward. During movement from the open to closed position, the outer paddles 120 maintain an acute angle with the means for actuating or actuation element 112. The outer paddles 120 can optionally be biased toward a closed position. The inner paddles 122 during the same motion move through a considerably larger angle as they are oriented away from the means for coapting or coaptation element 110 in the open condition and collapse along the sides of the means for coapting or coaptation element 110 in the closed condition. In some implementations, the inner paddles 122 are thinner and/or narrower than the outer paddles 120, and the connection portions 126, 128 (e.g., joints, flexible connections, etc.) connected to the inner paddles 122 can be thinner and/or more flexible. For example, this increased flexibility can allow more movement than the connection portion 124 connecting the outer paddle 120 to the cap 114. In some implementations, the outer paddles 120 are narrower than the inner paddles 122. The connection portions 126, 128 connected to the inner paddles 122 can be more flexible, for example, to allow more movement than the connection portion 124 connecting the outer paddle 120 to the cap 114. In some implementations, the inner paddles 122 can be the same or substantially the same width as the outer paddles.

Referring now to FIGS. 11-13, the device 100 is shown in a partially open, grasp-ready condition. To transition from the fully closed to the partially open condition, the means for actuating or actuation element (e.g., actuation wire, actuation shaft, etc.) is extended to push the cap 114 away from the means for coapting or coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single means for actuating or single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. For example, the device 100 can have two actuation elements and two independent caps (or other attachment portions), such that one independent actuation element (e.g., wire, shaft, etc.) and cap (or other attachment portion) are used to control one paddle, and the other independent actuation element and cap (or other attachment portion) are used to control the other paddle.

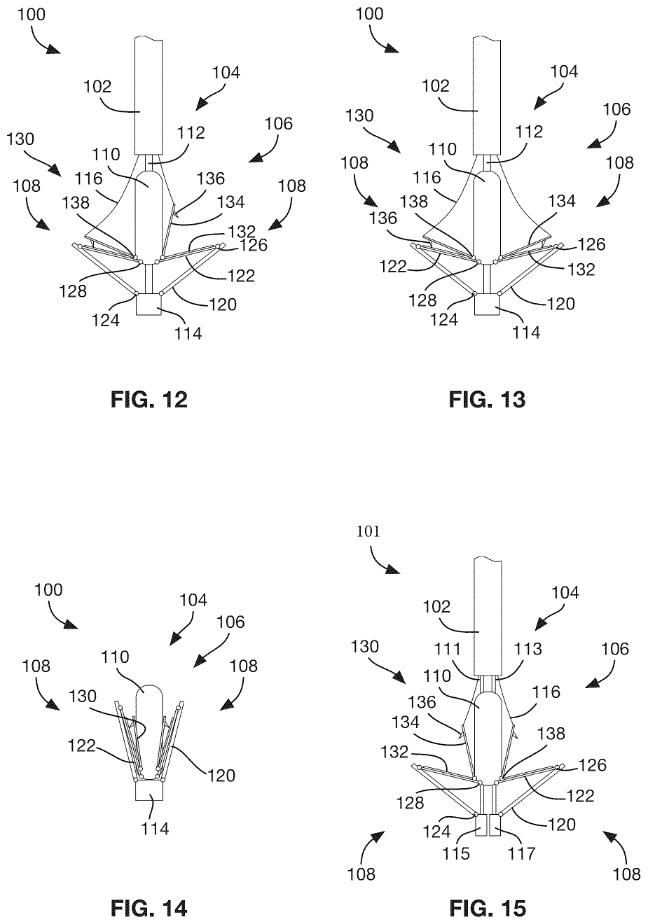

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system or means for delivery 102 and means for actuating or actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the means for coapting or coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 100 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the means for actuating or actuation element 111 is extended to push the cap 115 away from the means for coapting or coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the means for actuating or actuation element 113 is extended to push the cap 115 away from the means for coapting or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single means for actuating or actuation element 112.

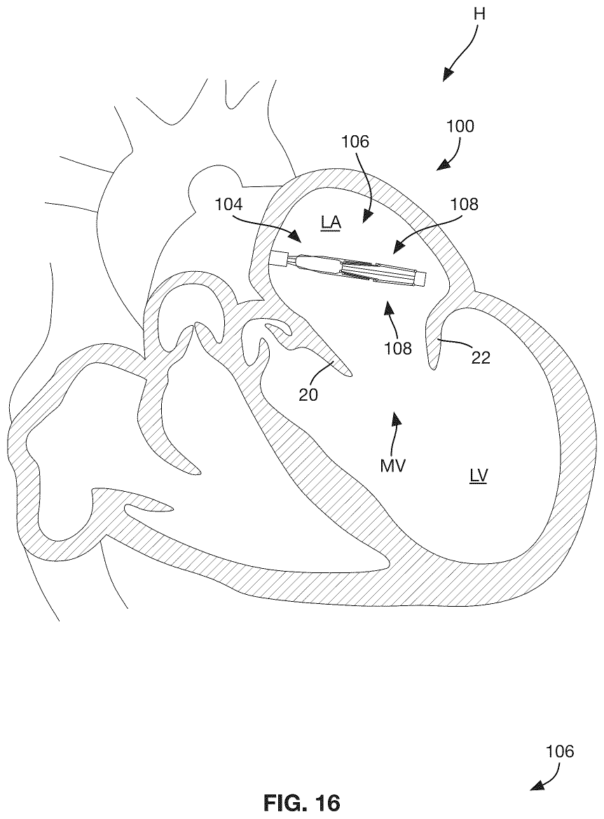

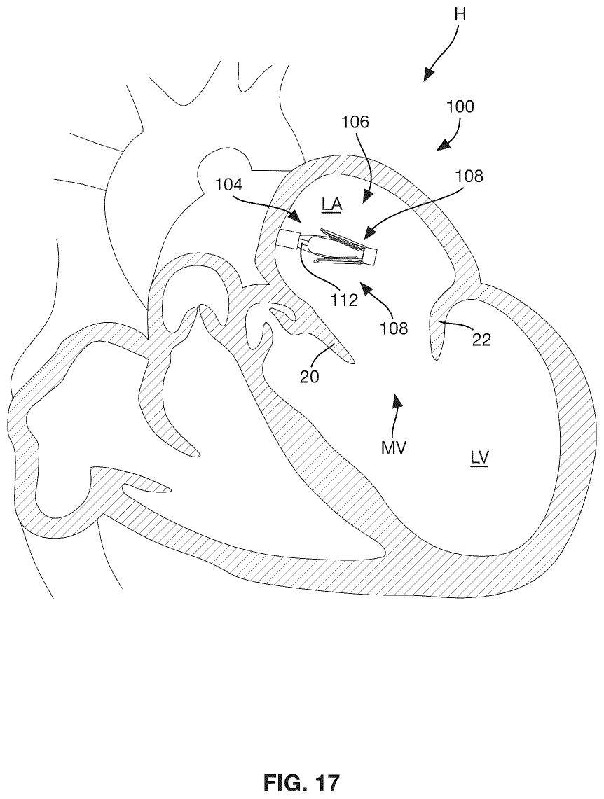

Referring now to FIGS. 16-21, the implantable device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The means for actuating or actuation element 112 is then retracted to move the implant/device into the fully closed condition shown in FIG. 17.

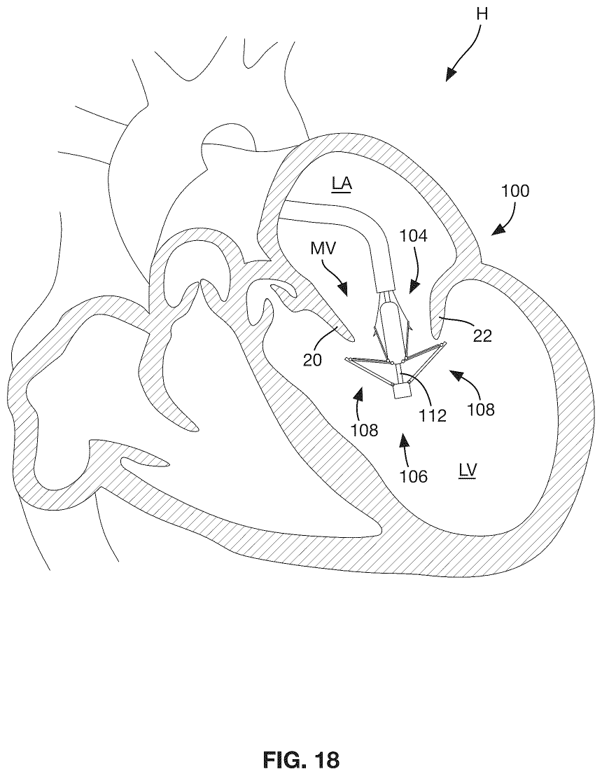

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

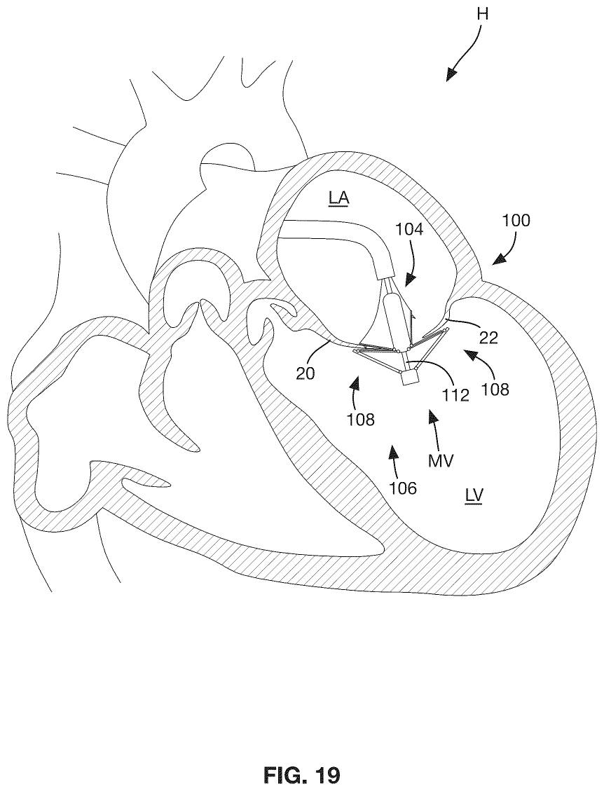

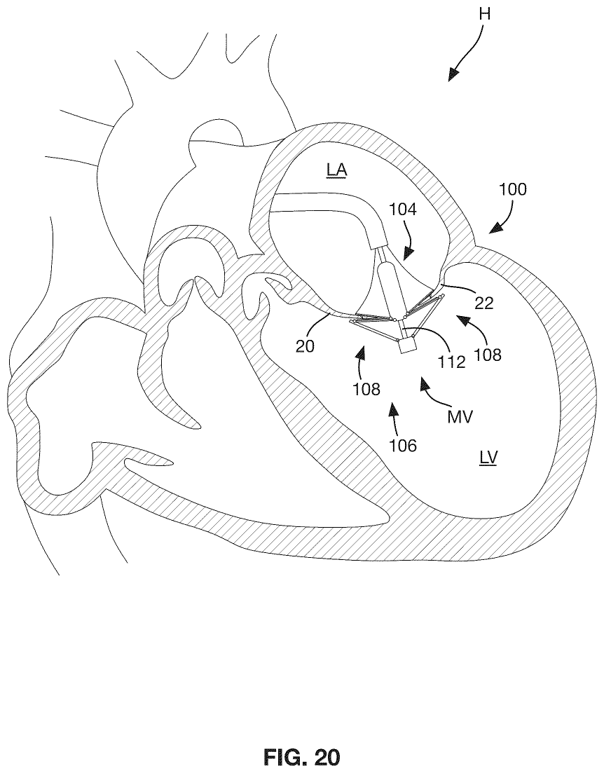

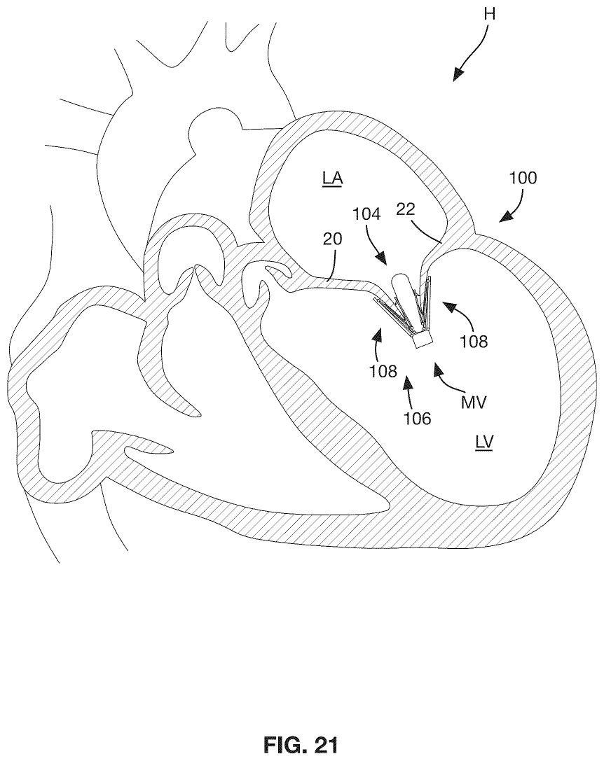

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), means for actuating or actuation element 112 and actuation lines 116 are then retracted and the device or implant 100 is fully closed and deployed in the native mitral valve MV.

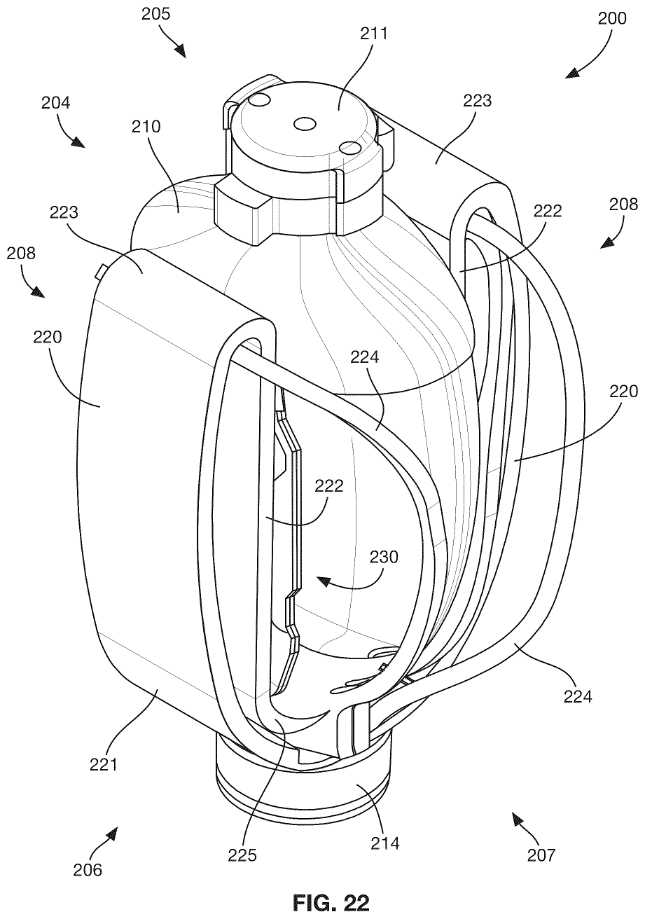

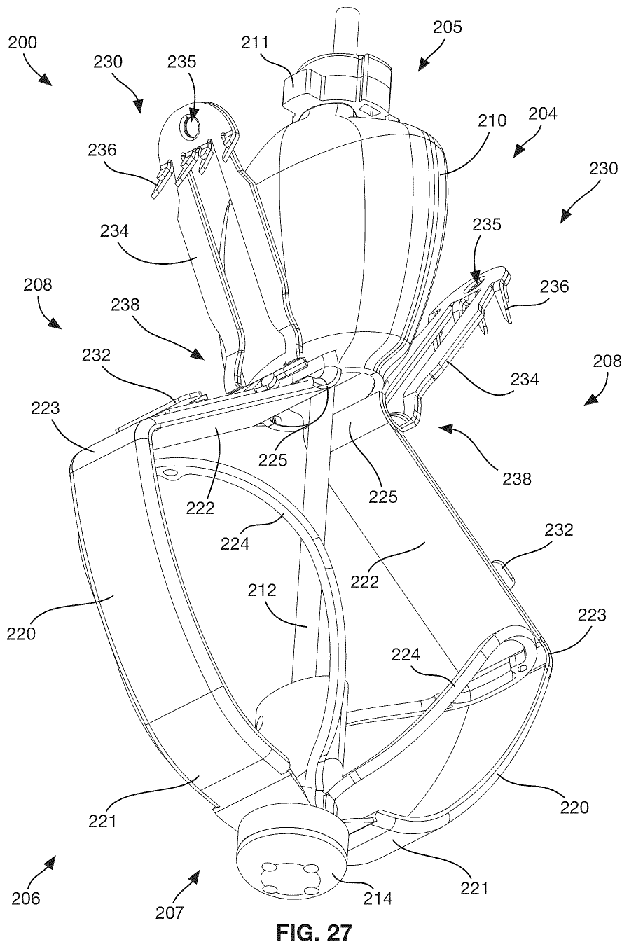

Referring now to FIGS. 22-27, an example of an implantable device or implant or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

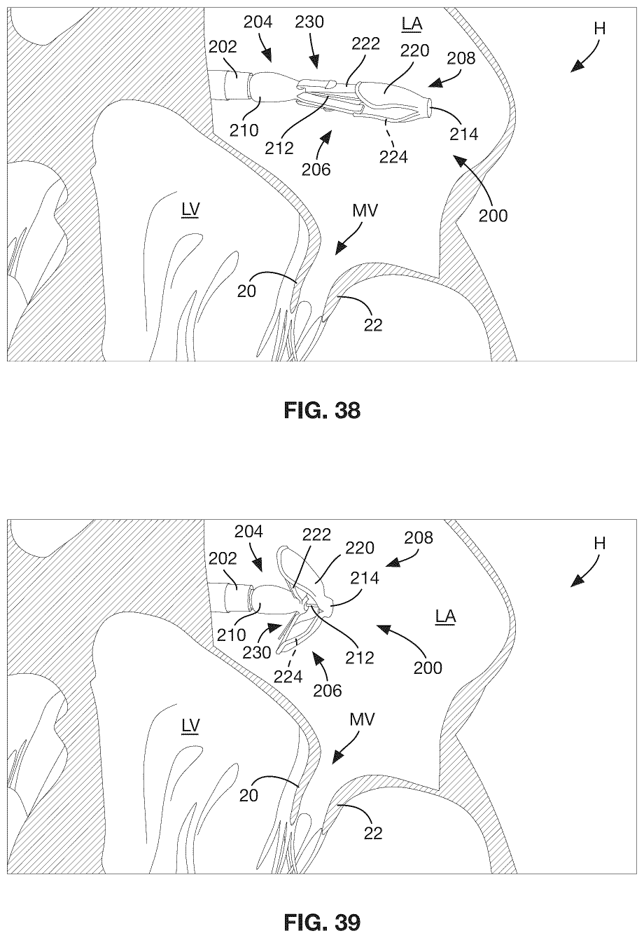

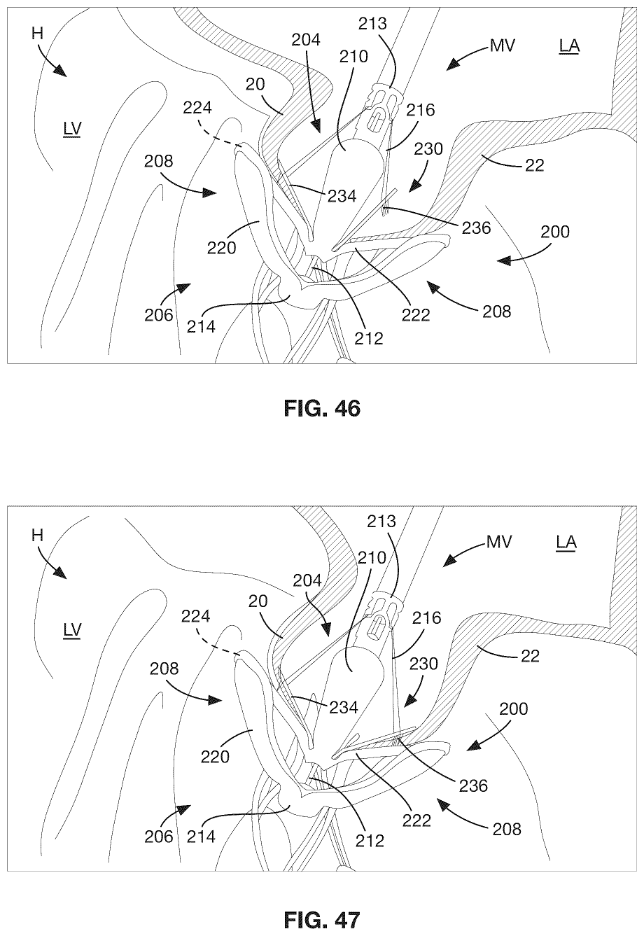

In some implementations, the implantable device or implant 200 includes a coaptation portion 204, a proximal or attachment portion 205, an anchor portion 206, and a distal portion 207. In some implementations, the coaptation portion 204 of the device optionally includes a coaptation element 210 (e.g., a spacer, coaption element, plug, membrane, sheet, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 205 includes a first or proximal collar 211 (or other attachment element) for engaging with a capture mechanism 213 (FIGS. 43-49) of a delivery system 202 (FIGS. 38-42 and 49). Delivery system 202 can be the same as or similar to delivery system 102 described elsewhere and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element 212 (e.g., actuation shaft, actuation rod, actuation tube, actuation wire, actuation line, etc.) extends from the delivery system 202 to engage and enable actuation of the implantable device or implant 200. In some implementations, the actuation element 212 extends through the capture mechanism 213, proximal collar 211, and coaptation element 210 to engage a cap 214 of the distal portion 207. The actuation element 212 can be configured to removably engage the cap 214 with a threaded connection, or the like, so that the actuation element 212 can be disengaged and removed from the device 200 after implantation.

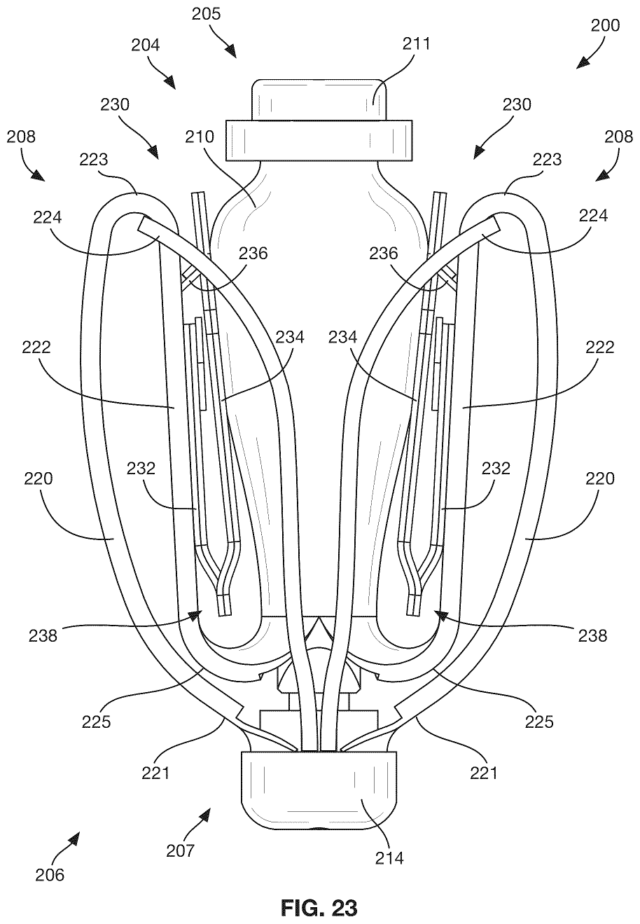

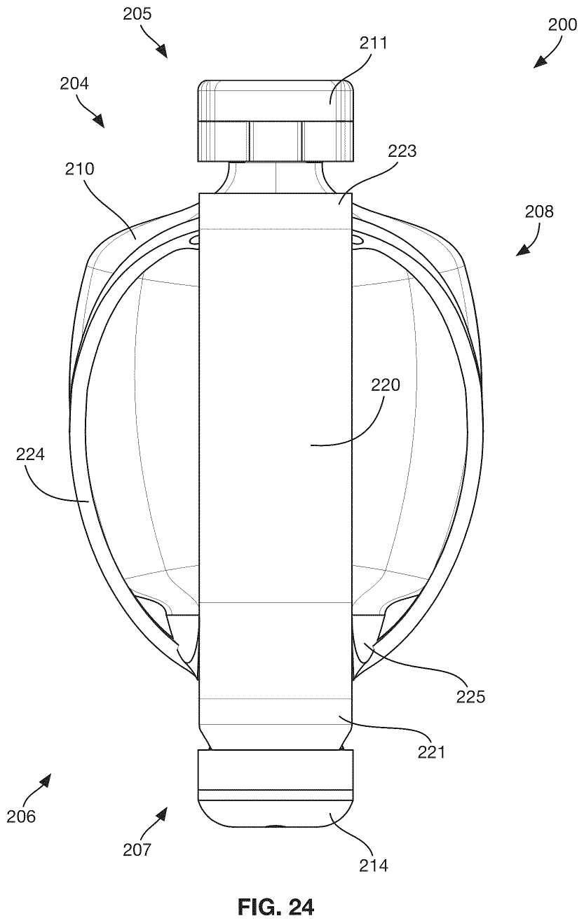

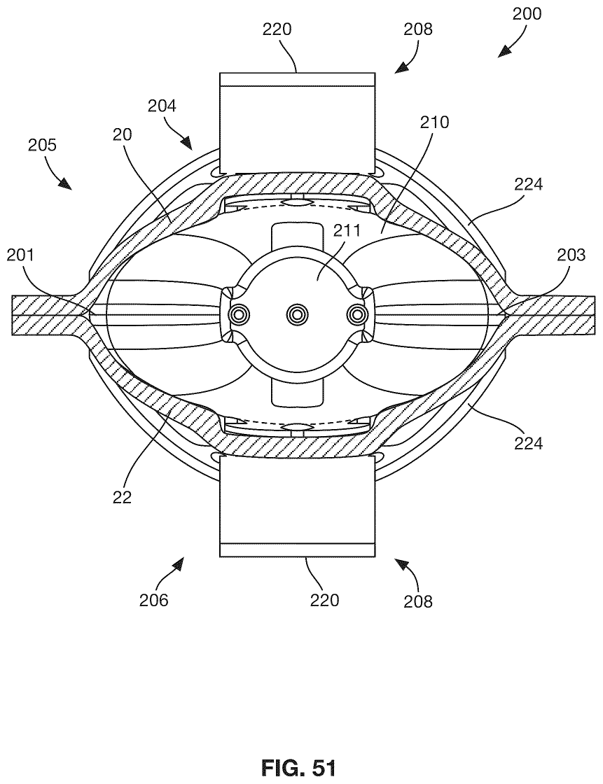

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above (e.g., FIG. 51) and has a tapered shape or cross-section when seen from a front view (e.g., FIG. 23) and a round shape or cross-section when seen from a side view (e.g., FIG. 24). A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion 232 of the clasps 230. The stiffening of the inner paddle allows the device to move to the various different positions shown and described herein. The inner paddle 222, the outer paddle 220, the coaptation can all be interconnected as described herein, such that the device 200 is constrained to the movements and positions shown and described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 provide additional pinching force between the inner paddles 222 and the coaptation element 210 and assist in wrapping the leaflets around the sides of the coaptation element 210 for a better seal between the coaptation element 210 and the leaflets, as can be seen in FIG. 51. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

Configuring the paddle frames 224 in this manner provides increased surface area compared to the outer paddles 220 alone. This can, for example, make it easier to grasp and secure the native leaflets. The increased surface area can also distribute the clamping force of the paddles 220 and paddle frames 224 against the native leaflets over a relatively larger surface of the native leaflets in order to further protect the native leaflet tissue. Referring again to FIG. 51, the increased surface area of the paddle frames 224 can also allow the native leaflets to be clamped to the implantable device or implant 200, such that the native leaflets coapt entirely around the coaptation member or coaptation element 210. This can, for example, improve sealing of the native leaflets 20, 22 and thus prevent or further reduce mitral regurgitation.

In some implementations the clasps comprise a moveable arm coupled to the anchors. In some implementations, the clasps 230 include a base or fixed arm 232, a moveable arm 234, barbs 236, and a joint portion 238. The fixed arms 232 are attached to the inner paddles 222, with the joint portion 238 disposed proximate the coaptation element 210. The joint portion 238 is spring-loaded so that the fixed and moveable arms 232, 234 are biased toward each other when the clasp 230 is in a closed condition. In some implementations, the clasps 230 include friction-enhancing elements or means for securing, such as barbs, protrusions, ridges, grooves, textured surfaces, adhesive, etc.

In some implementations, the fixed arms 232 are attached to the inner paddles 222 through holes or slots 231 with sutures (not shown). The fixed arms 232 can be attached to the inner paddles 222 with any suitable means, such as screws or other fasteners, crimped sleeves, mechanical latches or snaps, welding, adhesive, clamps, latches, or the like. The fixed arms 232 remain substantially stationary relative to the inner paddles 222 when the moveable arms 234 are opened to open the clasps 230 and expose the barbs or other friction-enhancing elements 236. The clasps 230 are opened by applying tension to actuation lines 216 (e.g., as shown in FIGS. 43-48) attached to holes 235 in the moveable arms 234, thereby causing the moveable arms 234 to articulate, pivot, and/or flex on the joint portions 238.

Referring now to FIG. 29, a close-up view of one of the leaflets 20, 22 grasped by a clasp such as clasp 230 is shown. The leaflet 20, 22 is grasped between the moveable and fixed arms 234 of the clasp 230. The tissue of the leaflet 20, 22 is not pierced by the barbs or friction-enhancing elements 236, though in some implementations the barbs 236 may partially or fully pierce through the leaflet 20, 22. The angle and height of the barbs or friction-enhancing elements 236 relative to the moveable arm 234 helps to secure the leaflet 20, 22 within the clasp 230. In particular, a force pulling the implant off of the native leaflet 20, 22 will encourage the barbs or friction-enhancing elements 236 to further engage the tissue, thereby ensuring better retention. Retention of the leaflet 20, 22 in the clasp 230 is further improved by the position of fixed arm 232 near the barbs/friction-enhancing elements 236 when the clasp 230 is closed. In this arrangement, the tissue is formed by the fixed arms 232 and the moveable arms 234 and the barbs/friction-enhancing elements 236 into an S-shaped torturous path. Thus, forces pulling the leaflet 20, 22 away from the clasp 230 will encourage the tissue to further engage the barbs/friction-enhancing elements 236 before the leaflets 20, 22 can escape. For example, leaflet tension during diastole can encourage the barbs 236 to pull toward the end portion of the leaflet 20, 22. Thus, the S-shaped path can utilize the leaflet tension during diastole to more tightly engage the leaflets 20, 22 with the barbs/friction-enhancing elements 236.

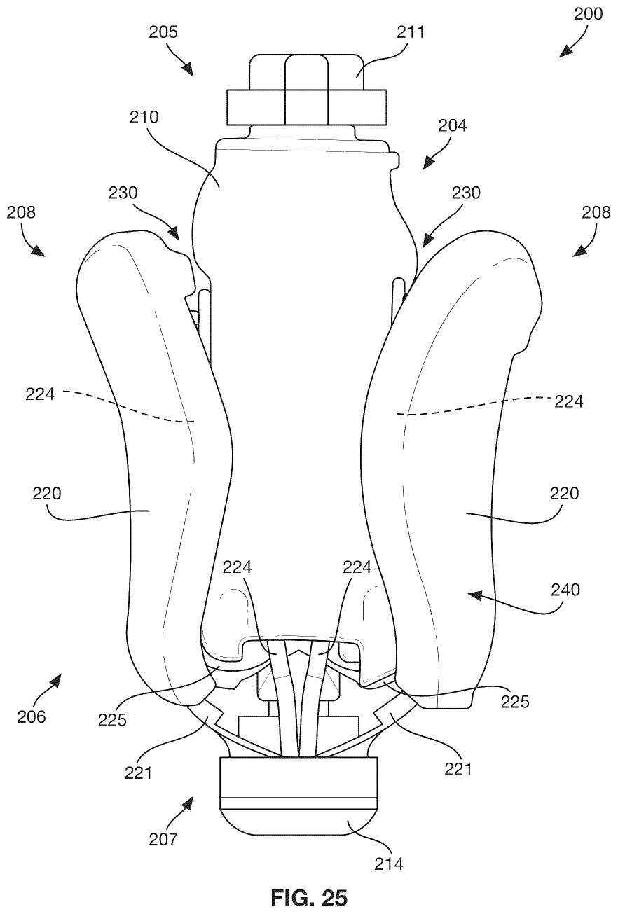

Referring to FIG. 25, the device or implant 200 can also include a cover 240. In some implementations, the cover 240 can be disposed on the coaptation element 210, the outer and inner paddles 220, 222, and/or the paddle frames 224. The cover 240 can be configured to prevent or reduce blood-flow through the device or implant 200 and/or to promote native tissue ingrowth. In some implementations, the cover 240 can be a cloth or fabric such as PET, velour, or other suitable fabric. In some implementations, in lieu of or in addition to a fabric, the cover 240 can include a coating (e.g., polymeric) that is applied to the implantable device or implant 200.

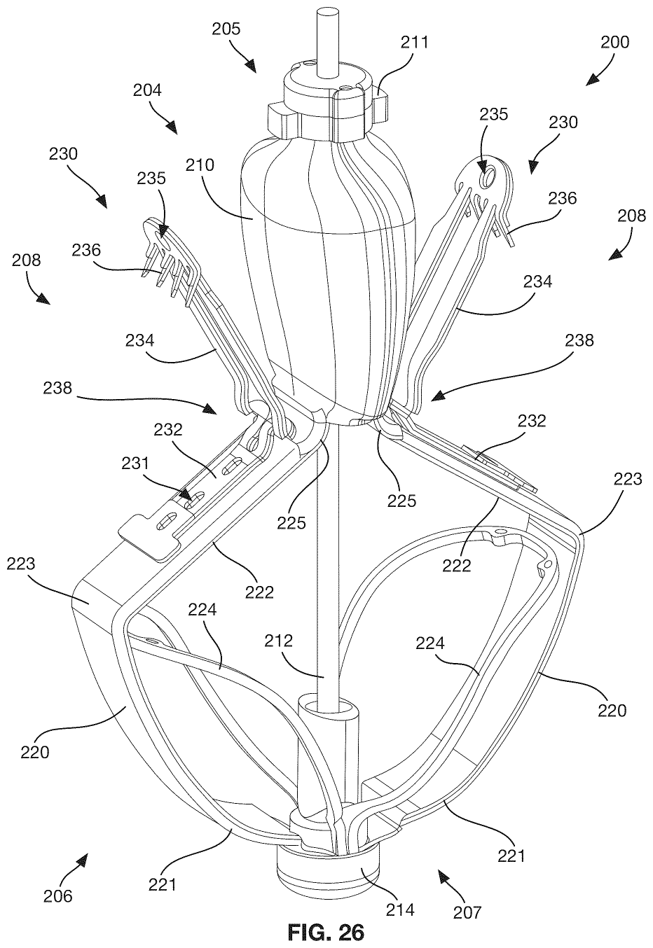

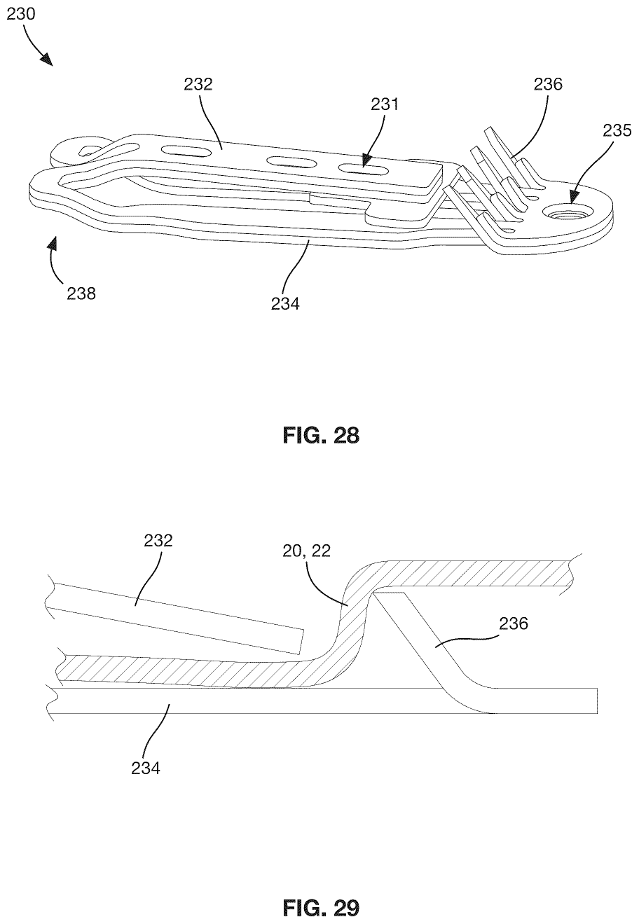

During implantation, the paddles 220, 222 of the anchors 208 are opened and closed to grasp the native valve leaflets 20, 22 between the paddles 220, 222 and the coaptation element 210. The anchors 208 are moved between a closed position (FIGS. 22-25) to various open positions (FIGS. 26-37) by extending and retracting the actuation element 212. Extending and retracting the actuation element 212 increases and decreases the spacing between the coaptation element 210 and the cap 214, respectively. The proximal collar 211 (or other attachment element) and the coaptation element 210 slide along the actuation element 212 during actuation so that changing of the spacing between the coaptation element 210 and the cap 214 causes the paddles 220, 220 to move between different positions to grasp the mitral valve leaflets 20, 22 during implantation.

As the device 200 is opened and closed, the pair of inner and outer paddles 222, 220 are moved in unison, rather than independently, by a single actuation element 212. Also, the positions of the clasps 230 are dependent on the positions of the paddles 222, 220. For example, the clasps 230 are arranged such that closure of the anchors 208 simultaneously closes the clasps 230. In some implementations, the device 200 can be made to have the paddles 220, 222 be independently controllable in the same manner (e.g., the device 100 illustrated in FIG. 15).

In some implementations, the clasps 230 further secure the native leaflets 20, 22 by engaging the leaflets 20, 22 with barbs and/or other friction-enhancing elements 236 and pinching the leaflets 20, 22 between the moveable and fixed arms 234, 232. In some implementations, the clasps 230 are barbed clasps that include barbs that increase friction with and/or may partially or completely puncture the leaflets 20, 22. The actuation lines 216 (FIGS. 43-48) can be actuated separately so that each clasp 230 can be opened and closed separately. Separate operation allows one leaflet 20, 22 to be grasped at a time, or for the repositioning of a clasp 230 on a leaflet 20, 22 that was insufficiently grasped, without altering a successful grasp on the other leaflet 20, 22. The clasps 230 can be fully opened and closed when the inner paddle 222 is not closed, thereby allowing leaflets 20, 22 to be grasped in a variety of positions as the particular situation requires.

Referring now to FIGS. 22-25, the device 200 is shown in a closed position. When closed, the inner paddles 222 are disposed between the outer paddles 220 and the coaptation element 210. The clasps 230 are disposed between the inner paddles 222 and the coaptation element 210. Upon successful capture of native leaflets 20, 22 the device 200 is moved to and retained in the closed position so that the leaflets 20, 22 are secured within the device 200 by the clasps 230 and are pressed against the coaptation element 210 by the paddles 220, 222. The outer paddles 220 can have a wide curved shape that fits around the curved shape of the coaptation element 210 to more securely grip the leaflets 20, 22 when the device 200 is closed (e.g., as can be seen in FIG. 51). The curved shape and rounded edges of the outer paddle 220 also prohibits or inhibits tearing of the leaflet tissue.

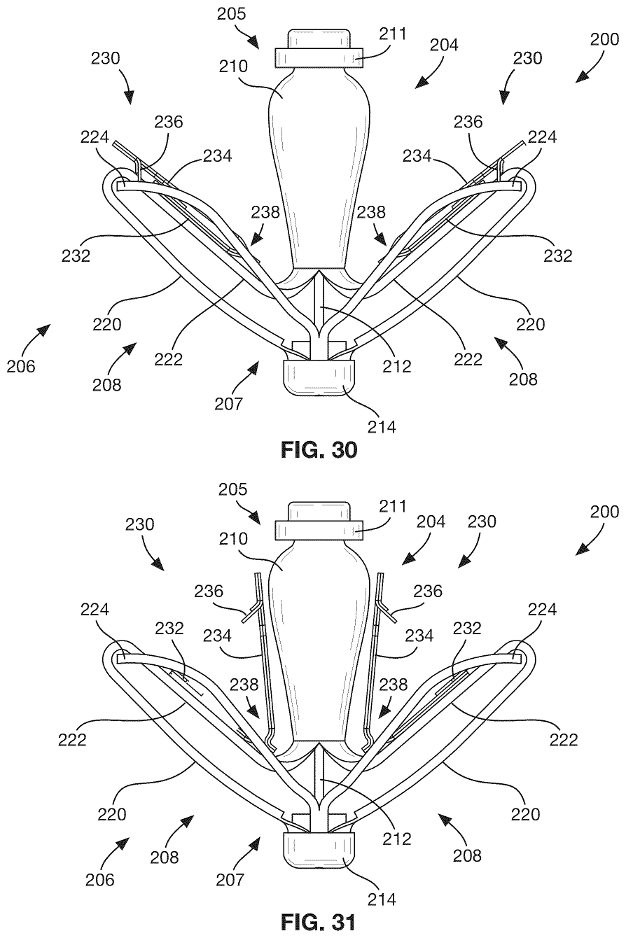

Referring now to FIGS. 30-37, the implantable device or implant 200 described above is shown in various positions and configurations ranging from partially open to fully open. The paddles 220, 222 of the device 200 transition between each of the positions shown in FIGS. 30-37 from the closed position shown in FIGS. 22-25 up extension of the actuation element 212 from a fully retracted to fully extended position.

Referring now to FIGS. 30-31, the device 200 is shown in a partially open position. The device 200 is moved into the partially open position by extending the actuation element 212. Extending the actuation element 212 pulls down on the bottom portions of the outer paddles 220 and paddle frames 224. The outer paddles 220 and paddle frames 224 pull down on the inner paddles 222, where the inner paddles 222 are connected to the outer paddles 220 and the paddle frames 224. Because the proximal collar 211 (or other attachment element) and coaptation element 210 are held in place by the capture mechanism 213, the inner paddles 222 are caused to articulate, pivot, and/or flex in an opening direction. The inner paddles 222, the outer paddles 220, and the paddle frames all flex to the position shown in FIGS. 30-31. Opening the paddles 222, 220 and frames 224 forms a gap between the coaptation element 210 and the inner paddle 222 that can receive and grasp the native leaflets 20, 22. This movement also exposes the clasps 230 that can be moved between closed (FIG. 30) and open (FIG. 31) positions to form a second gap for grasping the native leaflets 20, 22. The extent of the gap between the fixed and moveable arms 232, 234 of the clasp 230 is limited to the extent that the inner paddle 222 has spread away from the coaptation element 210.

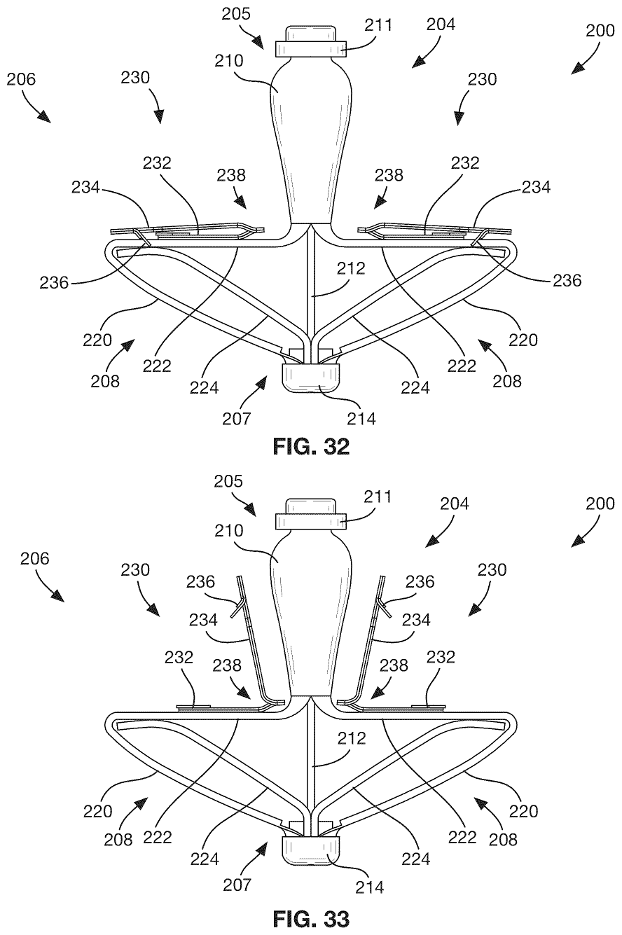

Referring now to FIGS. 32-33, the device 200 is shown in a laterally extended or open position. The device 200 is moved into the laterally extended or open position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the laterally extended or open position, the inner paddles 222 extend horizontally more than in other positions of the device 200 and form an approximately 90-degree angle with the coaptation element 210. Similarly, the paddle frames 224 are at their maximum spread position when the device 200 is in the laterally extended or open position. The increased gap between the coaptation element 210 and inner paddle 222 formed in the laterally extended or open position allows clasps 230 to open further (FIG. 33) before engaging the coaptation element 210, thereby increasing the size of the gap between the fixed and moveable arms 232, 234.

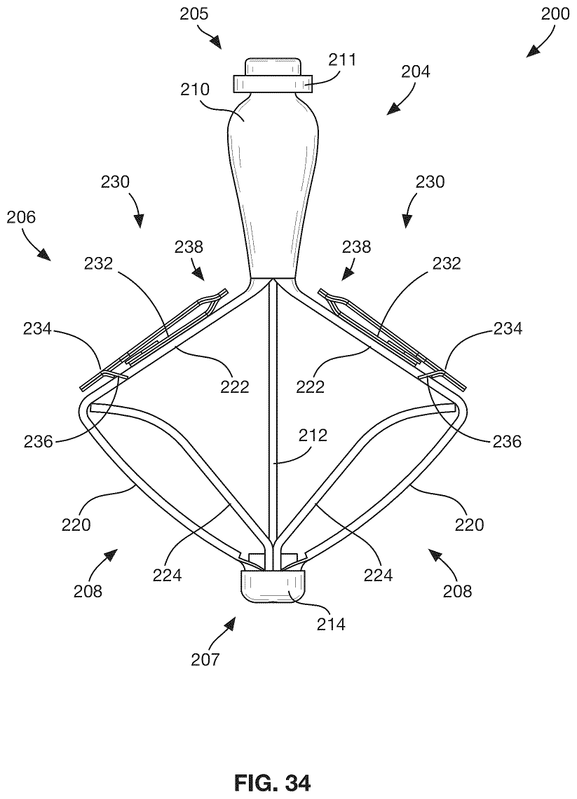

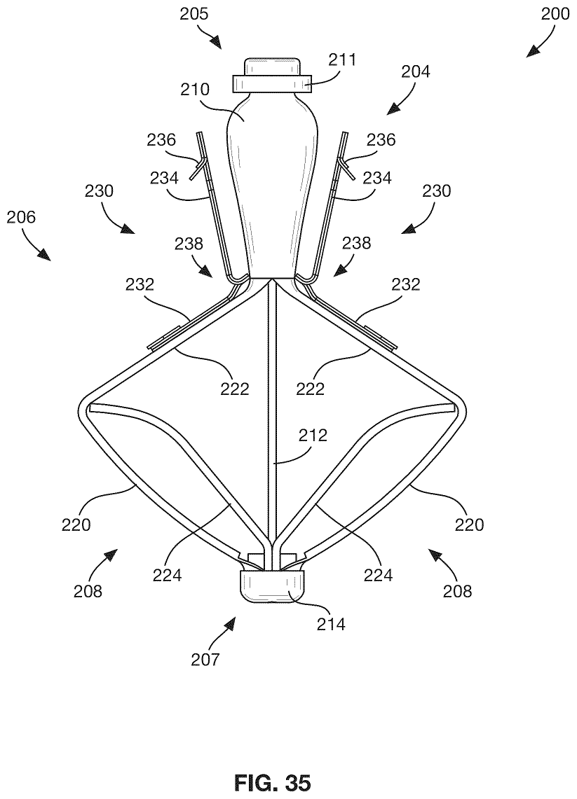

Referring now to FIGS. 34-35, the example device 200 is shown in a three-quarters extended position. The device 200 is moved into the three-quarters extended position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the three-quarters extended position, the inner paddles 222 are open beyond 90 degrees to an approximately 135-degree angle with the coaptation element 210. The paddle frames 224 are less spread than in the laterally extended or open position and begin to move inward toward the actuation element 212 as the actuation element 212 extends further. The outer paddles 220 also flex back toward the actuation element 212. As with the laterally extended or open position, the increased gap between the coaptation element 210 and inner paddle 222 formed in the laterally extended or open position allows clasps 230 to open even further (FIG. 35), thereby increasing the size of the gap between the fixed and moveable arms 232, 234.

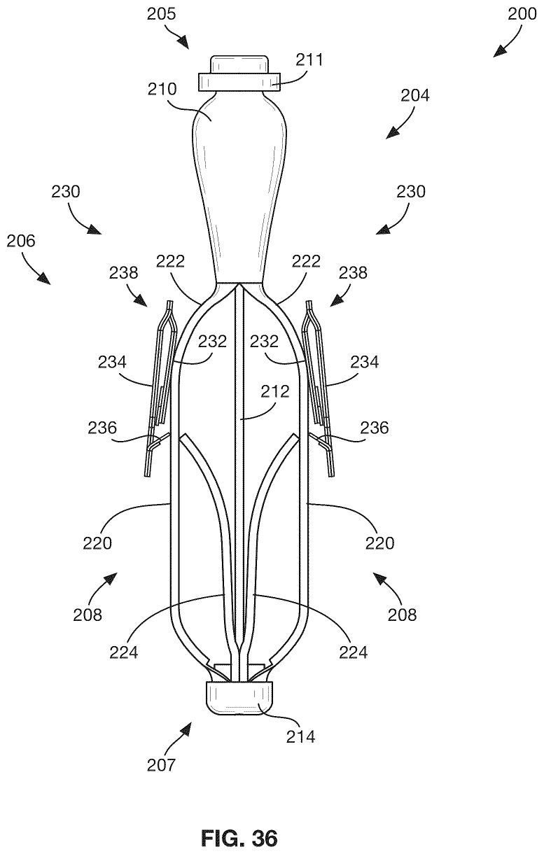

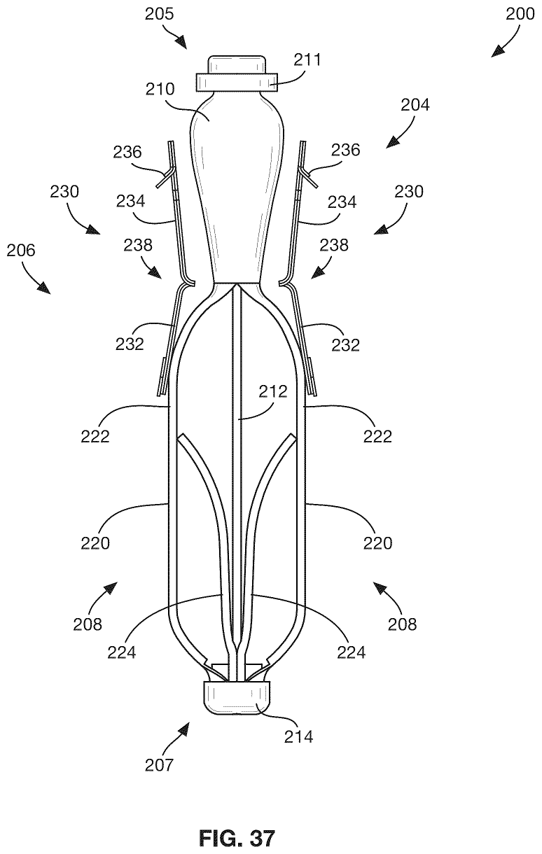

Referring now to FIGS. 36-37, the example device 200 is shown in a fully extended position. The device 200 is moved into the fully extended position by continuing to extend the actuation element 212 described above, thereby increasing the distance between the coaptation element 210 and the cap 214 of the distal portion 207 to a maximum distance allowable by the device 200. Continuing to extend the actuation element 212 pulls down on the outer paddles 220 and paddle frames 224, thereby causing the inner paddles 222 to spread apart further from the coaptation element 210. The outer paddles 220 and paddle frames 224 move to a position where they are close to the actuation element. In the fully extended position, the inner paddles 222 are open to an approximately 180-degree angle with the coaptation element 210. The inner and outer paddles 222, 220 are stretched straight in the fully extended position to form an approximately 180-degree angle between the paddles 222, 220. The fully extended position of the device 200 provides the maximum size of the gap between the coaptation element 210 and inner paddle 222, and, in some implementations, allows clasps 230 to also open fully to approximately 180 degrees (FIG. 37) between the fixed and moveable arms 232, 234 of the clasp 230. The position of the device 200 is the longest and the narrowest configuration. Thus, the fully extended position of the device 200 can be a desirable position for bailout of the device 200 from an attempted implantation or can be a desired position for placement of the device in a delivery catheter, or the like.

Configuring the device or implant 200 such that the anchors 208 can extend to a straight or approximately straight configuration (e.g., approximately 120-180 degrees relative to the coaptation element 210) can provide several advantages. For example, this configuration can reduce the radial crimp profile of the device or implant 200. It can also make it easier to grasp the native leaflets 20, 22 by providing a larger opening between the coaptation element 210 and the inner paddles 222 in which to grasp the native leaflets 20, 22. Additionally, the relatively narrow, straight configuration can prevent or reduce the likelihood that the device or implant 200 will become entangled in native anatomy (e.g., chordae tendineae CT shown in FIGS. 3 and 4) when positioning and/or retrieving the device or implant 200 into the delivery system 202.

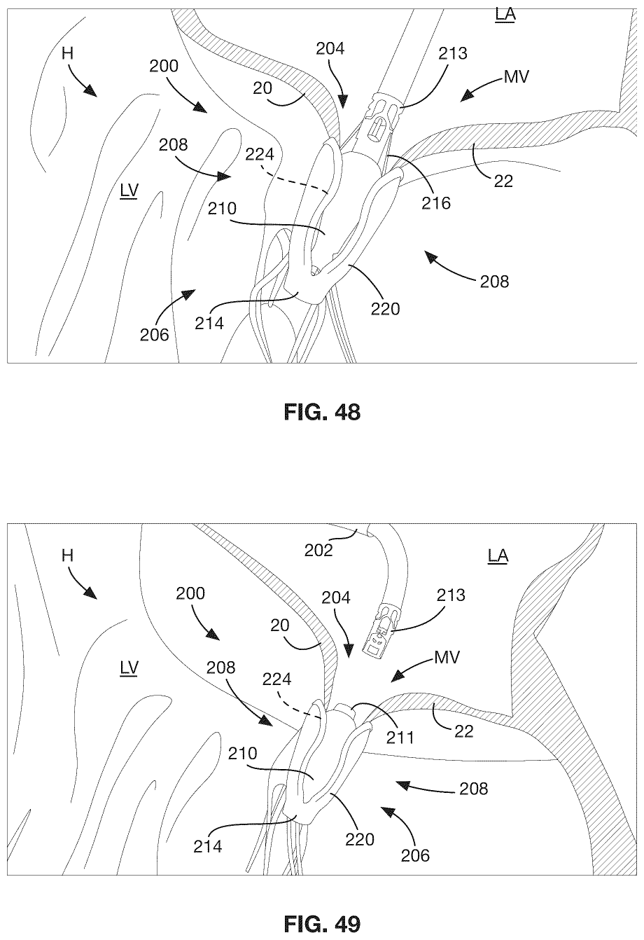

Referring now to FIGS. 38-49, an example implantable device 200 is shown being delivered and implanted within the native mitral valve MV of the heart H. As described above, the device 200 shown in FIGS. 38-49 includes the optional covering 240 (e.g., FIG. 25) over the coaptation element 210, clasps 230, inner paddles 222 and/or the outer paddles 220. The device 200 is deployed from a delivery system 202 (e.g., which can comprise an implant catheter that is extendable from a steerable catheter and/or a guide sheath) and is retained by a capture mechanism 213 (see e.g., FIGS. 43 and 48) and is actuated by extending or retracting the actuation element 212. Fingers of the capture mechanism 213 removably attach the collar 211 to the delivery system 202. In some implementations, the capture mechanism 213 is held closed around the collar 211 by the actuation element 212, such that removal of the actuation element 212 allows the fingers of the capture mechanism 213 to open and release the collar 211 to decouple the capture mechanism 213 from the device 200 after the device 200 has been successfully implanted.

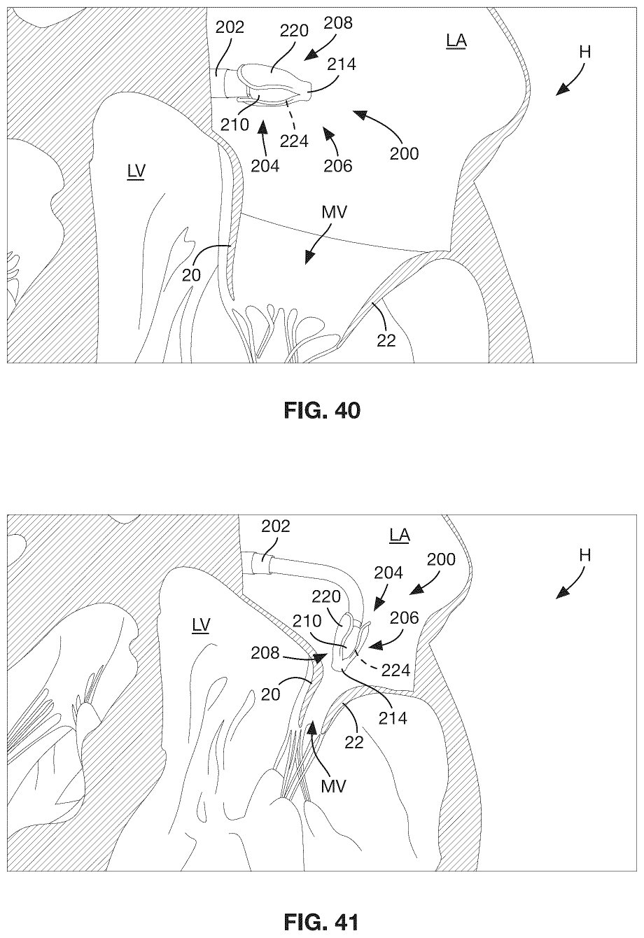

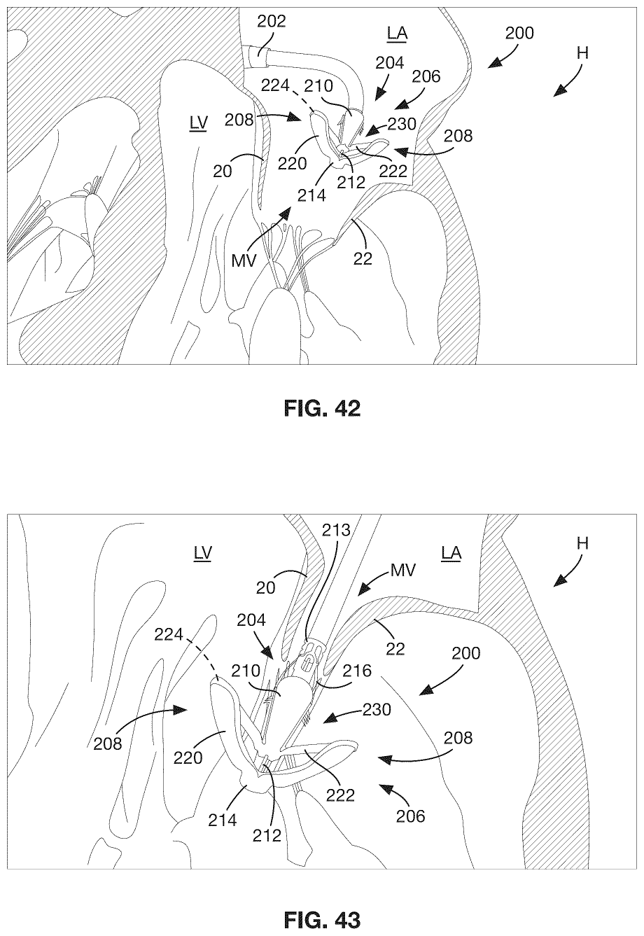

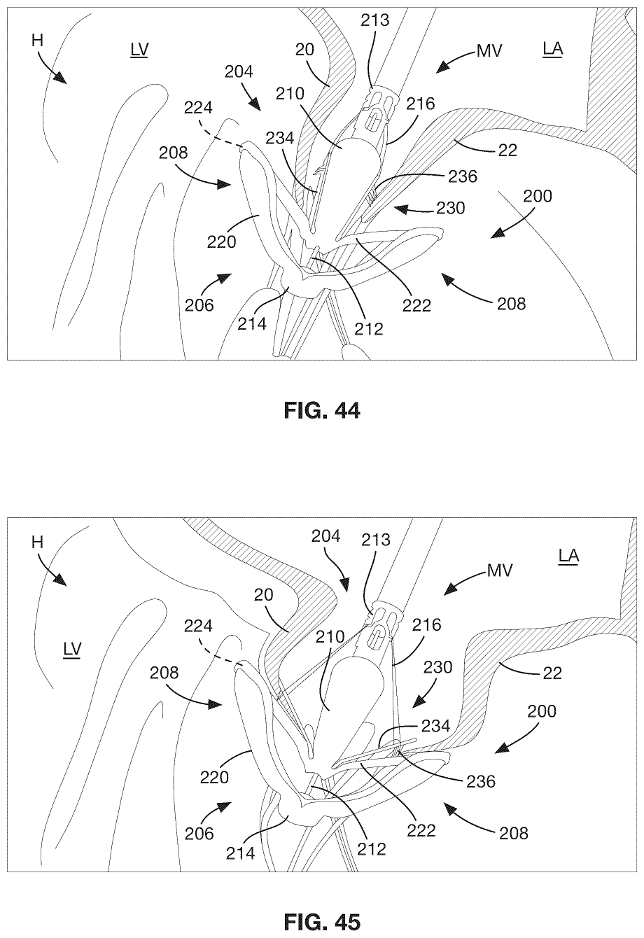

Referring now to FIG. 38, the delivery system 202 (e.g., a delivery catheter/sheath thereof) is inserted into the left atrium LA through the septum and the device/implant 200 is deployed from the delivery system 202 (e.g., an implant catheter retaining the device/implant can be extended to deploy the device/implant out from a steerable catheter) in the fully open condition for the reasons discussed above with respect to the device 100. The actuation element 212 is then retracted to move the device 200 through the partially closed condition (FIG. 39) and to the fully closed condition shown in FIGS. 40-41. Then the delivery system or catheter maneuvers the device/implant 200 towards the mitral valve MV as shown in FIG. 41. Referring now to FIG. 42, when the device 200 is aligned with the mitral valve MV, the actuation element 212 is extended to open the paddles 220, 222 into the partially opened position and the actuation lines 216 (FIGS. 43-48) are retracted to open the clasps 230 to prepare for leaflet grasp. Next, as shown in FIGS. 43-44, the partially open device 200 is inserted through the native valve (e.g., by advancing an implant catheter from a steerable catheter) until leaflets 20, 22 are properly positioned in between the inner paddles 222 and the coaptation element 210 and inside the open clasps 230.

FIG. 45 shows the device 200 with both clasps 230 closed, though the barbs 236 of one clasp 230 missed one leaflet 22. As can be seen in FIGS. 45-47, the out of position clasp 230 is opened and closed again to properly grasp the missed leaflet 22. When both leaflets 20, 22 are grasped properly, the actuation element 212 is retracted to move the device 200 into the fully closed position shown in FIG. 48. With the device 200 fully closed and implanted in the native valve, the actuation element 212 is disengaged from the cap 214 and is withdrawn to release the capture mechanism 213 from the proximal collar 211 (or other attachment element) so that the capture mechanism 213 can be withdrawn into the delivery system 202 (e.g., into a catheter/sheath), as shown in FIG. 49. Once deployed, the device 200 can be maintained in the fully closed position with a mechanical means such as a latch or can be biased to remain closed through the use of spring material, such as steel, and/or shape-memory alloys such as Nitinol. For example, the paddles 220, 222 can be formed of steel or Nitinol shape-memory alloy—produced in a wire, sheet, tubing, or laser sintered powder—and are biased to hold the outer paddles 220 closed around the inner paddles 222, coaptation element 210, and/or the clasps 230 pinched around native leaflets 20, 22.

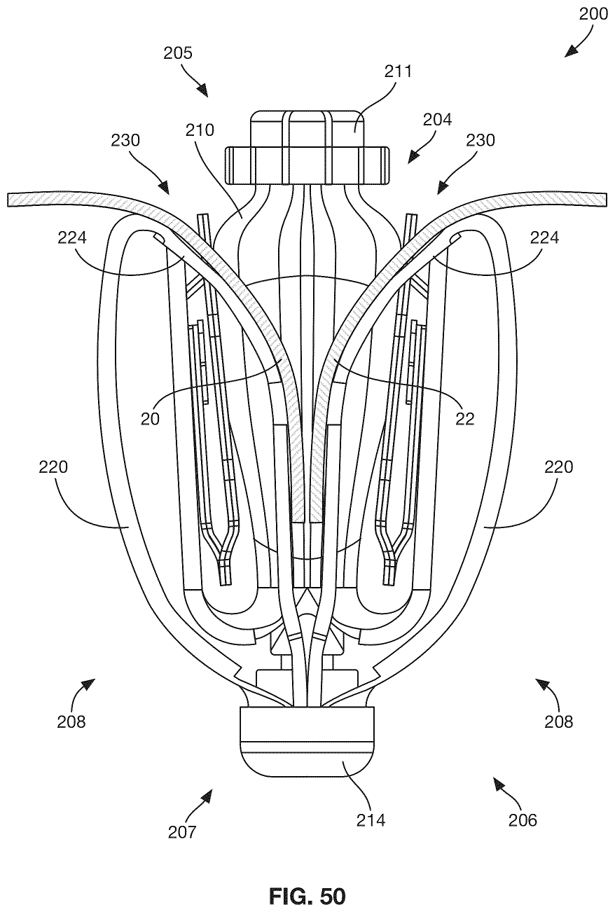

Referring to FIGS. 50-54, once the device 200 is implanted in a native valve, the coaptation element 210 functions as a gap filler in the valve regurgitant orifice, such as the gap 26 in the mitral valve MV illustrated by FIG. 6 or a gap in another native valve. In some implementations, when the device 200 has been deployed between the two opposing valve leaflets 20, 22, the leaflets 20, 22 no longer coapt against each other in the area of the coaptation element 210, but instead coapt against the coaptation element 210. This reduces the distance the leaflets 20, 22 need to be approximated to close the mitral valve MV during systole, thereby facilitating repair of functional valve disease that may be causing mitral regurgitation. A reduction in leaflet approximation distance can result in several other advantages as well. For example, the reduced approximation distance required of the leaflets 20, 22 reduces or minimizes the stress experienced by the native valve. Shorter approximation distance of the valve leaflets 20,22 can also require less approximation forces which can result in less tension experienced by the leaflets 20, 22 and less diameter reduction of the valve annulus. The smaller reduction of the valve annulus—or none at all—can result in less reduction in valve orifice area as compared to a device without a coaptation element or spacer. In this way, the coaptation element 210 can reduce the transvalvular gradients.

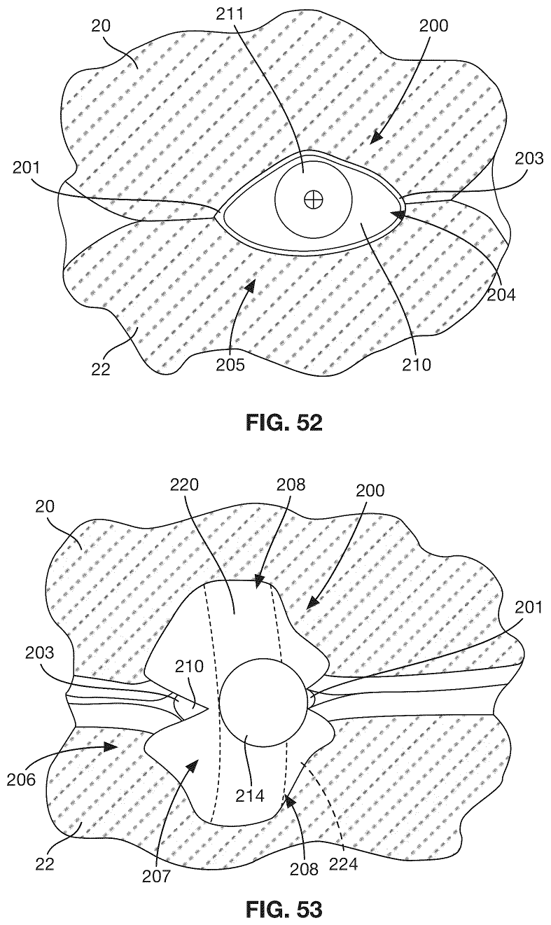

To adequately fill the gap 26 between the leaflets 20, 22, the device 200 and the components thereof can have a wide variety of different shapes and sizes. For example, the outer paddles 220 and paddle frames 224 can be configured to conform to the shape or geometry of the coaptation element 210 as is shown in FIGS. 50-54. As a result, the outer paddles 220 and paddle frames 224 can mate with both the coaptation element 210 and the native valve leaflets 20, 22. In some implementations, when the leaflets 20, 22 are coapted against the coaptation element 210, the leaflets 20, 22 fully surround or “hug” the coaptation element 210 in its entirety, thus small leaks at lateral and medial aspects 201, 203 of the coaptation element 210 can be prevented. The interaction of the leaflets 20, 22 and the device 200 is made clear in FIG. 51, which shows a schematic atrial or surgeon's view that shows the paddle frame 224 (which would not actually be visible from a true atrial view, e.g., FIG. 52), conforming to the coaptation element 210 geometry. The opposing leaflets 20, 22 (the ends of which would also not be visible in the true atrial view, e.g., FIG. 52) being approximated by the paddle frames 224, to fully surround or “hug” the coaptation element 210.

This coaptation of the leaflets 20, 22 against the lateral and medial aspects 201, 203 of the coaptation element 210 (shown from the atrial side in FIG. 52, and the ventricular side in FIG. 53) would seem to contradict the statement above that the presence of a coaptation element 210 minimizes the distance the leaflets need to be approximated. However, the distance the leaflets 20, 22 need to be approximated is still minimized if the coaptation element 210 is placed precisely at a regurgitant gap 26 and the regurgitant gap 26 is less than the width (medial-lateral) of the coaptation element 210.

FIG. 50 illustrates the geometry of the coaptation element 210 and the paddle frame 224 from an LVOT perspective. As can be seen in this view, the coaptation element 210 has a tapered shape being smaller in dimension in the area closer to where the inside surfaces of the leaflets 20, 22 are required to coapt and increase in dimension as the coaptation element 210 extends toward the atrium. Thus, the depicted native valve geometry is accommodated by a tapered coaptation element geometry. Still referring to FIG. 50, the tapered coaptation element geometry, in conjunction with the illustrated expanding paddle frame 224 shape (toward the valve annulus) can help to achieve coaptation on the lower end of the leaflets, reduce stress, and minimize transvalvular gradients.

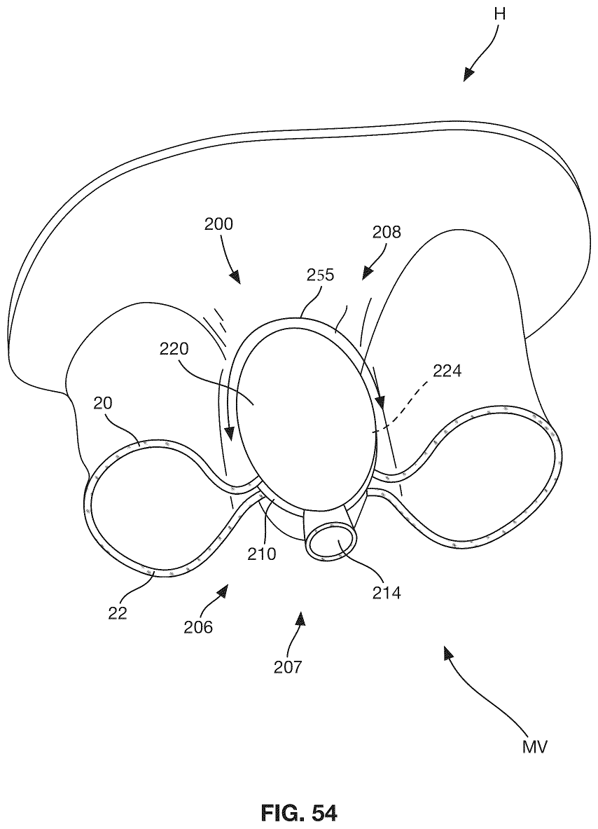

Referring to FIG. 54, the shape of the coaptation element 210 and the paddle frames 224 can be defined based on an Intra-Commissural view of the native valve and the device 200. Two factors of these shapes are leaflet coaptation against the coaptation element 210 and reduction of stress on the leaflets due to the coaptation. Referring to FIGS. 54 and 24, to both coapt the valve leaflets 20, 22 against the coaptation element 210 and reduce the stress applied to the valve leaflets 20, 22 by the coaptation element 210 and/or the paddle frames 224, the coaptation element 210 can have a round or rounded shape and the paddle frames 224 can have a full radius that spans nearly the entirety of the paddle frame 224. The round shape of the coaptation element 210 and/or the illustrated fully rounded shape of the paddle frames 224 distributes the stresses on the leaflets 20, 22 across a large, curved engagement area 255. For example, in FIG. 54, the force on the leaflets 20, 22 by the paddle frames is spread along the entire rounded length of the paddle frame 224, as the leaflets 20 try to open during the diastole cycle.

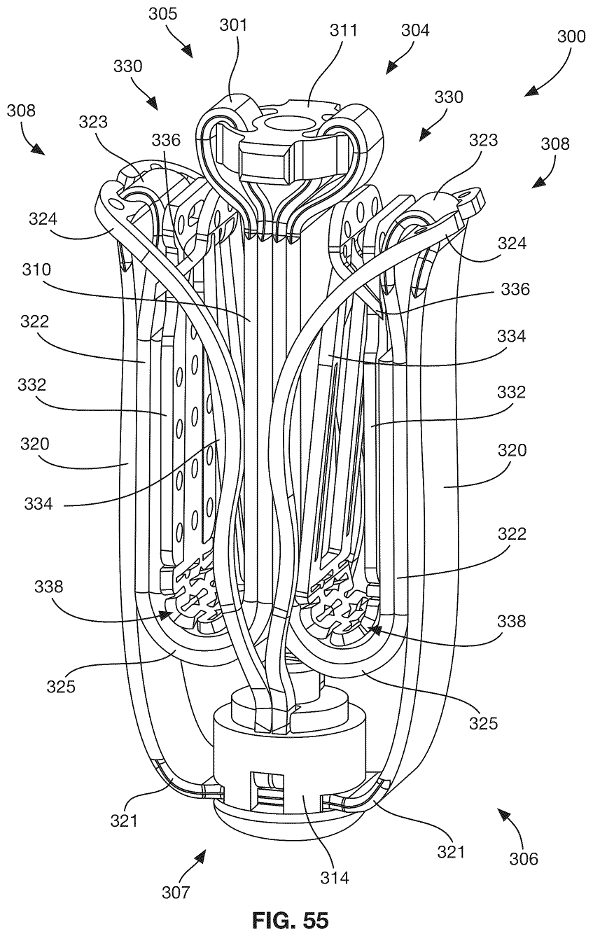

Referring now to FIG. 55, an example of an implantable device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism (e.g., a capture mechanism such as the capture mechanism 213 shown in FIGS. 43-49) of a delivery system (e.g., a delivery system such as the system shown in FIGS. 38-42 and 49).

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation member or coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation member or coaptation element 310, the coaptation member or coaptation element 310 and the anchors 308 can be coupled together in various ways. For example, as shown in the illustrated implementation, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from the continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.) and thus the anchors 308 move relative to a midpoint of the device. This movement can be along a longitudinal axis extending between the distal end (e.g., cap 314, etc.) and the proximal end (e.g., collar 311 or other attachment element, etc.) of the device. For example, the anchors 308 can be positioned in a fully extended or straight configuration (e.g., similar to the configuration of device 200 shown in FIG. 36) by moving the distal end (e.g., cap 314, etc.) away from the proximal end of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the coaptation element 310 (e.g., similar to the configuration of device 200 shown in FIG. 36). From the straight configuration, the anchors 308 can be moved to a fully folded configuration (e.g., FIG. 55), e.g., by moving the proximal end and distal end toward each other and/or toward a midpoint or center of the device. Initially, as the distal end (e.g., cap 314, etc.) moves toward the proximal end and/or midpoint or center of the device, the anchors 308 bend at connection portions 321, 323, 325, and the connection portions 323 move radially outwardly relative to the longitudinal axis of the device 300 and axially toward the midpoint and/or toward the proximal end of the device (e.g., similar to the configuration of device 200 shown in FIG. 34). As the cap 314 continues to move toward the midpoint and/or toward the proximal end of the device, the connection portions 323 move radially inwardly relative to the longitudinal axis of the device 300 and axially toward the proximal end of the device (e.g., similar to the configuration of device 200 shown in FIG. 30).

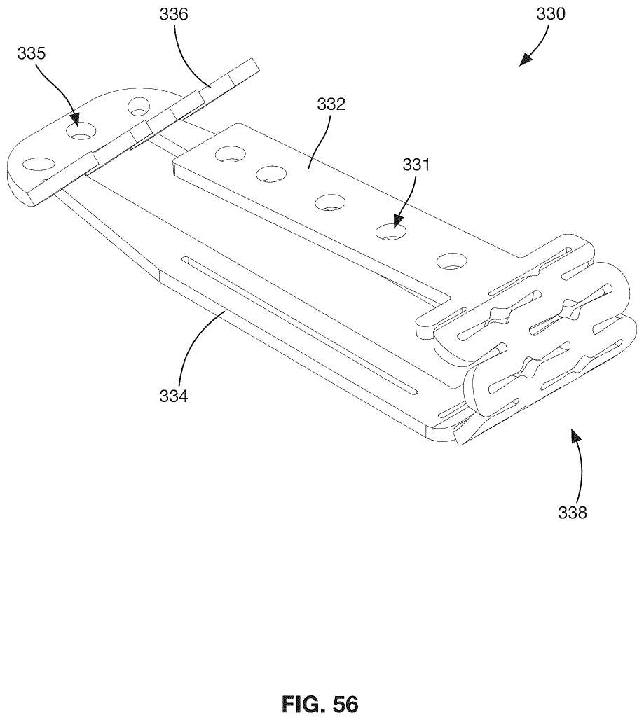

In some implementations, the clasps comprise a moveable arm coupled to an anchor. In some implementations, the clasps 330 (as shown in detail in FIG. 56) include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots 331 with sutures (not shown). The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the barbs 336. The clasps 330 are opened by applying tension to actuation lines (e.g., the actuation lines 216 shown in FIGS. 43-48) attached to holes 335 in the moveable arms 334, thereby causing the moveable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 55 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 55, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

During implantation of an implantable device or implant in the native heart valve, movement of the device to the implanted position may be impeded or obstructed by the native heart structures. For example, articulable portions of an implantable device or implant (such as paddle portions of anchors used to secure the device to the native heart valve tissue) may rub against, become temporarily caught, or be temporarily blocked by the chordae tendineae CT (shown in FIGS. 3 and 4) that extend to the valve leaflets. An example implantable device or implant can be configured to reduce the likelihood of the device or implant getting temporarily caught or blocked by the CT. For example, the implantable device or implant can take a wide variety of different configurations that are configured to be actively or passively narrowed to reduce the width of a paddle frame of an anchor portion of the device and, consequently, reduce the surface area of the device, which will make it easier to move the device/implant past and/or through the CT.

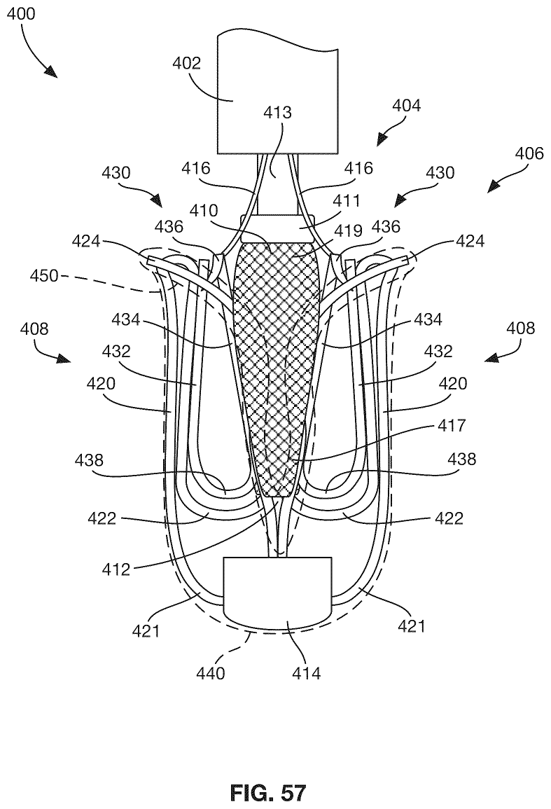

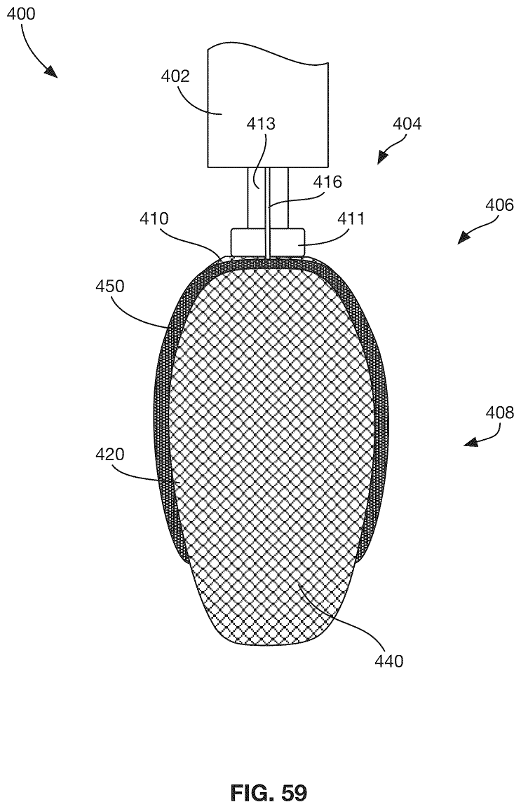

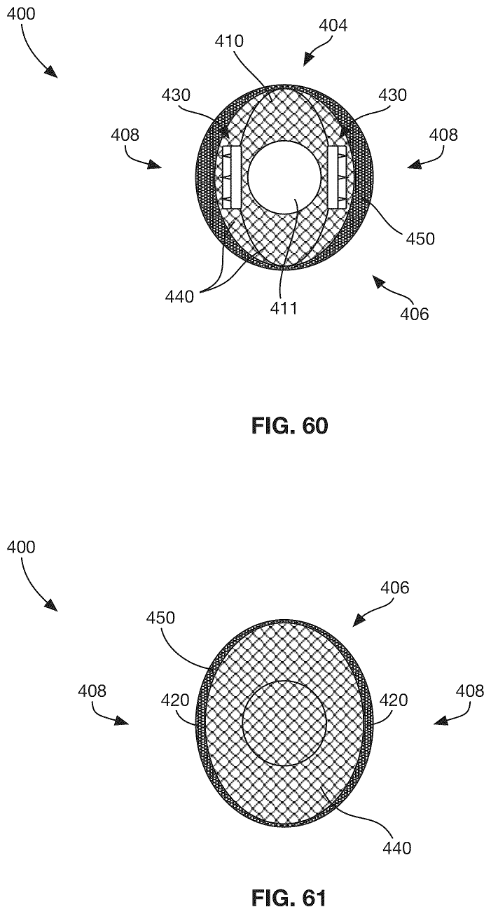

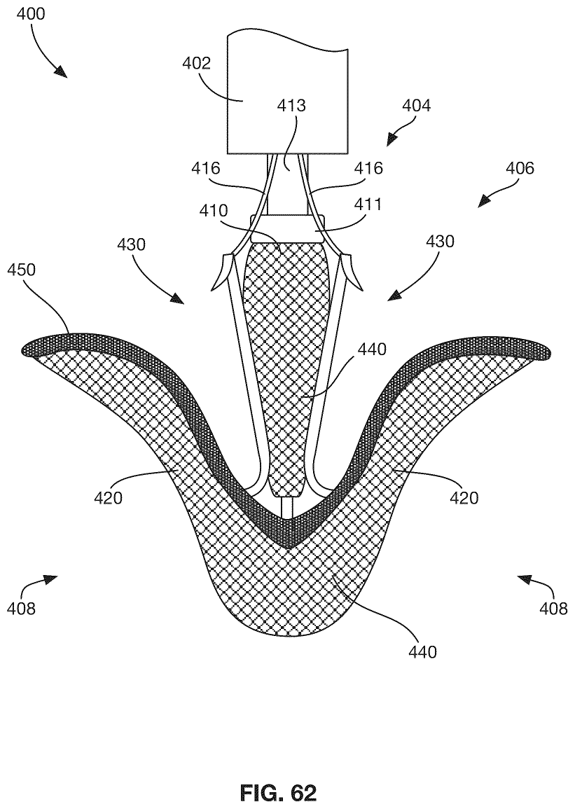

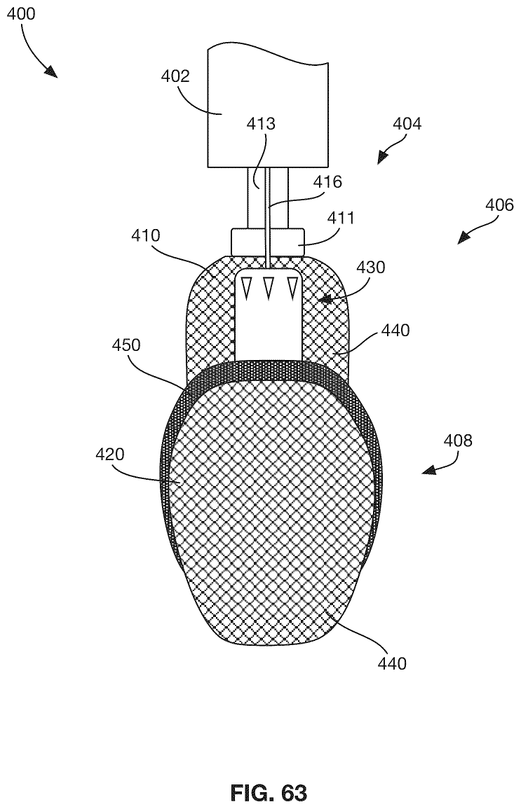

Referring now to FIGS. 57-67, an example implementation of an implantable device or implant 400 is shown. The device 400 includes materials and/or coatings that create a more lubricious or slippery or smooth exterior surface to reduce friction due to engagement between the native structures of the heart—e.g., chordae—and the device 400. This reduction in friction allows the device 400 to maneuver more easily into position for implantation in the heart. The device 400 can include any other features for an implantable device or implant discussed in the present application or in the applications and patents incorporated by reference herein, and the device 400 can be positioned to engage valve tissue 20, 22 as part of any suitable valve repair system (e.g., any valve repair system disclosed in the present application or any currently known valve repair system). In addition, any of the devices described herein can incorporate the features of the device 400.

Referring now to FIG. 57, the implantable device or implant 400 can be deployed from a delivery sheath or means for delivery 402 by a pusher 413, such as a rod or tube as described above. The device 400 can include a coaptation portion 404 and the anchor portion 406. The anchor portion 406 can including two or more anchors 408.

The coaptation portion 404 can optionally include a coaptation element or spacer 410. The anchor portion 406 includes a plurality of paddles 420 (e.g., two in the illustrated implementation), and a plurality of clasps 430 (e.g., two in the illustrated implementation).

A first or proximal collar 411, and a second collar or cap 414 are used to move the coaptation portion 404 and the anchor portion 406 relative to one another. Actuation of the actuator, actuation element, or means for actuating 412 opens and closes the anchor portion 406 of the device 400 to grasp the native valve leaflets during implantation in the manner described above. The actuator or actuation element 412 can take a wide variety of different forms. For example, the actuation element can be threaded such that rotation of the actuation element moves the anchor portion 406 relative to the coaptation portion 404. Or, the actuation element can be unthreaded, such that pushing or pulling the actuation element 412 moves the anchor portion 406 relative to the coaptation portion 404.

The coaptation element 410 extends from a proximal portion 419 assembled to the collar 411 to a distal portion 417 that connects to the anchors 408. The coaptation element 410 and the anchors 408 can be coupled together in various ways. For example, as shown in the illustrated implementation, the coaptation element 410 and the anchors 408 can optionally be coupled together by integrally forming the coaptation element 410 and the anchors 408 as a single, unitary component. This can be accomplished, for example, by forming the coaptation element 410 and the anchors 408 from a continuous strip of a braided or woven material, such as braided or woven nitinol wire. In another implementation, the components are separately formed and are attached together.

The anchors 408 are attached to the coaptation element 410 by inner flexible portions 422 and to the cap 414 by outer flexible portions 421. The anchors 408 can comprise a pair of paddle portions 420. In some implementations, the anchors 408 can comprise inner and outer paddles joined by a flexible portion (e.g., the paddles 220, 222 of the device 200 joined by hinge portion 223). The paddle portions 420 are attached to paddle frames 424 that are flexibly attached to the cap 414.

Like the device 200 shown in FIGS. 22-37, the anchors 408 can be configured to move between various configurations by axially moving the cap 414 relative to the proximal collar 411 and thus the anchors 408 relative to the coaptation element 410 along a longitudinal axis extending between the cap 414 and the proximal collar 411. For example, the anchors 408 can be positioned in a straight configuration by moving the cap 414 away from the coaptation element 410. The anchors 408 can also be positioned in a closed configuration (e.g., FIG. 57) by moving the cap 414 toward the coaptation element 410. When the cap 414 is pulled all the way toward the coaptation element 410 by the actuation element 412, the paddle portions 420 are closed against the coaptation element 410 and any native tissue (e.g., a valve leaflet, not shown) captured between the coaptation element 410 and the paddle portion 420 is pinched so as to secure the device 400 to the native tissue.

The clasps 430 can comprise attachment or fixed portions 432 that are hingeably connected to arm or moveable portions 434 by hinge portions 438. The moveable portions 434 can include barbs or means for securing 436 that can pierce the native leaflets to further secure native leaflets captured between the fixed and moveable portions 432, 434 of the clasps 430. The attachment or fixed portions 432 can be coupled or connected to the paddle portions 420 of the anchors 408 in various ways such as with sutures, adhesive, fasteners, welding, stitching, swaging, friction fit and/or other means for coupling. The clasps 430 can be similar to or the same as the clasps 430.

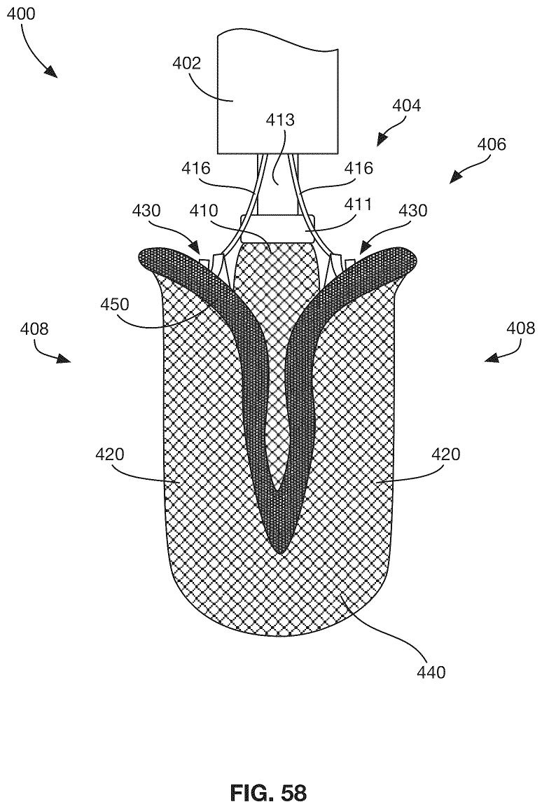

The moveable portions 434 can articulate, pivot, and/or flex relative to the fixed portions 432 between open configurations (e.g., like the device 200 shown in FIGS. 30-37) and a closed configuration (e.g., FIGS. 57-58). In some implementations, the clasps 430 can be biased to the closed configuration. In the open configuration, the fixed portions 432 and the moveable portions 434 articulate, pivot, and/or flex away from each other such that native leaflets (see, e.g., FIGS. 38-49) can be positioned between the fixed portions 432 and the moveable portions 434. In the closed configuration, the fixed portions 432 and the moveable portions 434 articulate, pivot, and/or flex toward each other, thereby clamping the native leaflets between the fixed portions 432 and the moveable portions 434 (e.g., FIG. 47). The clasps 430 can be spring loaded so that in the closed position the clasps 430 continue to provide a pinching force on the grasped native leaflet. This pinching force remains constant regardless of the position of the paddle portions 420.

Tension can be applied to actuation lines 416 connected to the clasps 430 to pull the moveable portions 434 of the clasps 430 in the retracting or proximal direction while the paddle portions 420 remain opened as described above. The clasps 430 are opened against the biasing force of the hinge portions 438 described above. Once the clasps 430 are opened, the device 400 is moved in a capture direction by retracting the pusher tube or rod 413 into the catheter 402 and/or moving the catheter 402 to position the leaflets 20, 22 between the fixed portions 432 and the moveable portions 434 of the open clasps 430. Once the device 400 is in position to capture the leaflets 20, 22, tension on the actuation lines 416 is released, thereby allowing the actuation lines 416 to move in a releasing direction so that the spring-loaded hinge portions 438 cause the clasps 430 to close as described above to capture and pinch the leaflets 20, 22 between the fixed and moveable portions 432, 434 of the clasps 430.

Referring now to FIGS. 58-71, the implantable device or implant 400 is shown with portions of the device 400 covered by a first cover portion 440 and a second cover portion 450. The first cover portion 440 provides for ingrowth of the native heart tissue to improve the connection between the native heart tissue and the device 400, while the second cover portion 450 provides a more lubricious or slippery surface to improve maneuverability of the device 400 during the implantation procedure. That is, the second cover portion 450 provides the device 400 with a surface having a lower friction coefficient than the first cover portion 440 so that the device 400 moves more easily against and/or past the native heart structures, such as the chordae. The second cover portion 450 can also be made from a material that both promotes tissue ingrowth, e.g., because of the thickness of the material, the size of the openings, and provides a low friction surface.

The first cover portion 440 is formed from a flexible material that promotes tissue ingrowth to further secure the implantable device/implant 400 between the native leaflets over time. The first cover portion 440 can be formed from fabric, cloth, or any other flexible material suitable for implantation in the human body.

As can be seen in FIGS. 57-60, 62-64, 66, 68, and 70 the first cover portion 440 is formed around the coaptation element 410 and the paddle portions 420. The first cover portion 440 can also extend to cover portions of the clasps 430. Thus, once the native leaflets have been captured by the device 400, the areas of the leaflets in contact with the first cover portion 440 are able to grow into the material of the first cover portion 440 to enhance the grip of the anchors 408 on the leaflets.

The second cover portion 450 can be a section of the first cover portion 440 that is treated to provide a lower coefficient of friction or can be formed from a different material that is joined to the first cover portion 450, either at a seam or by overlaying a piece of material forming the second cover portion 450 on top of the first cover portion 440. The first and second cover portions 440, 450 can be joined together in any suitable way, such as, for example, by sewing, with an adhesive, by coating, with a thermal bonding layer, or the like.

The first cover portion 440 and the second cover portion 450 can take a wide variety of different forms. In various different example implementations, the second, lower coefficient of friction portion can be provided at portions of the device 400 that are likely to engage native internal structures of the heart during advancement, positioning, and/or implantation of the device within the heart. The second, lower coefficient of friction portion can be included on anchor portion(s), such as on the illustrated paddles and/or clasps. In some implementations, the anchor portion(s) can take other forms that may or may not include paddles and clasps. The second, lower coefficient of friction portion can be included on a coaptation portion of the device. In some implementations, the second, lower coefficient of friction portion is not included on a coaptation portion or the device does include a coaptation portion.

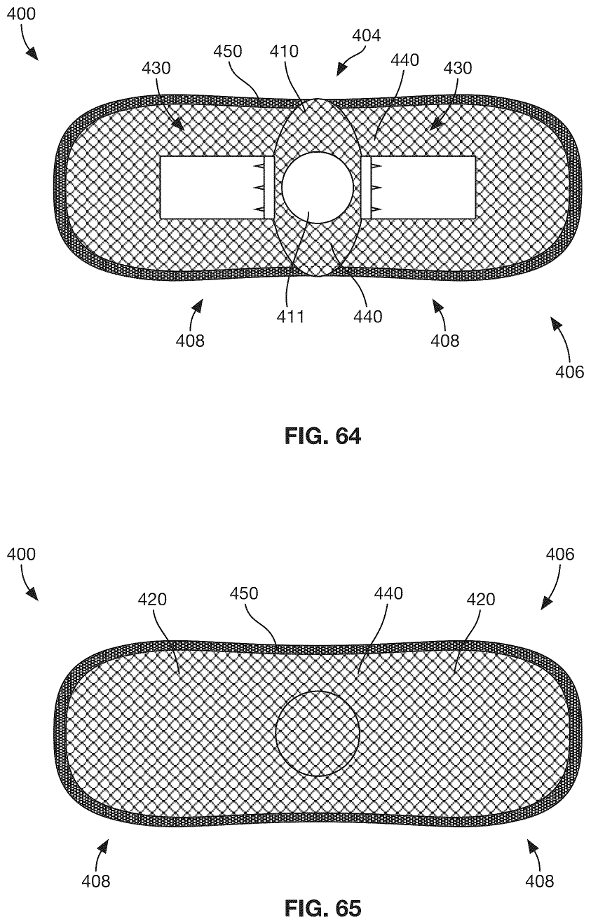

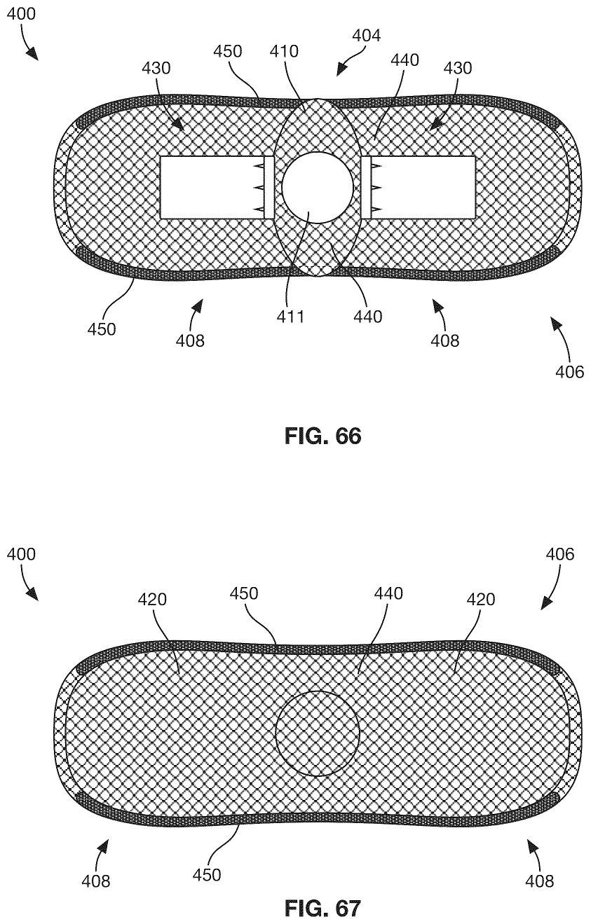

The second cover portion 450 can cover a portion or all of the edges of the paddle portions 420, as shown in FIGS. 58-71. For example, the second cover portion 450 illustrated by FIGS. 64-65 covers all of the edge portion while the second cover portion 450 does not cover the ends of the paddle portions 420 in FIGS. 66-67. Providing an increased friction portion at the ends of the paddle portions 420 facilitates an increased friction or gripping force against the native leaflets during capture and maintaining a lower friction area on the sides of the paddle portions 420 helps to avoid entanglement with the chordae during implantation.

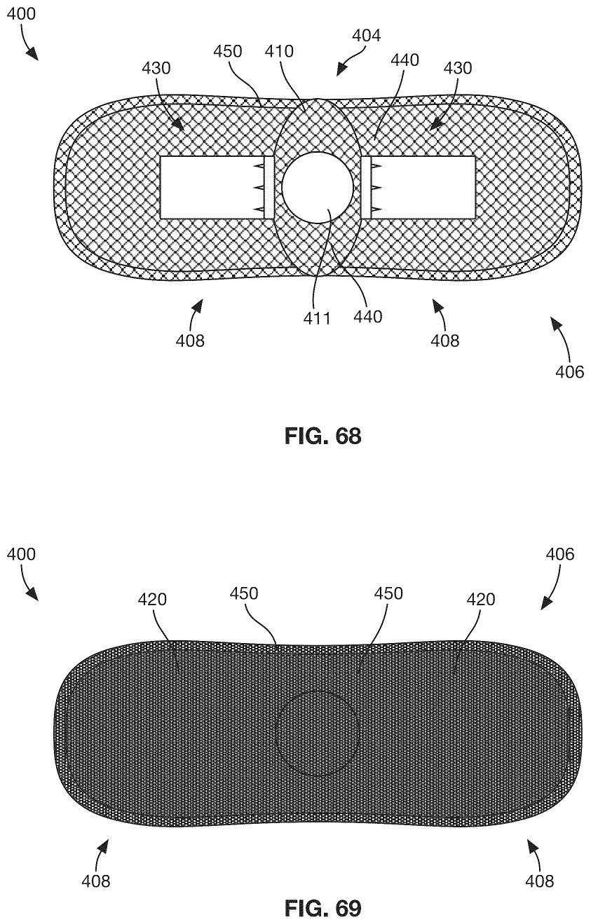

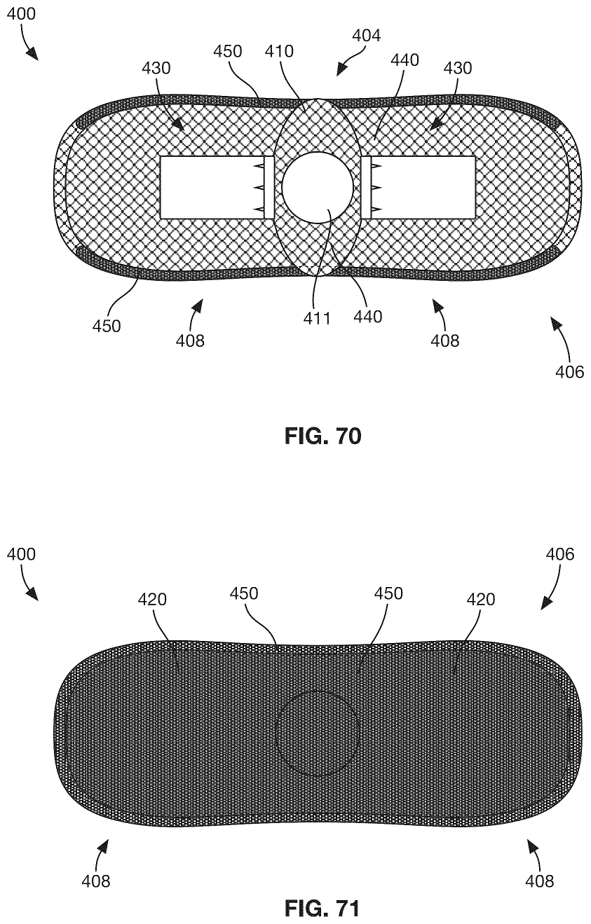

In some implementations, the second cover portion 450 illustrated by FIGS. 58-71 can be extended to cover a portion of or all of the external surfaces of the paddle portion 420. For example, the second cover portion 450 could be extended to cover all of the first cover portion 440 shown in FIG. 65. FIGS. 68-71 illustrate an example implementation that is similar to the configuration of FIGS. 64-67 where a proximal side of the paddles has the first cover portion 440 and a distal side of the paddles has the second cover portion 450. FIGS. 70-71 are similar to the configuration of FIGS. 66-67 where the second cover portion 450 does not extend to cover the ends of the paddle portions 420 on the top side of the device 400.

The second cover portion 450 can take on a wide variety of forms to provide a lower coefficient of friction between the device 400 and the native tissues in the heart. The second cover portion 450 can be formed from a fabric material that has a lower coefficient of friction than that of the first cover portion 440. That is, the second cover portion 450 can be formed from a fabric woven from threads of a different material than the first cover portion 440. The second cover portion 450 can also be formed by embedding materials, such as plastic particles, into an area of the first cover portion 440 or by applying a coating to an area of the first cover portion 440.

Coatings applied to the second cover portion 450 can be permanent coatings or can be temporary coatings that dissolve in blood after one or more hours, such as between one hour and one year, such as between one hour and six months, such as between one hour and three months, such as between one hour and one month, such as between one hour and two weeks, such as between one hour and one week. Temporary coatings provide the desired decreased friction during the implantation procedure and then dissolve so that more of the first cover portion 440 is exposed and can contact the native tissue to provide additional gripping surface area. The coating forming the second cover portion 450 could be applied during manufacturing of the device 400 or could be applied by the person performing the implantation procedure.

In an example implementation, the second cover portion 450 is formed from a hydrophilic material, includes or incorporates a hydrophilic material, or is coated with a hydrophilic coating. Hydrophilic materials and coatings reduce surface friction of medical devices and increase the lubricity or slipperiness of the surface of the device to which the material is added. Hydrophilic materials readily absorb liquid like a microscopic sponge to provide a low friction surface as long as the material remains wet.

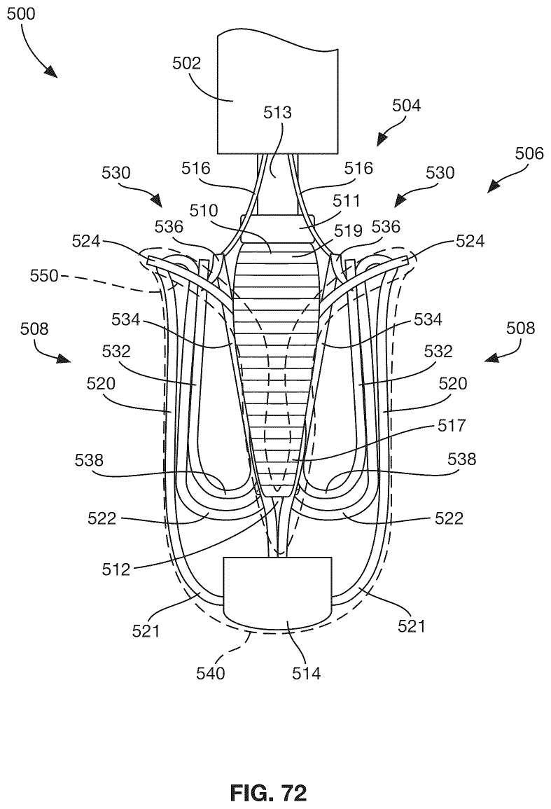

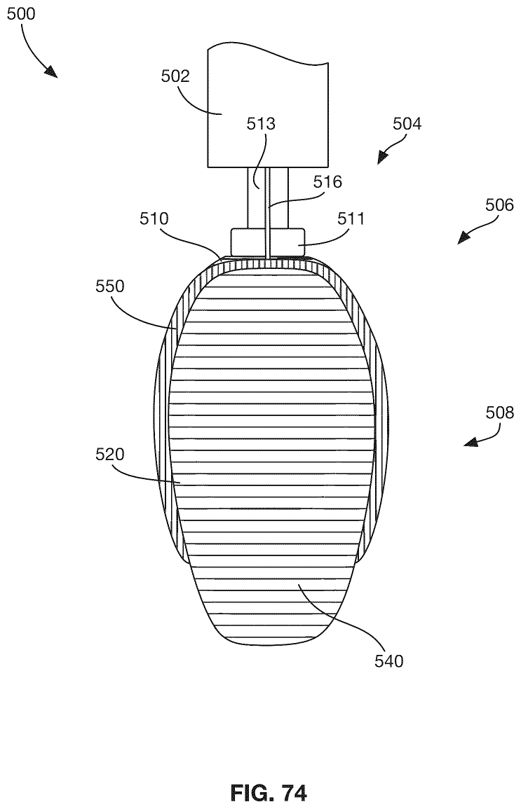

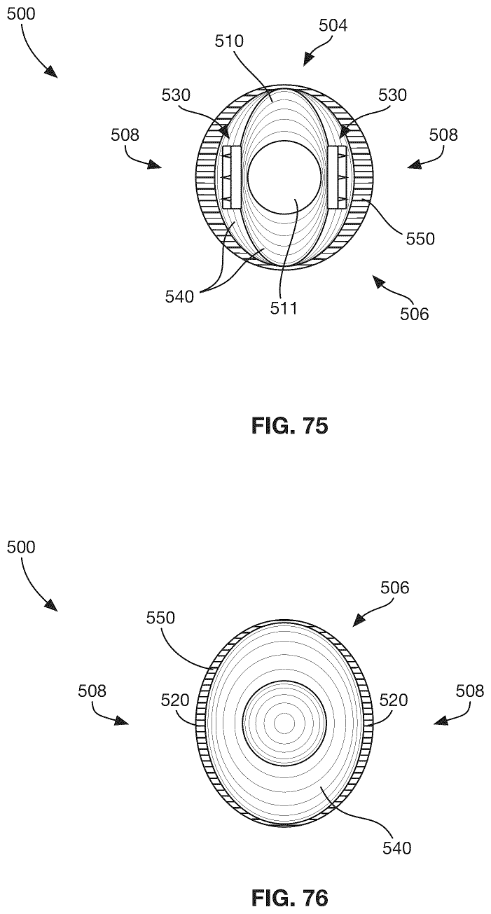

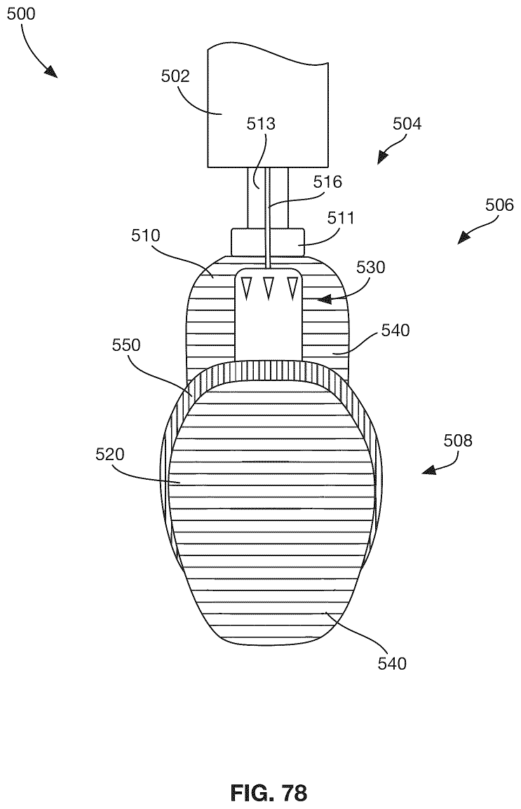

Referring now to FIGS. 72-86, an example implementation of an implantable device or implant 500 is shown. The device 500 includes materials and/or coatings that include surface features that create an exterior surface with reduced friction due to engagement between the native structures of the heart—e.g., the chordae tendineae—and the device 500. This reduction in friction allows the device 500 to maneuver more easily into position for implantation in the heart. The device 500 can include any other features for an implantable device/implant discussed in the present application or in the applications and patents incorporated by reference herein, and the device 500 can be positioned to engage valve tissue 20, 22 as part of any suitable valve repair system (e.g., any valve repair system disclosed in the present application or any currently known valve repair system). In addition, any of the devices described herein can incorporate the features of the device 500.

Referring now to FIG. 72, the spacer or coaptation device 500 can be deployed from a delivery sheath or means for delivery 502 by a pusher 513, such as a rod or tube as described above. The device 500 can include a coaptation portion 504 and the anchor portion 506. The anchor portion 506 can include two or more anchors 508.

For some applications, the coaptation portion 504 can include an optional coaptation element or spacer 510. The anchor portion 506 includes a plurality of paddles 520 (e.g., two in the illustrated implementation), and a plurality of clasps 530 (e.g., two in the illustrated implementation).

A first or proximal collar 511, and a second collar or cap 514 are used to move the coaptation portion 504 and the anchor portion 506 relative to one another. Actuation of the actuator, actuation element or means for actuating 512 opens and closes the anchor portion 506 of the device 500 to grasp the native valve leaflets during implantation in the manner described above. The actuator or actuation element 512 can take a wide variety of different forms. For example, the actuation element can be threaded such that rotation of the actuation element moves the anchor portion 506 relative to the coaptation portion 504. Or, the actuation element can be unthreaded, such that pushing or pulling the actuation element 512 moves the anchor portion 506 relative to the coaptation portion 504.

The coaptation element 510 extends from a proximal portion 519 assembled to the collar 511 to a distal portion 517 that connects to the anchors 508. The coaptation element 510 and the anchors 508 can be coupled together in various ways. For example, as shown in the illustrated implementation, the coaptation element 510 and the anchors 508 can optionally be coupled together by integrally forming the coaptation element 510 and the anchors 508 as a single, unitary component. This can be accomplished, for example, by forming the coaptation element 510 and the anchors 508 from a continuous strip of a braided or woven material, such as braided or woven nitinol wire. In some implementations, the components are separately formed and are attached together.

The anchors 508 are attached to the coaptation element 510 by inner flexible portions 522 and to the cap 514 by outer flexible portions 521. The anchors 508 can comprise a pair of paddle portions 520. In some implementations, the anchors 508 can comprise inner and outer paddles joined by a flexible portion (e.g., the paddles 220, 222 of the device 200 joined by hinge portion 223). The paddle portions 520 are attached to paddle frames 524 that are flexibly attached to the cap 514.

Like the device 200 shown in FIGS. 22-37, the anchors 508 can be configured to move between various configurations by axially moving the cap 514 relative to the proximal collar 511 and thus the anchors 508 relative to the coaptation element 510 along a longitudinal axis extending between the cap 514 and the proximal collar 511. For example, the anchors 508 can be positioned in a straight configuration by moving the cap 514 away from the coaptation element 510. The anchors 508 can also be positioned in a closed configuration (e.g., FIG. 72) by moving the cap 514 toward the coaptation element 510. When the cap 514 is pulled all the way toward the coaptation element 510 by the actuation element 512, the paddle portions 520 are closed against the coaptation element 510 and any native tissue (e.g., a valve leaflet, not shown) captured between the coaptation element 510 and the paddle portion 520 is pinched so as to secure the device 500 to the native tissue.

The clasps 530 can comprise attachment or fixed portions 532 that are hingeably connected to arm or moveable portions 534 by hinge portions 538. The moveable portions 534 can include barbs or means for securing 536 that can pierce the native leaflets to further secure native leaflets captured between the fixed and moveable portions 532, 534 of the clasps 530. The attachment or fixed portions 532 can be coupled or connected to the paddle portions 520 of the anchors 508 in various ways such as with sutures, adhesive, fasteners, welding, stitching, swaging, friction fit and/or other means for coupling.

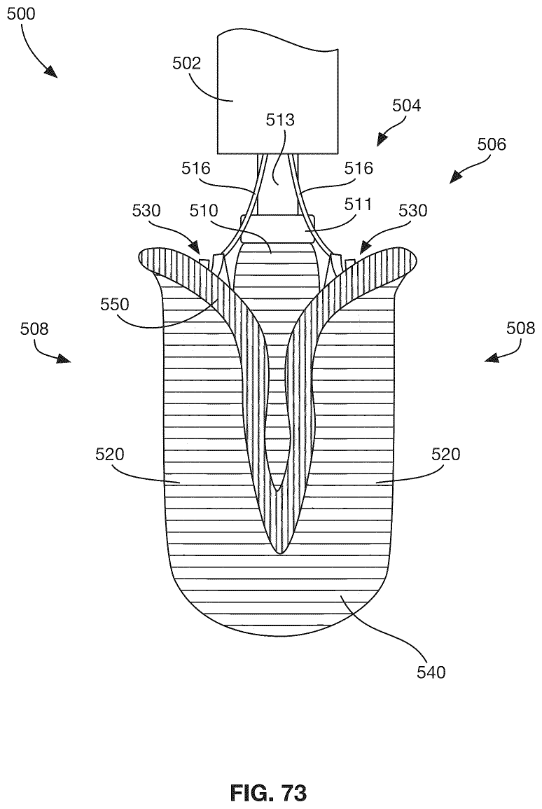

The moveable portions 534 can articulate, pivot, and/or flex relative to the fixed portions 532 between open configurations (e.g., like the device 200 shown in FIGS. 30-37) and a closed configuration (e.g., FIGS. 72-73). In some implementations, the clasps 530 can be biased to the closed configuration. In the open configuration, the fixed portions 532 and the moveable portions 534 articulate, pivot, and/or flex away from each other such that native leaflets (see, e.g., FIGS. 38-49) can be positioned between the fixed portions 532 and the moveable portions 534. In the closed configuration, the fixed portions 532 and the moveable portions 534 articulate, pivot, and/or flex toward each other, thereby clamping the native leaflets between the fixed portions 532 and the moveable portions 534 (e.g., FIG. 47). The clasps 530 can be spring loaded so that in the closed position the clasps 530 continue to provide a pinching force on the grasped native leaflet. This pinching force remains constant regardless of the position of the paddle portions 520.

Tension can be applied to actuation lines 516 connected to the clasps 530 to pull the moveable portions 534 of the clasps 530 in the retracting or proximal direction while the paddle portions 520 remain opened as described above. The clasps 530 are opened against the biasing force of the hinge portions 538 described above. Once the clasps 530 are opened, the device 500 is moved in a capture direction by retracting the pusher tube or rod 513 into the catheter 502 and/or moving the catheter 502 to position the leaflets 20, 22 between the fixed portions 532 and the moveable portions 534 of the open clasps 530. Once the device 500 is in position to capture the leaflets 20, 22, tension on the actuation lines 516 is released, thereby allowing the actuation lines 516 to move in a releasing direction so that the spring-loaded hinge portions 538 cause the clasps 530 to close as described above to capture and pinch the leaflets 20, 22 between the fixed and moveable portions 532, 534 of the clasps 530.

Referring now to FIGS. 72-86, the implantable device or implant 500 is shown with portions of the device 500 covered by a first cover portion 540 and a second cover portion 550. The first cover portion 540 provides for ingrowth of the native heart tissue to improve the connection between the native heart tissue and the device 500, while the second cover portion 550 provides a more lubricious or slippery surface to improve maneuverability of the device 500 during the implantation procedure. That is, the second cover portion 550 provides the device 500 with a surface having a lower friction coefficient than the first cover portion 540 so that the device 500 moves more easily against and/or past the native heart structures, such as the chordae. The second cover portion 550 can also be made from a material that both promotes tissue ingrowth, e.g., because of the thickness of the material and the size of the openings, and provides a low friction surface.

The first cover portion 540 is formed from a flexible material that promotes tissue ingrowth to further secure the implantable device/implant 500 between the native leaflets over time. The first cover portion 540 can be formed from fabric, cloth, or any other flexible material suitable for implantation in the human body.

As can be seen in FIGS. 72-75, 77-79, 81, 83, and 85 the first cover portion 540 is formed around the coaptation element 510 and the paddle portions 520. The first cover portion 540 can also extend to cover portions of the clasps 530. Thus, once the native leaflets have been captured by the device 500, the areas of the leaflets in contact with the first cover portion 540 are able to grow into the material of the first cover portion 540 to enhance the grip of the anchors 508 on the leaflets.

The second cover portion 550 can be a section of the first cover portion 540 that is formed with surface features that provide a lower coefficient of friction or can be formed from a different, low-friction material that is joined to the first cover portion 550, either at a seam or by overlaying a piece of material forming the second cover portion 550 on top of the first cover portion 540. The first and second cover portions 540, 550 can be joined together in any suitable way, such as, for example, by sewing, with an adhesive, by coating, with a thermal bonding layer, or the like.

The first cover portion 540 and the second cover portion 550 can take a wide variety of different forms. In some implementations, the second, lower coefficient of friction portion can be provided at portions of the device 500 that are likely to engage native internal structures of the heart during advancement, positioning, and/or implantation of the device within the heart. The second, lower coefficient of friction portion can be included on anchor portion(s), such as on the illustrated paddles and/or clasps. In some implementations, the anchor portion(s) can take other forms that may or may not include paddles and clasps. The second, lower coefficient of friction portion can be included on a coaptation portion of the device. In some implementations, the second, lower coefficient of friction portion is not included on a coaptation portion or the device doesn't include a coaptation portion.

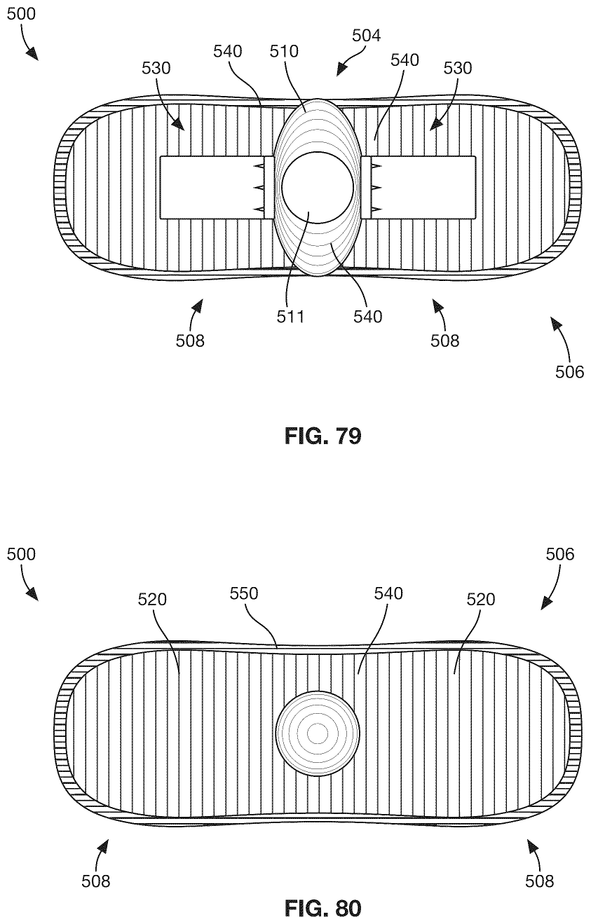

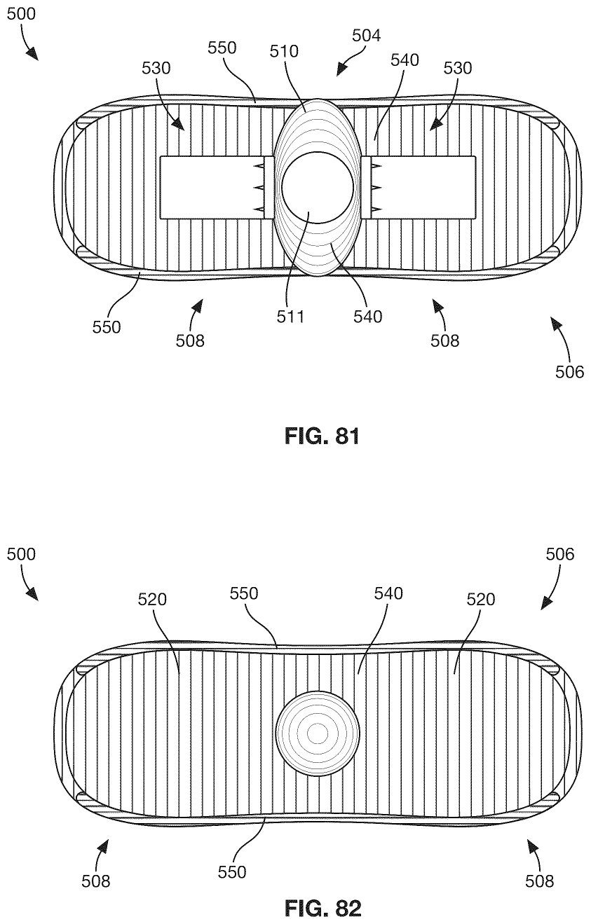

The second cover portion 550 can cover a portion or all of the edges of the paddle portions 520, as shown in FIGS. 73-86. For example, the second cover portion 550 illustrated by FIGS. 79-80 covers all of the edge portion while the second cover portion 550 does not cover the ends of the paddle portions 520 in FIGS. 81-82. Providing an increased friction portion at the ends of the paddle portions 420 facilitates an increased friction or gripping force against the native leaflets during capture and maintaining a lower friction area on the sides of the paddle portions 520 helps to avoid entanglement with the chordae during implantation. It should also be noted that the second cover portion 550 shown in FIGS. 79-80 provides surface features that change orientation relative to the leaflet as the leaflet moves along the edge of the paddle portion 520—the ridge portions are oriented along the edge on the sides of the paddle portions 520 and are oriented across the edge on the end portions of the paddle portions 520. The arrangement shown in FIGS. 81-82 provides surface features that are more closely aligned with the contour of the edges so that the leaflets and other tissues can slip past the paddle portions 520 unless in a capture-ready position.

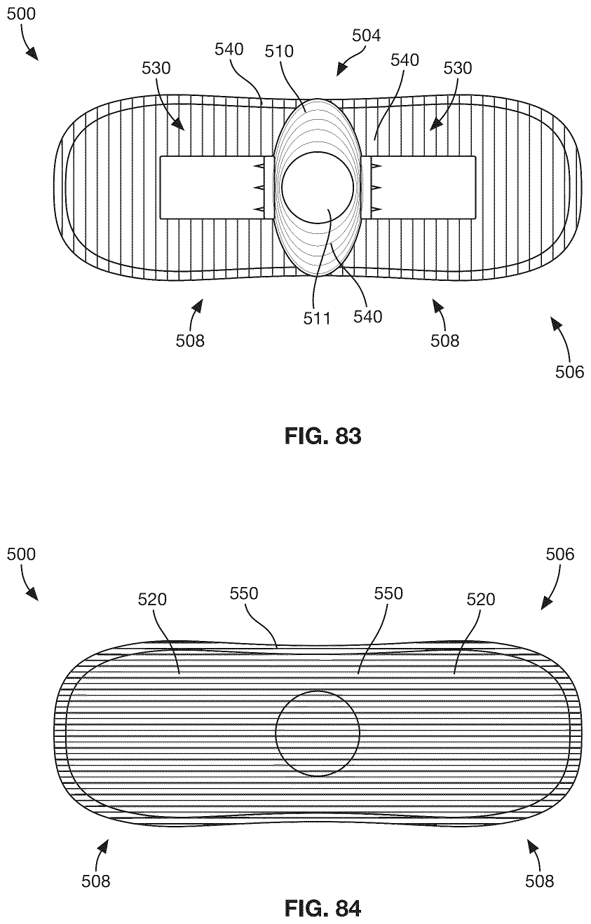

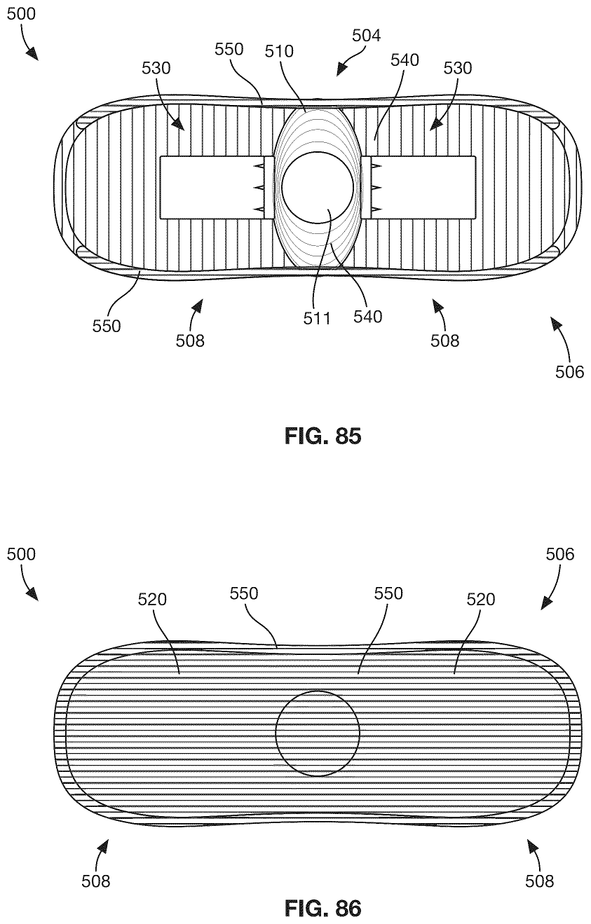

In some implementations, the second cover portion 550 illustrated by FIGS. 73-86 can be extended to cover a portion of or all of the external surfaces or outer portion of the paddle portion 520. For example, the second cover portion 550 could be extended to cover all of the first cover portion 540 shown in FIG. 80. FIGS. 83-86 illustrate an example implementation that is similar to the configuration of FIGS. 79-82 where a proximal side of the paddles has the first cover portion 540 and a distal side of the paddles has the second cover portion 550. FIGS. 85-86 are similar to the configuration of FIGS. 81-82 where the second cover portion 550 does not extend to cover the ends of the paddle portions 520 on the top side of the device 500.

The second cover portion 550 can take on a wide variety of forms to provide a lower coefficient of friction between the device 500 and the native tissues in the heart. The second cover portion 550 can be formed from the same material as the first cover portion 540 while being processed differently and/or can be oriented differently to create a lower coefficient of friction between the second cover portion 550 and the native tissue than between the first cover portion 540 and the native tissue.

In some implementations, the second cover portion 550 includes surface features that reduce the friction between the device 500 and the native heart tissue. For example, the second cover portion 550 can be formed with elongated ridges that are oriented in the direction of travel (see, e.g., FIG. 89) and thereby reduce friction with the native tissue and decrease the likelihood that a portion of the device 500 would catch on or otherwise be inhibited by the native tissue. The elongated ridges can be formed during or after the formation of the second cover portion 550. In some implementations, the ridges are formed as a result of the second cover portion 550 being knitted from a strand of material. For example, a knitting stitch could be used to form the second cover portion 550 wherein the wales of the knitted material are oriented in the longitudinal direction.

For example, the second cover portion 550 can be formed by knitting or weaving together the same strands of material used to form the first cover portion 540 but with a different knit or weave pattern or orientation to provide different surface properties in the second cover portion 550 than the first cover portion 540. In some implementations, the first cover portion 540 can have the wales of a knitted material or warp strands of a woven material oriented circumferentially while the second cover portion 550 can have the wales or warp strands oriented orthogonally to the wales or warp strands of the first cover portion 540—i.e., longitudinally or along the direction of movement of the device 500 during implantation. Example cover materials and their interactions with the native heart tissue are discussed in greater detail below.

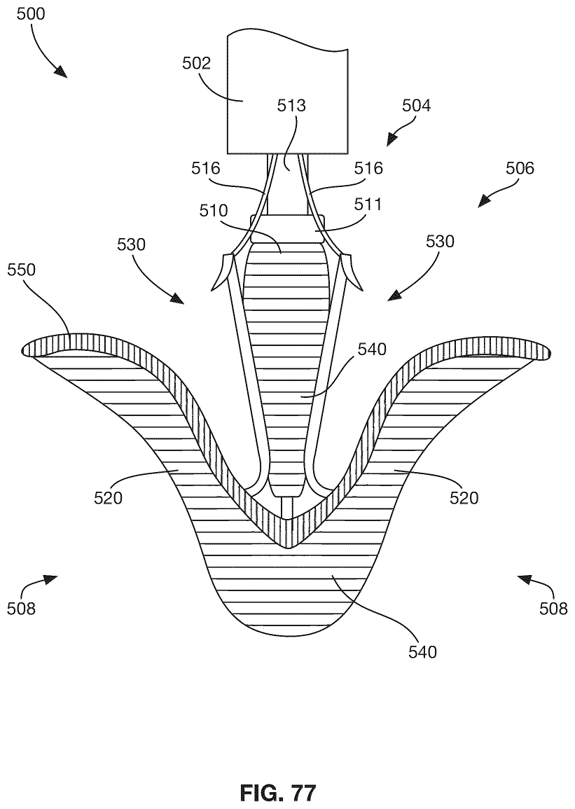

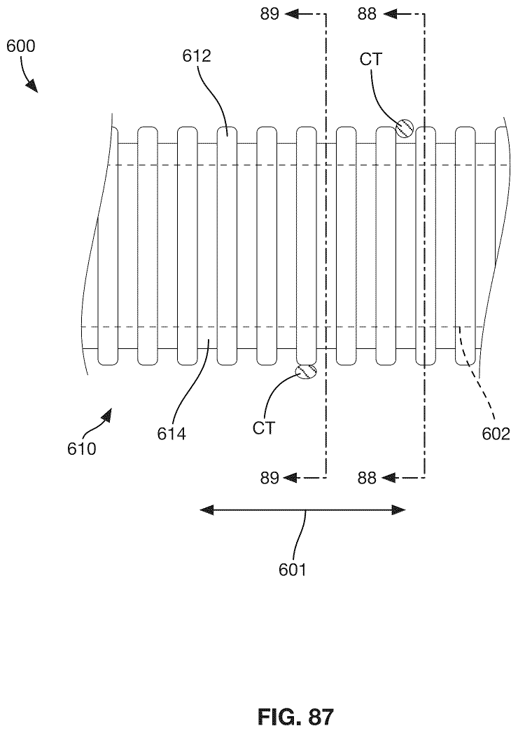

Referring now to FIGS. 87-92, abstract representations of implantable devices with covers having circumferentially or laterally oriented and longitudinally oriented surface features are shown interacting with the chordae tendineae CT. As used herein, the orientation terms “circumferential” or “lateral” describe directions that run generally or substantially across the width of the example implantable devices described herein, when those devices are viewed from the front or side, such as, for example, the device 500 in FIG. 73 or 77. A “circumferential” or “lateral” orientation can also be orthogonal to or askew to a direction of movement of the device.

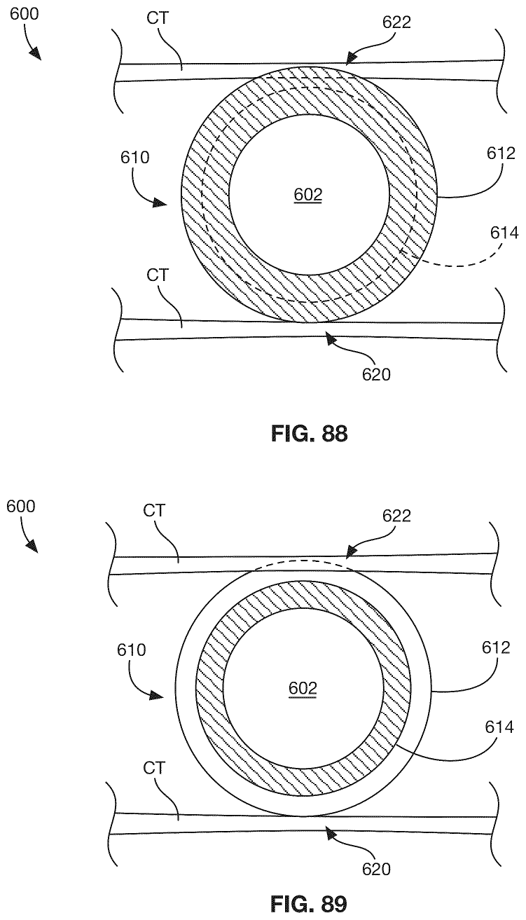

FIG. 87 is a side view of a portion of a cover 610 on a portion of an implantable device or implant 600, which can be any of the valve repair devices shown and described herein or any other known valve repair device. The device 600 is shown arranged between two chordae tendineae CT. In FIG. 87, the device 600 can travel in the direction indicated by double arrow 601, with the chordae tendinea extending into or out of the page. FIGS. 88 and 89 are cross-sectional views taken along the planes indicated by lines 88-88 and 89-89 in FIG. 87 respectively. As such, the view illustrated in FIG. 88 is a cross-sectional view of the device 600 through one of the ridge portions 612 of the cover material and FIG. 89 is a cross-sectional view of the device 600 through one of the valley portions 614 of the cover material. In FIGS. 88 and 89, the direction of travel 601 of the device 600 would be in or out of the plane of the page so that the device 600 would be moved in a direction 601 that is orthogonal to the length of the chordae tendineae CT.

The device 600 includes a device body 602 that is covered by a cover 610. The cover 610 includes ridge portions 612 having a major outer diameter spaced apart by valley portions 614 having a minor outer diameter. One of the chordae tendineae CT is in contact with the ridge portion 612 at a first contact area 620 while the other chordae tendineae CT is disposed within a valley portion 614 and is overlapping the neighboring ridge portions 612 at a second contact area 622. As the device 600 moves against the chordae tendineae CT, the first contact area 620 tends to increase in size because the ridge portions 612 are oriented in the same direction as the chordae tendineae CT and pressure applied by the ridge portions 612 against the chordae tendineae CT tends to wrap the chordae tendineae around the ridge portion 612. Further movement can cause the chordae tendineae CT to move into one of the valley portions 614 and overlap the ridge portions 612 as is shown in the second contact area 622. In some situations, it has been found that lower tension chordae tendineae CT are more likely to be captured within the valley portions 614. The force required to move the device 600 once the chordae tendineae CT has moved into the valley portion 614 increases significantly because the chordae tendineae CT is now caught on one of the neighboring ridge portion 612. In this way, both of the first and second contact areas 620, 622 lead to increased friction between the chordae tendineae CT and the device 600.

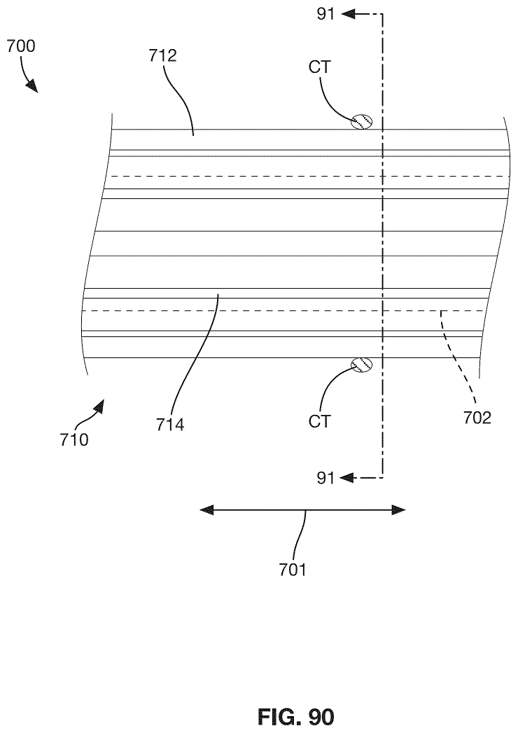

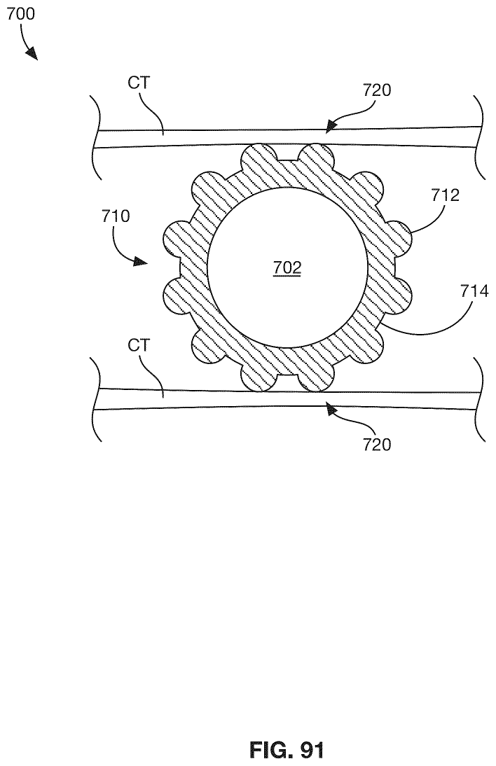

Referring now to FIG. 90, a cross-sectional view of a portion of a cover 710 on a portion of an implantable device 700 is shown, which can be any of the valve repair devices shown and described herein or any other known valve repair device. The device 700 is shown arranged between two chordae tendineae CT. In FIG. 90, the device 700 can travel in the direction indicated by double arrow 701, with the chordae tendinea extending into or out of the page. FIG. 91 is a cross-sectional views taken along the plane indicated by lines 91-91 in FIG. 90. In FIG. 91, the device 700 is shown arranged between two chordae tendineae CT such that the direction of travel 601 of the device 700 would be in or out of the plane of the page. As such, the device 700 would be moved in a direction 701 that is orthogonal to the length of the chordae tendineae CT.

The device 700 includes a device body 702 that is covered by a cover 710. The cover 710 includes ridge portions 712 having a major outer diameter spaced apart by valley portions 714 having a minor outer diameter. The chordae tendineae CT are in contact with the ridge portion 712 at a contact area 720. As the device 700 moves against the chordae tendineae CT, the contact area 720 tends to maintain a substantially constant size because the ridge portions 712 are oriented orthogonal to the length of the chordae tendineae CT and pressure applied by the ridge portions 712 against the chordae tendineae CT can cause the chordae tendineae CT to come into contact with adjacent ridge portions 712 but not to move into the valley portions 714 or to change the size of the contact area 720 with each ridge portion 712. Further movement can cause the chordae tendineae CT to slide along the ridge portions 712 such that the force required to move the device 700 remains steady. In this way, friction between the chordae tendineae CT and the device 700 is reduced.

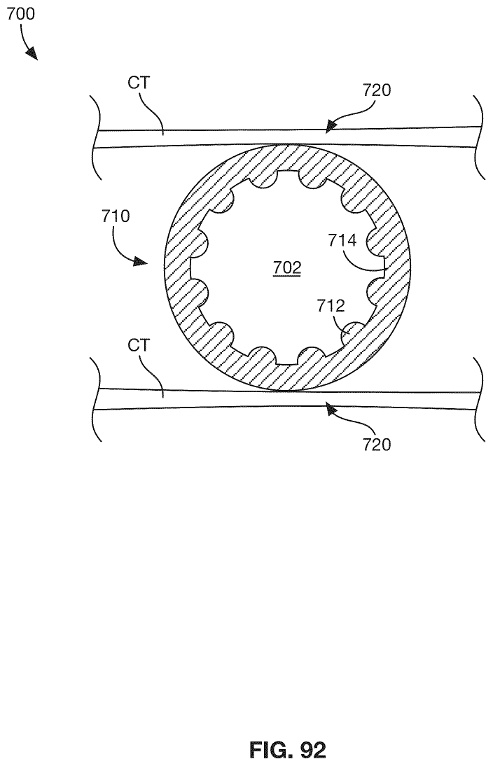

The cover 710 for the device 700 can also be turned inside-out so that the ridge portions 712 and valley portions 714 are facing inwards and the backside of the cover 710 is facing outwards, as is shown in FIG. 92. In this configuration, the thickness of the material of the cover 710 remains the same but a smooth exterior is presented. This allows for the same tissue ingrowth capacity and can change the friction performance of the cover 710. In particular, turning the cover 710 inside-out reduces friction significantly as the ridge and valley portions 612, 614 are no longer exposed to the chordae tendineae CT.

In some implementations, the cover material 610 illustrated by FIGS. 87-89 can be used for the first cover portion 540 in the examples illustrated by FIGS. 72-86 and the cover material 710 illustrated by FIGS. 90-92 can be used for the second cover portion 550 in the examples illustrated by FIGS. 72-86. In some implementations, the cover materials 610 and 710 are the same materials that are oriented differently. For example, the cover material 710 can be the same as the cover material 610 but rotated 90 degrees.

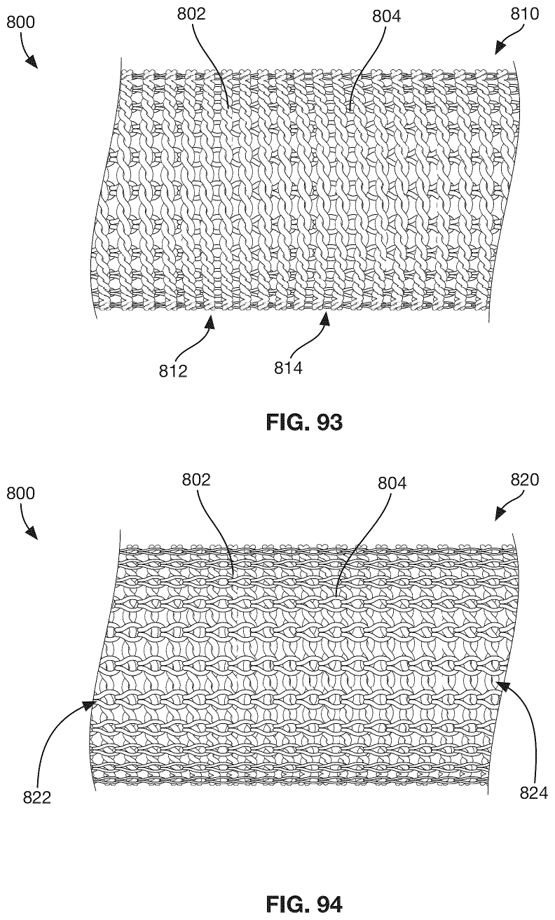

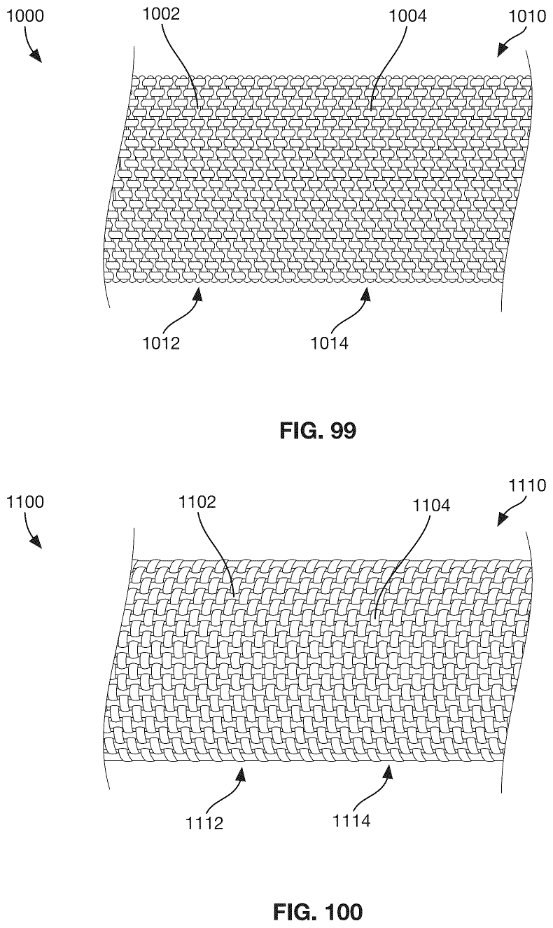

Referring now to FIGS. 93-100, example materials and data illustrating the friction of the various materials are shown. These materials, such as the knitted materials shown in FIGS. 93-96 and the woven materials shown in FIGS. 99-100 can be used to cover any of the devices disclosed herein. Similar surface features shown for the knitted and woven materials could also be formed by molding or otherwise forming grooves in the exterior surface of an outer layer or coating (e.g., the coatings discussed above with respect to the device 400) of an example cover for an implantable device/implant.

Referring now to FIGS. 93-96, example knitted covers for an implantable device/implant are shown. Knitted materials can be formed from one or more yarns or strands of material that are stitched together by forming a series of loops. The yarn or strand of material follows a meandering path-a course-through the material as the loops for the stitches are formed. A sequence of stitches that suspend from each other is called a wale. For weft knitted materials the courses run orthogonal to the wales such that courses are made by adding stitches to each wale until the knitted material reaches a desired size.

Referring now to FIGS. 93-94, a first knitted cover 800 is shown with a first side 810 oriented outwards in FIG. 93 and a second side 820 oriented outwards in FIG. 94. That is, the cover 800 of FIG. 93 is turned inside-out to become the cover 800 of FIG. 94. Courses 802 of the first knitted cover 800 are oriented circumferentially so that the courses 802 wrap around the tube shape of the first knitted cover 800. Wales 804 of the first knitted cover 800 are oriented longitudinally so that the wales 804 extend along the length of the tube shape of the first knitted cover 800. Circumferentially oriented ridge portions 812 spaced apart by circumferentially oriented valley portions 814 are formed on the first side 810 of the first knitted cover 800 by the courses 802. Longitudinally oriented ridge portions 822 spaced apart by longitudinally oriented valley portions 824 are formed on the second side 820 of the first knitted cover 800 by the wales 804. The ridge portions 822 formed by the wales 804 protrude more than the ridge portions 812 formed by the courses 802 so that the valley portions 824 of the second side 820 are deeper than the valley portions 814 of the first side 810.

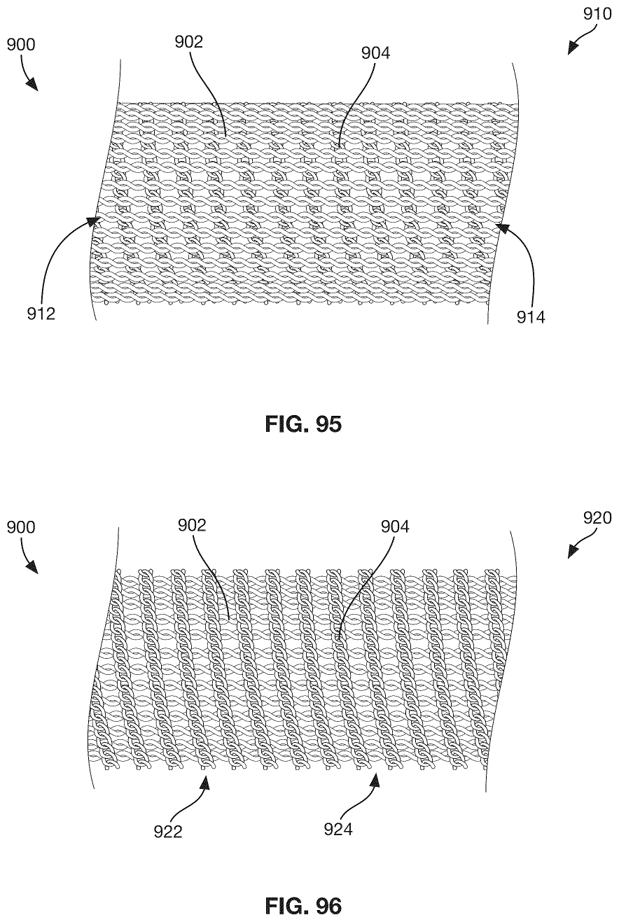

Referring now to FIGS. 95-96, a second knitted cover 900 is shown with a first side 910 oriented outwards in FIG. 95 and a second side 920 oriented outwards in FIG. 96. That is, the cover 900 of FIG. 95 is turned inside-out to become the cover 900 of FIG. 96. The second knitted cover 900 is knitted in a similar manner to the first knitted cover 800 but the knitting pattern is rotated 90 degrees so that the surface features of the second knitted cover 900 are orthogonal to the surface features of the first knitted cover 800. For example, courses 902 of the second knitted cover 900 are oriented longitudinally so that the courses 902 extend along the length of the tube shape of the second knitted cover 900. Wales 904 of the second knitted cover 900 are oriented circumferentially so that the wales 904 wrap around the tube shape of the second knitted cover 900. Longitudinally oriented ridge portions 912 spaced apart by longitudinally oriented valley portions 914 are formed on the first side 910 of the second knitted cover 900 by the courses 902. Circumferentially oriented ridge portions 922 spaced apart by circumferentially oriented valley portions 924 are formed on the second side 920 of the second knitted cover 900 by the wales 904. The ridge portions 922 formed by the wales 904 protrude more than the ridge portions 912 formed by the courses 902 so that the valley portions 924 of the second side 920 are deeper than the valley portions 914 of the first side 910.

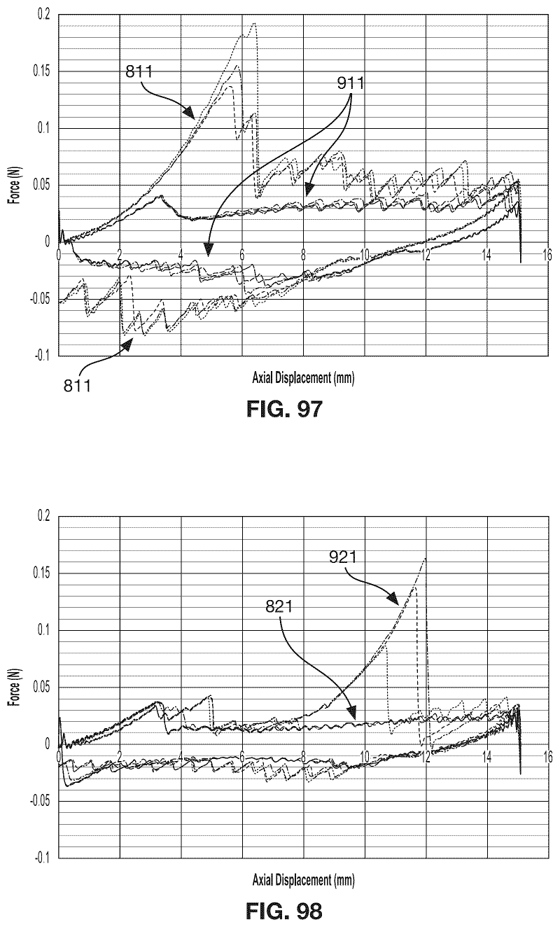

FIGS. 97-98 are graphs of data that illustrates the different forces required to displace probes with different covers along tissue samples. In these examples a probe covered by the first and second knitted covers 800, 900 having the first sides 810, 910 and the second sides 820, 920 facing outward to engage tissue samples were tested.

Data comparing the first sides 810, 910 is shown in FIG. 95. The force data for the first sides 810, 910 are plotted as data series 811, 911, respectively. A comparison of the data of the data series 811, 911 shows that the first side 910 of the second knitted cover 900 required less force to move while engaging the native tissue and therefore has a lower coefficient of friction than the first side 810 of the first knitted cover 800.

As can be seen in FIG. 97, the force data 811 ramps up to a first peak and then again to successive smaller peaks while the force data 911 maintains a lower overall value. The large peak in the force data 811 can be attributed to the native tissue engaging and getting caught in a valley portion 814 against one of the ridge portions 812. The caught ridge portion 812 can then move with the native tissue and collide against successive ridge portions 812 causing the material of the first knitted cover 800 to bunch up, forming a larger obstacle for the native tissue to overcome. At some point the tension in the native tissue causes the native tissue to stretch around the bunched-up ridge portions 812, thereby quickly relieving the force experienced by the probe that shows as a sharp decline in the force data 811 following the large peak. Similar sticking and slipping may be seen during implantation of a device covered with a cloth material. Smaller peaks later in the data 811 can be caused again by the native tissue becoming caught on one or more of the ridge portions 812. The force data 911 recorded for the first side 910 of the second knitted cover 900 does not show peaks as large as the force data 811. When comparing data sets, the friction forces of particular cover materials can be compared based on the steady state value—that is, the smaller peaks and valleys in the data and not the large peaks that may be caused by bunching.

The data of FIG. 98 illustrates a similar scenario to that of FIG. 97. That is, a comparison between force data 821 from the first knitted cover 800 and force data 921 from the second knitted over 900 shows that less force is required to move the surface having longitudinally oriented surface features—i.e., the ridge portions 822 of the first knitted cover 800 than the surface having circumferentially oriented features—i.e., the ridge portions 922 of the second knitted cover 900. The decreased friction of the longitudinally oriented ridge portions 822, 912 results from a decreased contact area between the ridge portions 822, 912 and the native tissue and because the native tissue cannot be caught in valley portions 824, 914 oriented askew from or orthogonal to the native tissue.

Comparing the two data sets for surfaces having longitudinally oriented features, i.e., the second side 820 of the first knitted cover 800 and the first side 910 of the second knitted cover 900, further reveals that the ridge portions 822 formed by the wales 804 of the first knitted cover 800 provide a lower friction surface than the ridge portions 912 formed by the courses 902 of the second knitted cover 900. As can be seen in FIG. 96, the wales 904 (and similarly, the wales 804 of the first knitted cover 800) form larger ridge portions 822, 922 and, consequently, deeper valley portions 824, 924 than the ridge portions 812, 912 and valley portions 814, 914 formed by the courses 802, 902). There are also fewer wales 804 than courses 902, resulting in a smaller total contact patch between the native tissue and the ridge portions 822 than between the native tissue and the ridge portions 912.

Referring now to FIGS. 99-100, first and second woven covers 1000, 1100 are shown with a first side 1010, 1110 oriented outwards. Second sides of the first and second woven covers 1000, 1100 are not shown and would be similar in appearance to FIGS. 99-100, respectively. Woven materials can be formed by weaving one or more weft yarns or strands of material in and out of a plurality of warp yarns or strands. The weft strands follow a somewhat straight path that is orthogonal to the warp strands. The relatively straight path of the weft and warp strands, in contrast to the meandering path of the strands in a knitted material, provides less elasticity than knitted materials. Woven materials can be stronger than knitted materials, and also tend to include smaller openings in the material as the warp and weft strands are more closely packed together than the courses and wales of a knitted material. The first and second woven covers 1000, 1100 can be formed of any suitable material, such as, for example, polyethylene terephthalate (PET) or the like.

Referring now to FIG. 99, a first woven cover 1000 is shown with the first side 1010 oriented outwards. A second side of the first woven cover 1000 is not shown but would be similar in appearance to the first side 1010. Warp strands 1002 of the first woven cover 1000 are oriented circumferentially so that the warp strands 1002 wrap around the tube shape of the first woven cover 1000. Weft strands 1004 of the first woven cover 1000 are oriented longitudinally so that the weft strands 1004 extend along the length of the tube shape of the first woven cover 1000. Circumferentially oriented ridge portions 1012 spaced apart by circumferentially oriented valley portions 1014 are formed on the first side 1010 of the first knitted cover 1000 by the warp strands 1002. We note that the ridge and valley portions 1012, 1014 are smaller in height and width than, for example, the ridge and valley portions 812, 814 of the first knitted cover 800.

Referring now to FIG. 100, a second woven cover 1100 is shown with the first side 1110 oriented outwards. A second side of the second woven cover 1100 is not shown but would be similar in appearance to the first side 1110. Warp strands 1102 of the second woven cover 1100 are oriented longitudinally so that the warp strands 1102 extend along the length of the tube shape of the second woven cover 1100. Weft strands 1104 of the second woven cover 1100 are oriented circumferentially so that the weft strands 1104 wrap around the tube shape of the second woven cover 1100. Longitudinally oriented ridge portions 1112 spaced apart by longitudinally oriented valley portions 1114 are formed on the first side 1110 of the second knitted cover 1100 by the warp strands 1102. We note that the ridge and valley portions 1112, 1114 are smaller in height and width than, for example, the ridge and valley portions 812, 814 of the first knitted cover 800.

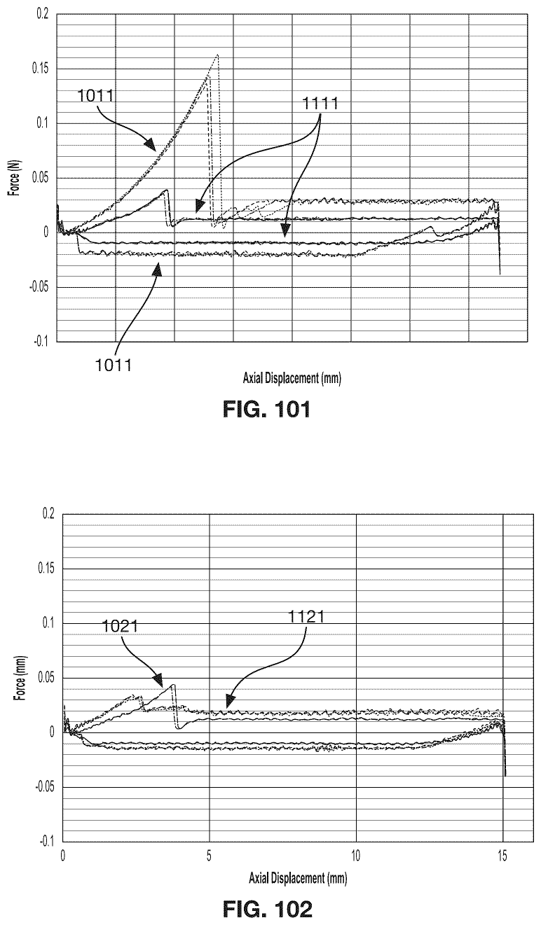

Referring now to FIGS. 101-102, data illustrating the force required to displace a probe covered by the first and second woven covers 1000, 1100 having the first sides 1010, 1110 and the second sides (not shown) facing outward to engage tissue samples. Data comparing the first sides 1010, 1110 is shown in FIG. 101. The force data for the first sides 1010, 1110 are plotted as data series 1011, 1111, respectively. A comparison of the data of the data series 1011, 1111 shows that the first side 1110 of the second knitted cover 1100 required less force to move while engaging the native tissue and therefore has a lower coefficient of friction than the first side 1010 of the first knitted cover 1000. Data for the second sides (not shown) of the first and second woven covers 1000, 1100 are shown in data series 1021, 1121 of FIG. 102.

Similar to the first and second knitted covers 800, 900, the data from testing the first and second woven covers 1000, 1100 show that longitudinally oriented surface features reduce friction with the native tissue. Also, the data shows that the native tissues tend to become caught on circumferentially oriented surface features, such as the warp strands 1002, leading to a spike in the force required to move past the native tissue.

When comparing the knitted covers 800, 900 to the woven covers 1000, 1100 one also can see that the orientation of the material forming the knitted covers 800, 900 has a more significant impact on the friction between the cover material and the native tissue. In other words, the woven covers 1000, 1100 are less sensitive to changes in orientation. This is likely because of the smaller overall surface features of the woven covers 1000, 1100. Thus, an advantage of a woven material such as the woven covers 1000, 1100 is that the orientation of the fabric does not need to be closely controlled during manufacturing. Knitted materials, such as those used to make the knitted covers 800, 900 are more elastic than woven materials because of the meandering path of the yarn or strand used to form the knitted material. Thus, an advantage of knitted materials such as the materials used to make the knitted covers 800, 900 is that the resulting cover more easily conforms to different shapes and flexes and stretches as the underlying device moves and changes size, shape, or position.

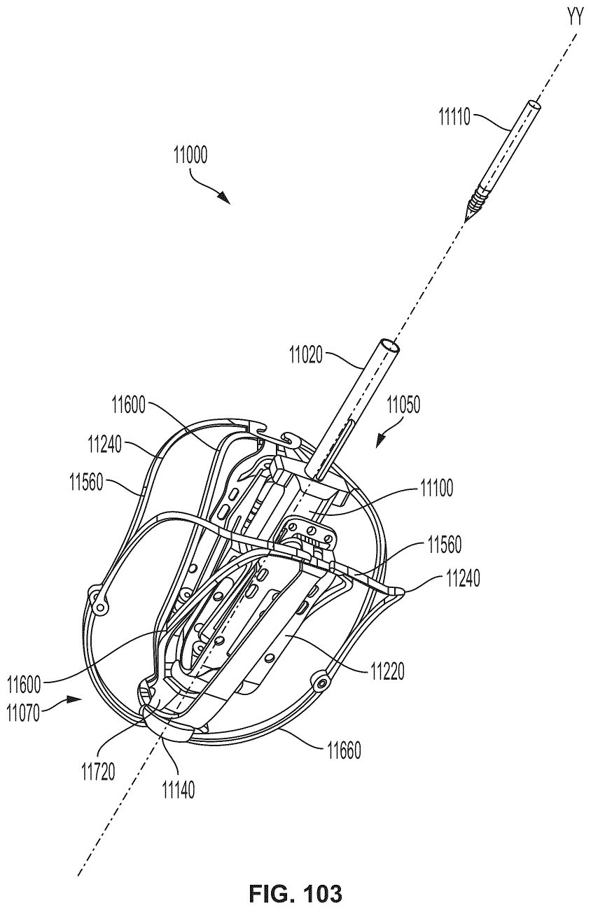

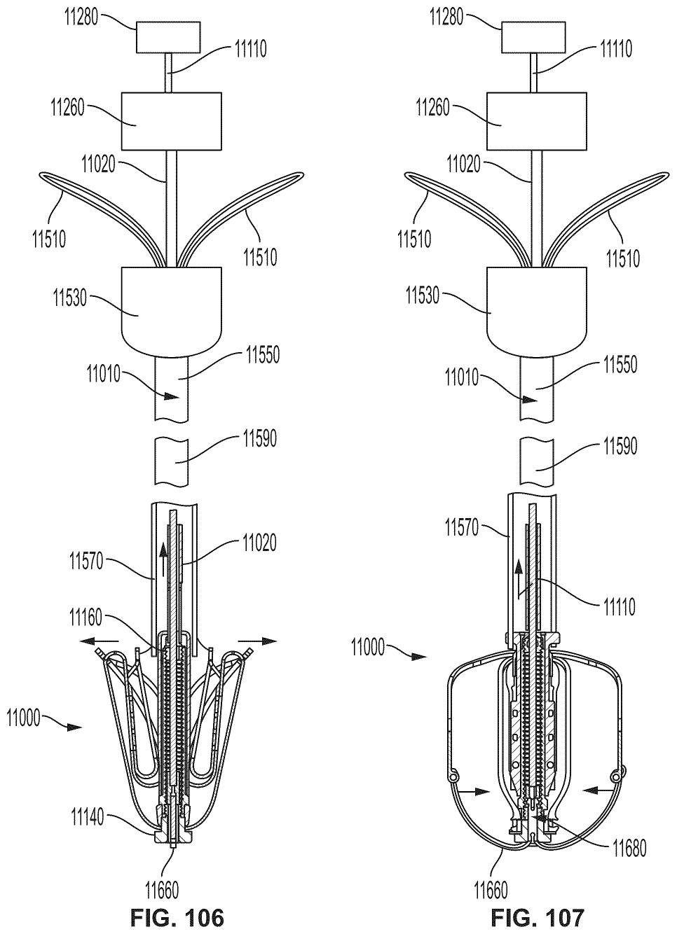

FIGS. 103-107 illustrate an example of a valve repair system for repairing a native valve of a patient. The valve repair system can include a delivery device 11010 (FIGS. 106-107) and an implantable valve repair device 11000. Referring to FIGS. 103-105, the implantable device 11000 includes a proximal or attachment portion 11050, paddle frames 11240, and a distal portion 11070. The proximal portion 11050, the distal portion 11070, and the paddle frames 11240 can be configured in a variety of ways.

In the example illustrated in FIG. 103, the paddle frames 11240 can be symmetric along longitudinal axis YY. However, in some implementations, the paddle frames 11240 are not symmetric about the axis YY Moreover, referring to FIG. 103, the paddle frames 11240 can include outer frame portions 11560 and inner frame portions 11600.

In the illustrated implementation, the outer frame portions 11560 are flexibly attached to outer end portions of a w-shaped connector 11660 (e.g., shaped metal component, shaped plastic component, tether, wire, strut, line, cord, suture, etc.). Between the connector 11660 and the proximal portion 11050, the outer frame portions 11560 form a curved shape. For example, in the illustrated example, the shape of the outer frame portions 11560 resemble an apple shape in which the outer frame portions 11560 are wider toward the proximal portion 11050 and narrower toward the distal portion 11070. In some implementations, however, the outer frame portions 11560 can be otherwise shaped.

The inner frame portions 11600 extend from the proximal portion 11050 toward the distal portion 11070. The inner frame portions 11600 then extend inward to form retaining portions 11720 that are attached to the actuation cap 11140. The retaining portions 11720 and the actuation cap 11140 can be configured to attach in any suitable manner.

In some implementations, the inner frame portions 11600 are rigid frame portions, while the outer frame portions 11560 are flexible frame portions. The proximal end of the outer frame portions 11560 connect to the proximal end of the inner frame portions 11600, as illustrated in FIG. 103.

The width adjustment element 11110 is configured to move the outer frame portions 11560 from the expanded position to the narrowed position by pulling an inner end 11680 of the connector 11660 (FIGS. 105 and 107) in a proximal direction relative to the actuation cap 11140. In some implementations, portions of the connector 11660 move through the actuation cap 11140 and into a receiver 11120 (e.g., an internally threaded element, notched receiving portion, column, lumen, tube, shaft, post, etc.) when outer frame portions 11560 are moved to the narrowed position. The actuation element 11020 can be configured to engage the receiver 11120 and/or cap 11140 to move the inner paddle frame portions 11600 to open and close the paddles.

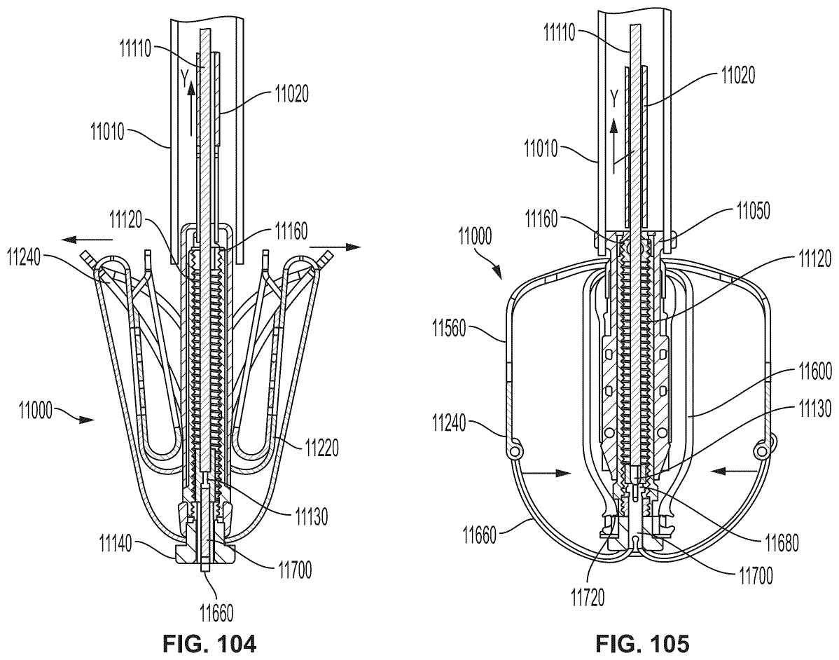

As shown in FIGS. 104 and 105, the connector 11660 has an inner end 11680 that engages with the width adjustment element 11110 such that a user can move the inner end 11680 relatively inside the receiver 11120 to move the outer frame portions 11560 between a narrowed position and an expanded position. In the illustrated example, the inner end 11680 of the connector 11660 includes a post 11700 that attaches to the outer frame portions 11560 and a coupler 11130 that extends from the post 11700. The coupler 11130 is configured to attach and detach from both the width adjustment element 11110 and the receiver 11120. When the coupler 11130 is attached to the width adjustment element 11110, the coupler 11130 is released from the receiver 11120. When the coupler 11130 is detached from the width adjustment element 11110, the coupler is secured to the receiver 11120. The inner end 11680 of the connector 11660 can, however, be configured in a variety of ways. Any configuration that can suitably attach the connector 11660 to the coupler to allow the width adjustment element 11110 to move the outer frame portions 11560 between the narrowed position and the expanded position can be used.

The width adjustment element 11110 allows a user to expand or contract the outer frame portions 11560 of the implantable device 11000. In the example illustrated in FIGS. 104 and 105, the width adjustment element 11110 includes an externally threaded end that is threaded into the coupler 11130. The width adjustment element 11110 moves the coupler in the receiver 11120 to adjust the width of the outer frame portions 11560. When the width adjustment element 11110 is unscrewed from the coupler 11130, the coupler 11130 engages the inner surface of the receiver 11120 to set the width of the outer frame portions 11560.

In some implementations, the receiver 11120 can be integrally formed with the cap 11140. Moving the cap 11140 relative to a coaptation element that is connected to the attachment portion 11050 opens and closes the paddles. In the illustrated example, the receiver 11120 slides inside the coaptation element. When the coupler 11130 is detached from the width adjustment element 11110, the width of the outer frame portions 11560 is fixed while the actuation element 11020 moves the receiver 11120 and cap 11140 relative to the coaptation element. Movement of the cap 11140 can open and close the device in the same manner as the other implementations disclosed above.

In the illustrated example, a driver head 11160 is disposed at a proximal end of the actuation element 11020. The driver head 11160 releasably couples the opening/closing actuation element 11020 to the receiver 11120. In the illustrated example, the width adjustment element 11110 extends through the receiver 11120. The receiver 11120 is axially advanced in the direction opposite to direction Y to move the cap 11140. Movement of the cap 11140 relative to the attachment portion 11050 is effective to open and close the paddles, as indicated by the arrows in FIG. 104. That is movement of the cap 11140 in the direction Y closes the device and movement of the cap in the direction opposite to direction Y opens the device.

Also illustrated in FIGS. 104 and 105, the width adjustment element 11110 extends through the actuation element 11020, the driver head 11160, and the receiver 11120 to engage the coupler 11130 attached to the inner end 11680 of the connectors 11660. The movement of the outer frame portions 11560 to the narrowed position can allow the device or implant 11000 to maneuver more easily into position for implantation in the heart by reducing the contact and/or friction between the native structures of the heart—e.g., chordae—and the device 11000. The movement of the outer frame portions 11560 to the expanded position provides the anchor portion of the device or implant 11000 with a larger surface area to engage and capture leaflet(s) of a native heart valve.

Referring to FIGS. 106 and 107, an implementation of an implant catheter assembly 11010 in which clasp actuation lines 11510 extend through a handle 11530, the actuation element 11020 is coupled to a paddle actuation control 11260, and the width adjustment element 11110 is coupled to a paddle width control 11280 is shown. A proximal end portion 11550 of the shaft or catheter of the catheter assembly 11010 can be coupled to the handle 11530, and a distal end portion 11570 of the shaft or catheter can be coupled to the implantable device 11000. The actuation element 11020 can extend distally from the paddle actuation control 11260, through the handle 11530, through the delivery shaft or catheter of the delivery device 11010, and through the proximal end of the device 11000, where it couples with the driver head 11160. The actuation element 11020 can be axially movable relative to the outer shaft of the catheter assembly 11010 and the handle 11530 to open and close the device.

The width adjustment element 11110 can extend distally from the paddle width control 11280, through the paddle actuation control 11260 and through the actuation element 11020 (and, consequently, through the handle 11530, the outer shaft of the implant catheter assembly 11010, and through the device 11000), where it couples with the movable coupler 11130. The width adjustment element 11110 can be axially movable relative to the actuation element 11020, the outer shaft of the catheter assembly 11010, and the handle 11530. The clasp actuation lines 11510 can extend through and be axially movable relative to the handle 11530 and the outer shaft of the catheter assembly 11010. The clasp actuation lines 11510 can also be axially movable relative to the actuation element 11020.

Referring to FIGS. 106 and 107, the width adjustment element 11110 can be releasably coupled to the coupler 11130 of the device 11000. Advancing and retracting the width adjustment element 11110 with the control 11280 widens and narrows the paddles. Advancing and retracting the actuation element 11020 with the control 11260 opens and closes the paddles of the device.

In the examples of FIGS. 106 and 107, the catheter or shaft of the implant catheter assembly 11010 is an elongate shaft extending axially between the proximal end portion 11550, which is coupled to the handle 11530, and the distal end portion 11570, which is coupled to the device 11000. The outer shaft of the catheter assembly 11010 can also include an intermediate portion 11590 disposed between the proximal and distal end portions 11550, 11570.

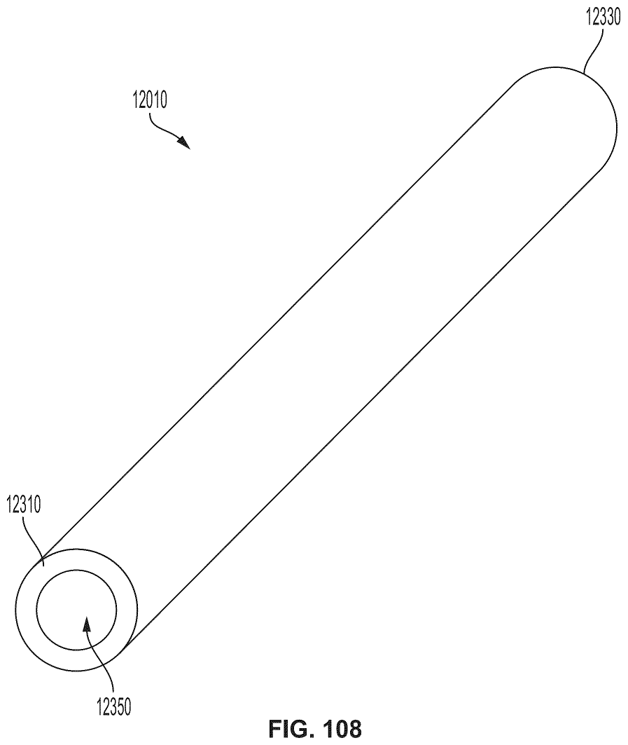

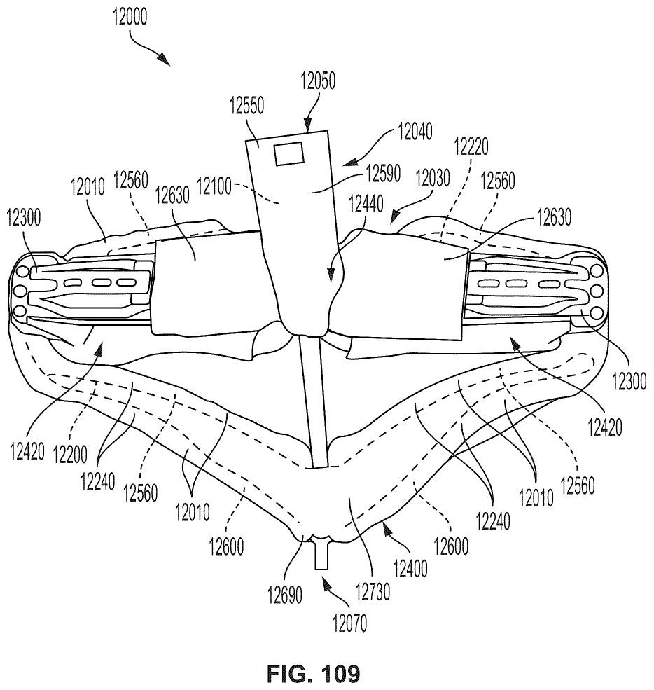

Referring now to FIGS. 108-119, an example sleeve 12010 (FIG. 108) and cover assembly 12030 (FIGS. 109-117) for attaching to an example implantable device 12000 is shown. The sleeve 12010 and/or cover assembly 12030 can be used with any suitable type of implantable device, such as, for example, the device or implant 100 shown in FIGS. 8-15; the implantable device 11000 shown in FIGS. 103-108; the devices/implants described in detail in PCT patent application publication Nos. WO2018/195215, WO2020/076898, and WO 2019/139904 (which are incorporated herein by reference in their entirety for all purposes), or any combinations thereof. The implantable device 12000 can include any other features for an implantable device or implant discussed in the present application or the applications cited above, and the device 12000 can be positioned to engage valve tissue as part of any suitable valve repair system (e.g., any valve repair system disclosed in the present application or the applications cited above).

In the illustrated implementation, referring to FIGS. 109-112 and 116-118, the implantable device 12000 includes a proximal or attachment portion 12050, a coaptation portion 12040 having a coaptation element 12100, an anchor portion 12060, and a distal portion 12070. The proximal portion 12050, the coaptation portion 12040, the anchor portion 12060, and the distal portion 12070 can be configured in a variety of ways, such as, for example, any of the ways described in the present application or the applications cited above.

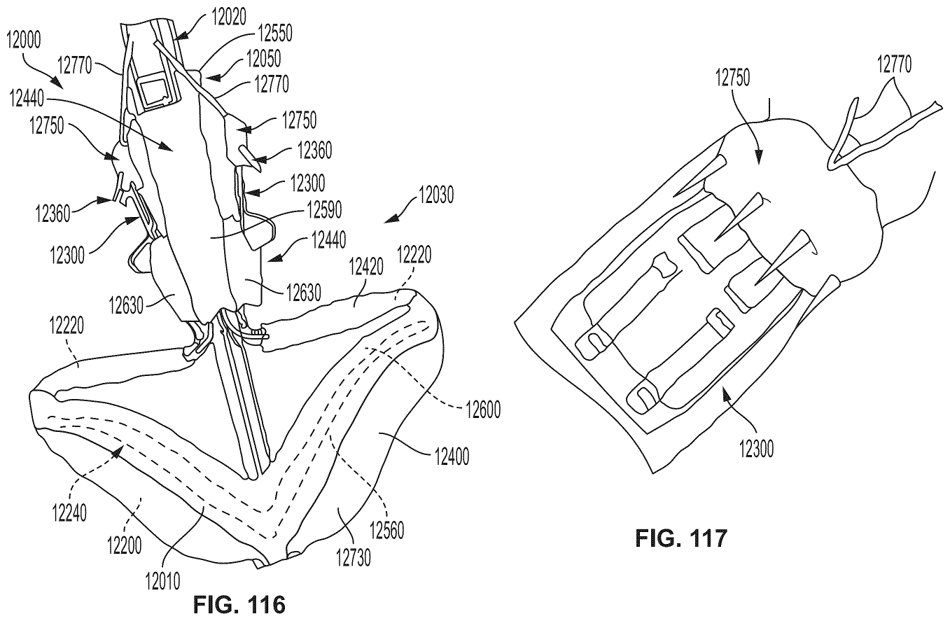

The device or implant 12000 is deployed from a delivery system 12020 (FIG. 116) or other means for delivery. The delivery system 12020 can comprise one or more of a catheter, a sheath, a guide catheter/sheath, a delivery catheter/sheath, a steerable catheter, an implant catheter, a tube, a channel, a pathway, actuation elements, combinations of these, etc. The delivery system 12020 can be configured in a variety of ways, such as, for example, any of the ways described in the present application or the applications cited above.

In some implementations, the coaptation portion 12040 of the device or implant 12000 includes a coaptation element 12100 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element (e.g., actuation wire, actuation shaft, actuation tube, etc.) of the delivery system 12020. The coaptation element 12100 can be configured in a variety of ways, such as, for example, any of the ways described in the present application or the applications cited above.

The anchor portion 12060 can include one or more anchors 12080 that are actuatable between open and closed positions. The anchors 12080 can take a wide variety of forms, such as, for example, any of the ways described in the present application or the applications cited above. In the illustrated implementation, each of the anchors 12080 have an inner paddle 12220, an outer paddle 12200, and gripping element or clasp 12300. The anchors 12080 can also have paddle frames 12240. The paddle frames 12240 can be configured in a variety of ways, such as, for example, the configuration of the paddle frames 11240 shown in FIGS. 103-107, or any of the other ways described in the present application or the applications cited above. In the illustrated implementation, the paddle frames 12240 include outer frame portions 12560 and inner frame portions 12600.

Actuation of an actuation element (see FIG. 106) opens and closes the anchor portion 12060 of the device 12000 to grasp the native valve leaflets during implantation. The actuation element can take a wide variety of different forms (e.g., a wire, rod, shaft, tube, screw, suture, line, strip, combination of these, etc.), be made of a variety of different materials, and have a variety of configurations. In certain implementations, the actuation element can take the form of the actuation element 11020 shown in FIGS. 103-107.

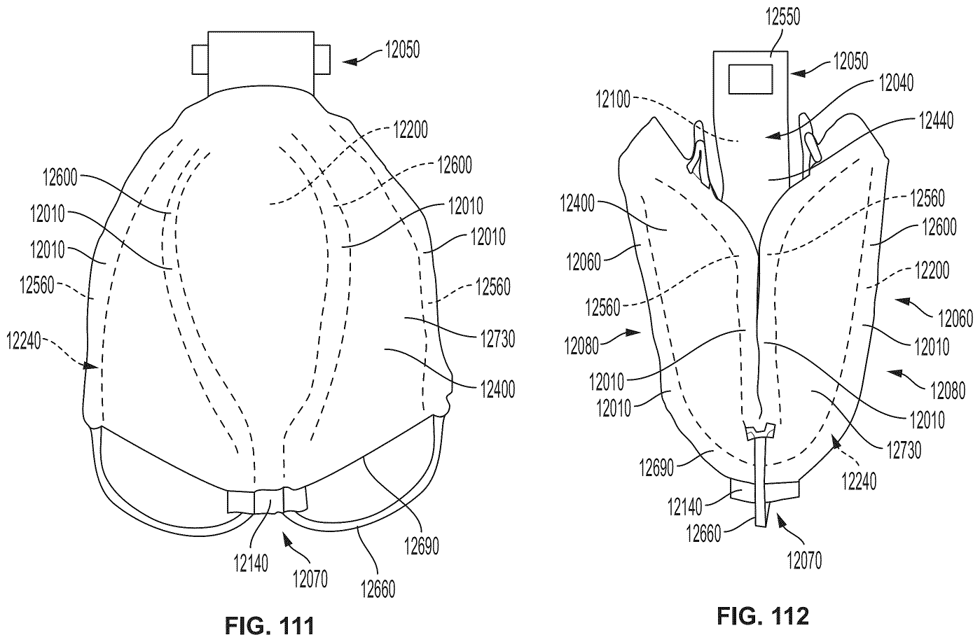

Referring to FIG. 108, the sleeve 12010 can be a cylindrical tube having a first end 12310, a second end 12330, and a lumen 12350 extending therebetween. In certain implementations, one or more sleeves 12010 are disposed on portion(s) of the paddle frame 12240. For example, referring to FIG. 111, a sleeve 12010 can be disposed over each of the struts or elongated wire-like portions of both the inner frame portions 12600 and outer frame portions 12560 of the paddle frames 12240 for each of the anchors 12080 (see also FIG. 103 where the inner frame portions 11600 and outer frame portions 11560 are not covered—the paddle frames 12240 can be the same or substantially the same as the paddle frames 11240). As a result, there are four sleeves on each side of the illustrated device (i.e., one sleeve over each of the two struts or elongated wire-like portions of the inner frame portion 12600 and one sleeve over each of the two struts or elongated wire-like portions of the outer frame portion 12560). Since there is a paddle frame 12240 on each side of the device, there are a total of eight sleeves in the illustrated example.

A sleeve or sleeves 12010 can, however, be placed on any portion or portions of a paddle frame 12240. Referring to FIG. 108, while the illustrated implementation shows the sleeve 12010 being a cylindrical tube, it should be understood that the sleeve 12010 can take any suitable form that at least partially covers or surrounds a member of the paddle frame 12240. The sleeve 12010 can be made of any suitable material, such as, for example, polyethylene. In certain implementations, the sleeve 12010 can be made from braided polyethylene terephthalate (PET) with a spin finish on the yarn. In some implementations, the sleeve 12010 is made of a material that promotes tissue ingrowth and/or provides the reduced friction benefits of any of the examples disclosed herein. The sleeve 12010 can be stretchable such that compression of the sleeve 12010 causes a diameter of the lumen to become larger, and such that extension of the sleeve 12010 causes the diameter of the lumen to become smaller.

In certain implementations, the sleeves 12010 can allow for a cover (e.g., portions of the cover assembly 12030 described in the present application) to be easily attached to the device 12000 and/or that provides edges of the device with low friction to easily slide past native structures of the heart, such as chordae tendinea. The cover can be attached to the sleeve 12010 by one or more connectors (e.g., stitches, adhesive, mechanical fasteners, ultrasonic welds, etc.). The sleeve 12010 can prevent or inhibit protrusions extending from the one or more connectors from extending past an external surface of the device. For example, stitches can extend into the sleeve 12010, rather than extending around a portion of the paddle frames 12240. The sleeves 12010 can be made of a material that is strong enough to receive and hold connectors, such as stitches, for connecting a cover to the sleeves 12010.

In some implementations, the sleeve 12010 can be lubricious so as to allow the device 12000 to maneuver more easily into position for implantation in the heart by reducing the friction between the native structures of the heart—e.g., chordae—and edges of the device 11000 that are covered by the sleeves. For example, in implementations in which the sleeve 12010 is made of braided PET with a spin finish, the spin finish acts as a lubricant. In some implementations, the sleeve 12010 can be coated with a lubricious substance. In certain implementations, the sleeve 12010 can be made of a material that is inherently lubricious. The sleeve 12010 can have a lower coefficient of friction than other components of the device 12000. For example, the sleeve 12010 can have a lower coefficient of friction than the paddle frames, cover(s) of the cover assembly 12030, and/or any other component of the device 12000. Any of the friction reducing features of any of the implementations disclosed herein can be applied to the sleeve.

Referring to FIGS. 109-117, the device 12010 can include a cover assembly 12030 having one or more covers for attaching to various components of the device. The covers of the cover assembly 12030 can be configured to act as a barrier that prevents or inhibits the movement of blood through the native valve, assist in coapting leaflets by providing further engagement with the leaflets, and/or promote tissue ingrowth. Each cover can include a sheet, material, fabric, layer, and/or membrane that is attached to one or more components of the device 12000 by one or more connectors (e.g., stitches, adhesive, mechanical fasteners, ultrasonic welds, etc.). The sheet, material, fabric, layer, and/or membrane can be made of any suitable material, such as, for example, polyethylene. For example, the sheet, material, fabric, layer, and/or membrane can be made of a polyethylene cloth of fine mesh, a knitted PET, a woven PET, or any other suitable type of polyethylene material. In some implementations, the sheet, material, fabric, layer, and/or membrane can be made of a flexible material, a porous or non-porous material, and/or a material that is impermeable to (or inhibits or impedes) blood flow. In some implementations, the sheet, material, fabric, layer, and/or membrane is made from or comprises a biocompatible material, such as a woven biocompatible fabric that is configured to promote tissue ingrowth.

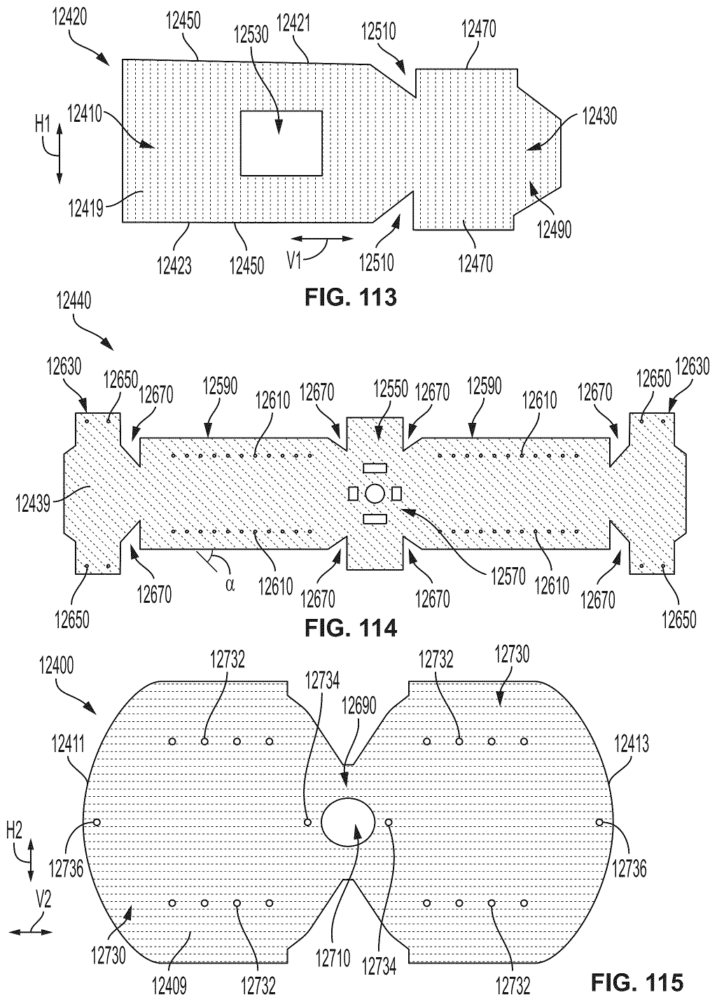

In the illustrated implementation, the cover assembly 12030 includes a first cover 12400 for attaching to the paddle frames 12240, second covers 12420 for attaching to the inner paddles 12220, and a third cover 12440 for attaching to the coaptation element 12100 and the clasps 12300. Referring to FIGS. 113-115, these portions of the cover assembly 12030 are shown cut from flat sheets of material. Each of the covers 12400, 12420, 12440 include different shaped segments or portions to attach to different portions of the device 12000. The covers 12400, 12420, 12440 can be shaped to smooth transitions between portions of the device 12000 and to reduce catch points and provide a smoother exterior to the device.

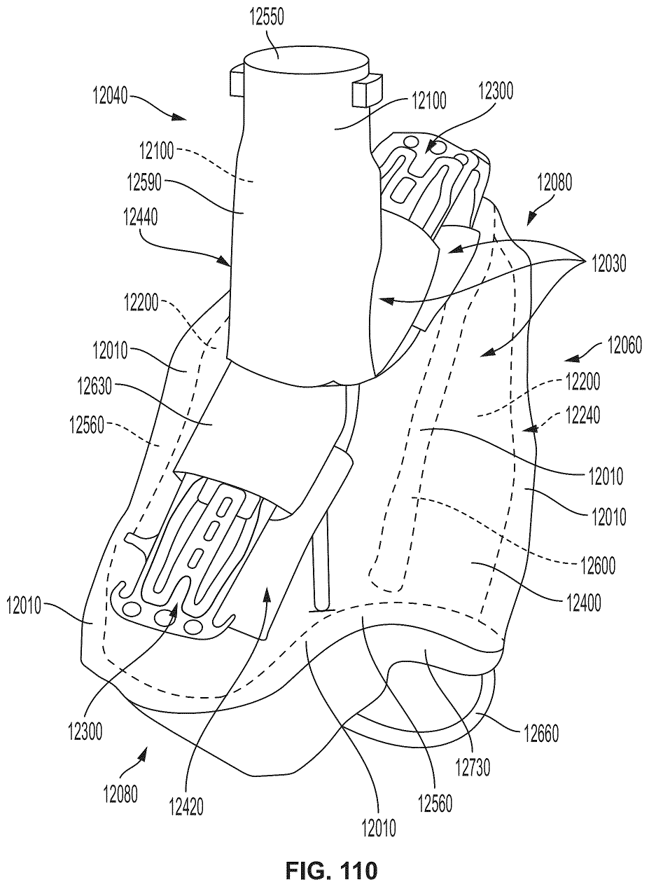

Referring to FIG. 113, the second cover 12420 is configured to be disposed on the inner paddle 11220 (see FIGS. 109 and 110 where the inner paddle 12220 is covered and see FIG. 103 where the inner paddle 11220 is not covered). The second cover 12420 can have a first portion 12410 and a second portion 12430. The first portion 12410 can be configured for being disposed on the inner paddle 12220 proximate a central portion of the device 12000 (e.g., proximate the coaptation element 12100—see also the uncovered coaptation element 11100 in FIG. 103), and the second portion 12430 can be configured for being disposed on a portion of the inner paddle 12220 that extends furthest away from the central portion of the device 12000 when the anchors 12080 are in the open position. In certain implementations, the second cover 12420 is attached to the inner paddle 12220 by placing the cover 12420 on an upper or proximate surface of the inner paddle 12220, wrapping side edges 12450, 12470 of both the first and second portions 12410, 12430 around the inner paddle, and attaching the cover 12420 to the inner paddle by one or more connectors. In the illustrated implementation, the cover 12420 includes an end portion 12490 that is configured to attach to another cover of the cover assembly 12030 (e.g., the cover 12400 that is disposed over the paddle frames and the outer paddle) or another component of the device 12000 (e.g., the paddle frame 12240) to further secure the cover 12420 to the device 12000.

The cover 12420 can include one or more cutout portions 12510 that allows for the cover 12420 to be wrapped around the inner paddle 12220 in a smooth manner. In some implementations, the second cover 12420 also covers a fixed arm (not shown) of the clasp 12300 that is fixed to the inner paddle 12220. In certain implementations, the second cover 12420 includes a window or opening 12530 that allows an indicator (not shown) to be visible to a user during implantation of the device 12000. For example, the device 12000 can have an indicator, such as any of the indicators shown in U.S. Provisional Patent Application Ser. No. 63/225,387, filed on Jul. 23, 2021, which is incorporated herein by reference in its entirety, and the window 12530 allows the indicator to be visible to a user.

The second cover 12420 can have threads 12419 extending in a horizontal direction H1 (e.g., by laser cutting the cover 12420) from a first side edge 12421 to a second side edge 12423 of the cover 12420. The horizontal direction of the threads 12419 can allow for connectors to be easily attached to the cover 12420, as well as allow for stretching of the cover in the vertical direction V1. While the second cover 12420 is shown having threads 12419 extending in the horizontal direction, it should be understood that other configurations are also contemplated.

Referring to FIG. 114, the third cover 12440 can have a middle portion 12550 for attaching to a proximal end of the device 12000. In the illustrated implementation, the cover 12440 has openings 12570 for attaching to a collar of the proximal or attachment portion 12050 of the device 12000 (see FIGS. 109, 110, and 112) and the uncovered collar of the attachment portion 11050 in FIG. 103). The cover 12440 can also have coaptation portions 12590 that extend from the middle portion 12550 and are configured to cover the coaptation element 12100 of the device 12000 (see FIGS. 109, 110, and 112) and the uncovered coaptation element 11100 in FIG. 103). In the illustrated implementation, the coaptation portions have holes 12610 along their edges that allow the coaptation portions 12590 to be jointed together after being folded around the coaptation element 12100, such as, for example, by stitches or any other suitable connector.

The cover 12440 can also have end portions 12630 that extend from the coaptation portions 12590 and are configured to cover a portion of the movable arm of the clasps 12300 (see FIGS. 109, 110, and 116). The end portions 12630 can have holes 12650 along their edges that allow the end portions to be secured to the clasps 12300. The cover 12440 can include one or more cutout portions 12670 that allows for the cover 12440 to be wrapped around the proximal portion 12050, the coaptation element 12100, and the clasps 12300 in a smooth manner.

The third cover 12440 can have threads 12439 extending at an angle α (e.g., by laser cutting the cover 12440). The angle α can be between about 30 degrees and about 60 degrees, such as about 45 degrees. The angled direction of the threads 12419 can allow for connectors to be easily attached to the cover 12440 (e.g., via holes 12610, 12650), as well as allow for stretching of the cover in various directions. While the third cover 12440 is shown having threads 12439 extending at the angle α, it should be understood that other configurations are also contemplated.

Referring to FIG. 115, the first cover 12400 can have a middle portion 12690 for attaching to a distal end 12070 of the device 12000. In the illustrated implementation, the middle portion 12690 has an opening 12710 for receiving and attaching to a cap 12140 of the device 12000. The cover 12400 also includes paddle frame portions 12730 that extend from the middle portion 12690 and are configured to cover the paddle frames 12240 of each of the anchors 12080. In the illustrated implementation, the paddle frames 12240 take the form of the paddle frames 11240 shown in FIGS. 103-107, and the cover 12400 is shaped to conform to the outer frame member 12560 of the paddle frames 12240. In certain implementations, the cover 12400 can be configured to attach to both the inner and outer frame portions 12560, 12600 of the paddle frames 12240. However, it should be understood that the cover 12400 can be shaped to correspond to the shape of any suitable type of paddle frame. In implementations that include one or more sleeves 12010 (FIG. 108) attached to the paddle frames 12240, the cover 12400 can be configured to attach to the sleeves 12010. In the illustrated implementation, the first cover 12400 has holes 12732 along their edges that allow the cover 12400 to be connected to the paddle frame and/or the sleeves, such as, for example, by stitches or any other suitable connector. The first cover 12400 can also have one or more distal holes 12734 for attaching to a distal portion of the paddle frame and/or one or more proximal holes 12736 for attaching to a proximal portion of the paddle frame.

The second cover 12420 can have threads 12409 extending in a vertical direction V2 (e.g., by laser cutting the cover 12400) from a first end 12411 to a second end 12413 of the cover 12400. The horizontal direction of the threads 12409 can allow for connectors to be easily attached to the cover 12400 (e.g., via holes 12732), as well as allow for stretching of the cover in the horizontal direction H2. While the second cover 12420 is shown having threads 12409 extending in the horizontal direction, it should be understood that other configurations are also contemplated.

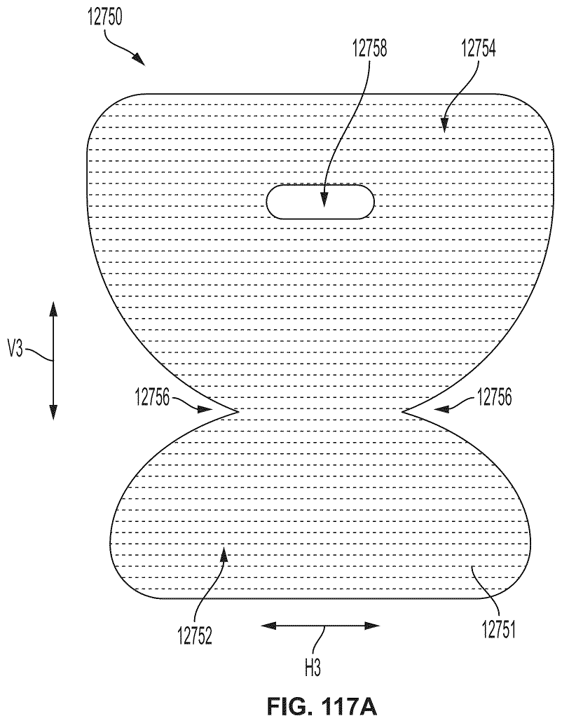

Referring to FIGS. 116 and 117, in some implementations, the cover assembly 12030 can include a clasp cover 12750 that is configured to attach to the clasp 12300 proximate the barbs 12360. The clasp cover 12750 can be configured to promote tissue ingrowth and provide a shield or buffer over the clasps 12300 as the device 12000 moves through a delivery device 12020. The placement of the clasp cover 12750 proximate or over the barbs 12360 is advantageous because the barbs 12360 engage valve tissue, and the clasp cover 12750 being configured to promote tissue ingrowth can cause the clasp cover 12750 to connect to the valve tissue. Clasp actuation lines 12770 can extend from the delivery device 12020 and attach to the clasps 12300 such that a user can move the clasps 12300 between open and closed positions. In the illustrated implementation, the connection between the clasp 12300 and the clasp actuation lines 12770 can be covered by the clasp cover 12750, which can help secure the actuation lines 12770 to the clasp 12300. For example, the clasp 12300 can have one or more holes (e.g., holes 235 shown in FIG. 26-28 of the present application) for receiving one or more clasp actuation lines 12770, and the one or more holes can be covered by the clasp cover 12750.

Referring to FIG. 117A, in some implementations, the clasp cover 12750 has a first portion 12752 for covering the barbs 12360 (FIGS. 116-117) and a second portion 12754 for folding over the free end of the movable arm of the clasp 12300 and covering the other side of the clasp 12300. The clasp cover 12750 can have one or more cutout portions 12756 that allow the cover 12750 to be wrapped around the clasps 12300 in a smooth manner. In certain implementations, the second portion 12754 includes an opening 12758 for aligning with one or more holes of the clasp 12300 such that the clasp 12300 can receive the clasp actuation lines 12770. The clasp cover 12750 can have threads 12751 extending in a horizontal direction H3 (e.g., by laser cutting the cover 12750). The horizontal direction of the threads 12751 can allow for connectors to be easily attached to the cover 12750, as well as allow for stretching of the cover in the vertical direction V3. While the clasp cover 12750 is shown having threads 12751 extending in the horizontal direction, it should be understood that other configurations are also contemplated.

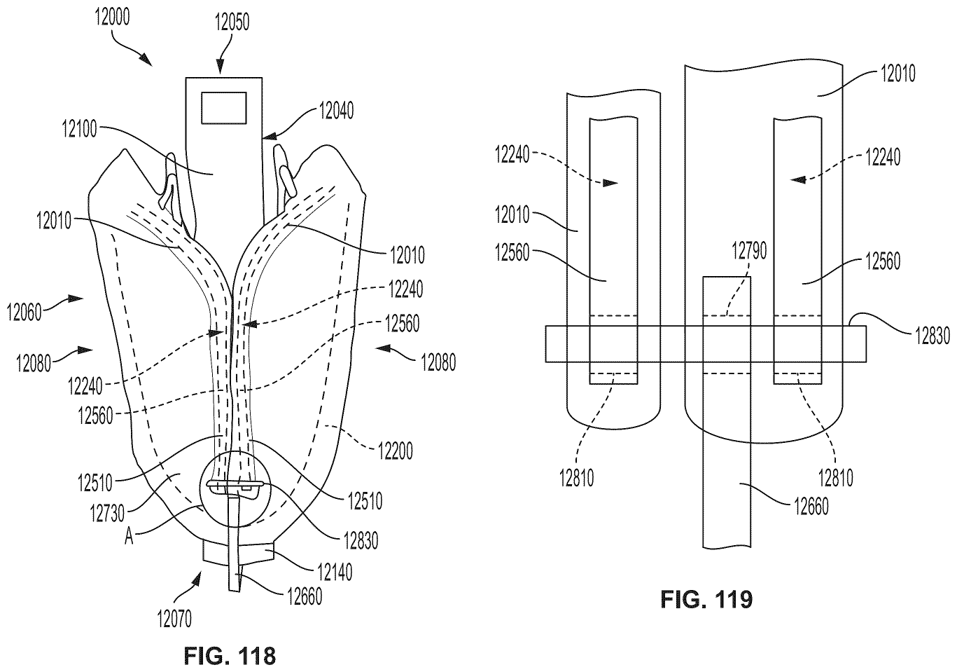

Referring to FIGS. 118 and 119, in some implementations, the device 12000 has a connector 12660 that attach the paddle frames 12240 to an actuation element of the delivery device such that the user can move the actuation element to move the inner end of the connector 12660 relative to the cap 12140 and, consequently, move the paddle frames 12240 between narrowed and expanded positions. The connectors 12660 can take the form of the connectors 11660 shown in FIGS. 103-107, or any other form described in the present application or references incorporated herein.

In the illustrated implementation, the connectors 12660 are attached to outer frame portions 12560 of the paddle frame 12240 (see the uncovered outer paddle frame portions 11560 in FIG. 103). However, other configurations are contemplated. A connection element 12830 (e.g., one or more sutures, one or more mechanical fasteners, etc.) can extend through an opening 12790 of the connectors 12660 and openings 12810 of the paddle frames to secure the paddle frames 12240 of each anchor 12080 to the connectors 12660. Referring to FIG. 119, in implementations in which each outer frame portion 12560 of each paddle frame 12240 includes a sleeve 12010, the connector 12660 and outer frame portion 12560 of one of the anchors 12080 can both be disposed in one sleeve 12010, and the outer frame portion 12560 of the other anchor 12080 can be disposed in another sleeve. In these implementations, the connection element 12830 can extend through the sleeves and the openings 12790, 12810 to secure the connector 12660 to the paddle frames 12240 of each anchor 12080.

While various inventive aspects, concepts and features of the disclosures may be described and illustrated herein as embodied in combination in some implementations, these various aspects, concepts, and features may be used in many different implementations, either individually or in various combinations and sub-combinations thereof. Unless expressly excluded herein all such combinations and sub-combinations are intended to be within the scope of the present application. Still further, while various alternative implementations as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative implementations, whether presently known or later developed. Those skilled in the art may readily adopt one or more of the inventive aspects, concepts, or features into additional implementations and uses within the scope of the present application even if such implementations are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. Further, the techniques, methods, operations, steps, etc. described or suggested herein can be performed on a living animal or on a non-living simulation, such as on a cadaver, cadaver heart, simulator (e.g., with the body parts, tissue, etc. being simulated), etc. The words used in the claims have their full ordinary meanings and are not limited in any way by the description of the implementations in the specification.

1. An implantable device comprising:
an anchor portion configured to attach to one or more leaflets of a native heart valve, the anchor portion comprising one or more anchors, wherein each anchor has a paddle frame;
one or more sleeves attached to the paddle frame; and
a cover for covering at least a portion of the paddle frame, wherein the cover is attached to the one or more sleeves.

2. The implantable device according to claim 1, wherein the one or more sleeves are lubricious to facilitate movement of the implantable device through native structures of a patient's heart.

3. The implantable device according to claim 1, wherein the one or more sleeves have a lower coefficient of friction than the paddle frame.

4. The implantable device according to claim 1, wherein the paddle frame has an inner frame portion and an outer frame portion, and wherein first and second sleeves of the one or more sleeves are attached to the inner frame portion and third and fourth sleeves of the one or more sleeves are attached to the outer frame portion.

5. The implantable device according to claim 1, wherein the cover is attached to the one or more sleeves by a plurality of stitches.

6. The implantable device according to claim 1, wherein the one or more sleeves are made of a material that promotes tissue ingrowth.

7. The implantable device according to claim 1, further comprising a coaptation element, and wherein each of the one or more anchors includes an inner paddle, an outer paddle, and a clasp.

8. The implantable device according to claim 7, wherein the cover comprises a first cover piece for covering at least a portion of the paddle frame, a second cover piece for covering at least a portion of the inner paddle, and a third cover piece for covering at least a portion of the coaptation element and the clasp.

9. The implantable device according to claim 1 wherein:
the anchor portion includes a first paddle frame, a second paddle frame, and a connector;
the one or more sleeves comprise a first sleeve and a second sleeve;
the first sleeve is disposed over at least a portion of the first paddle frame and at least a portion of the connector; and
the second sleeve is disposed over at least a portion of the second paddle frame.

10. An implantable device comprising:
a coaptation portion having a coaptation element;
an anchor portion configured to attach to one or more leaflets of a native heart valve, the anchor portion comprising a first anchor and a second anchor, wherein each of the first anchor and the second has a paddle frame, an inner paddle, an outer paddle, and a clasp;
a cover assembly that includes:
a first cover for covering at least a portion of the paddle frame of both of the first anchor and the second anchor;
a pair of second covers, wherein one second cover covers at least a portion of the inner paddle of the first anchor and the other second cover covers at least a portion of the inner paddle of the second anchor; and
a third cover for covering at least a portion of the coaptation element, at least a portion of the clasp of the first anchor, and at least a portion of the clasp of the second anchor.

11. The implantable device according to claim 10, wherein each second cover of the pair of second covers comprises a first portion disposed on the inner paddle proximate the coaptation element and a second portion that extends from the first portion and is disposed on a portion of the inner paddle that is furthest from the coaptation element.

12. The implantable device according to claim 10, wherein each second cover of the pair of second covers comprises cutout portions that assist in wrapping the second covers around the corresponding inner paddle.

13. The implantable device according to claim 10, wherein each second cover of the pair of second covers comprises a window that allows an indicator of the implantable device to be visible to a user during implantation of the implantable device.

14. The implantable device according to claim 10, wherein the third cover comprises a middle portion attached to a component at a proximal end of the implantable device, first and second coaptation portions that extend from the middle portion and cover at least a portion of the coaptation element, a first end portion that extends from the first coaptation portion and covers at least a portion of the clasp of the first anchor, and a second end portion that extends from a second coaptation portion and covers at least a portion of the clasp of the second anchor.

15. The implantable device according to claim 10, further comprising one or more sleeves attached to the paddle frame of each of the first anchor and the second anchor.

16. An implantable device comprising:
an anchor portion configured to attach to one or more leaflets of a native heart valve;
a first cover portion attached to the anchor portion;
a second cover portion attached to the anchor portion; and
wherein the second cover portion has a lower coefficient of friction than the first cover portion.

17. The implantable device according to claim 16, wherein the second cover portion covers an edge of the anchor portion.

18. The implantable device according to claim 16, wherein second cover portion comprises a hydrophilic material.

19. The implantable device according to claim 16, wherein second cover portion comprises a coating of a friction-reducing material.

20. The implantable device according to claim 19, wherein the coating is a temporary coating.

21. The implantable device according to claim 16 wherein the anchor portion comprises a plurality of paddles and the first cover portion and the second cover portion are attached to the plurality of paddles.

22. The implantable device according to claim 21, wherein the second cover portion covers an edge of each of the plurality of paddles.

23. The implantable device according to claim 16, wherein the second cover portion comprises a knitted material and wales of the knitted material are oriented longitudinally.
